SEQUENCE LISTING (34 sequences)

SEQ ID NO: 1 - protein, 750 aa, Pichia pastoris
MTGRVDQKSDQKVKELIEKIDSESTSRVFQEEPVTSILTRYEPYVAPIIFTLLSFFTRMYKIGINNHVVWDEAHFGKFGSYYLRHEFYHDVHPPLGKMLVGLSGYIAGYNGSWDFPSGQEYPDYIDYVKMRLFNATFSALCVPFAYFTMKEIGFDIKTTWLFTLMVLCETSYCTLGKFILLDSMLLLFTVTTVFTFVRFHNENSKPGNSFSRKWWKWLLLTGISIGLTCSVKMVGLFVTVLVGIYTVVDLWNKFGDQSISRKKYAAHWLARFIGLIAIPIGVFLLSFRIHFEILSNSGTGDANMSSLFQANLRGSSVGGGPRDVTTLNSKVTIKSQGLGSGLLHSHVQTYPQGSSQQQITTYSHKDANNDWVFQLTREDSRNAFKEAHYVVDGMSVRLVHSNTGRNLHTHQVAAPVSSSEWEVSCYGNETIGDPKDNWIVEIVDQYGDEDKLRLHPLTSSFRLKSATLGCYLGTSGASLPQWGFRQGEVVCYKNPFRRDKRTWWNIEDHNNPDLPNPPENFVLPRTHFLKDFVQLNLAMMATNNALVPDPDKEDNLASSAWEWPTLHVGIRLCGWGDDNVKYFLIGSPATTWTSSVGIVVFLFLLLIYLIKWQRQYVIFPSVQTPLESADTKTVALFDKSDSFNVFLMGGLYPLLGWGLHFAPFVIMSRVTYVHHYLPALYFAMIVFCYLVSLLDKKLGHPALGLLIYVALYSLVIGTFIWLSPVVFGMDGPNRNYSYLNLLPSWRVSDP

SEQ ID NO: 2 - DNA, 2250 bp, Pichia pastoris
atgacaggccgtgtcgaccagaaatctgatcagaaggtgaaggaattgatcgaaaagatcgactccgaatccacttccagagtttttcaggaagaaccagtcacttcgatcttgacacgttacgaaccctatgtcgccccaattatattcacgttgttgtcctttttcactcgtatgtacaaaattgggatcaacaaccacgtcgtttgggatgaagctcacttcggaaagtttggctcctactatctcagacacgagttctaccacgatgtccaccctccgttgggtaagatgttggtcggtctatctggctacattgccggttacaatggctcctgggatttcccctccggtcaagagtaccctgactatattgattacgttaaaatgaggttattcaatgccaccttcagtgccttatgtgtgccattcgcctatttcaccatgaaggagattggatttgatatcaagacaacttggctattcacactgatggtcttgtgtgaaacaagttattgtacgttaggaaaattcatcttgctggattcaatgctgctgctattcactgtgactacggttttcacctttgttaggttccataacgaaaacagtaaaccaggaaactcgttttctcgcaaatggtggaaatggcttctgcttactggtatttccattggtctcacttgttccgtcaaaatggtgggtttatttgtcacagtattagttggaatttacacagttgttgacttatggaataaatttggtgatcaatccatttctcgtaagaaatatgctgctcattggctagctcgtttcatcggcttgattgccatcccaattggcgtttttctattgtcattccgtatccattttgaaatattatccaattctggtaccggtgatgcaaacatgtcttcattgttccaagctaaccttcgtggatcatccgtcggaggaggccccagagatgtgaccactctcaactctaaagtgaccataaagagccaaggtttaggatctggtctgttacattcccacgttcaaacttatcctcaaggttccagccaacaacagattacaacctattctcacaaagatgccaacaatgattgggtgtttcaacttacgagagaagactctcgaaacgctttcaaggaagcccactatgtcgttgatggtatgtctgttcgtctcgttcattcaaacactggtagaaacttacacactcaccaagttgctgctcccgtctcctcatccgaatgggaagtcagttgttatggtaatgaaaccattggagacccgaaagataattggattgttgaaattgtcgaccagtatggtgatgaagataagctgagattgcacccattgacctccagtttccgtttgaaatcggcaactctgggatgctatttgggtacttcgggtgcttcactgcctcaatggggtttcagacaaggtgaagttgtttgttacaaaaatccgttccgtagagataagcgcacctggtggaacatcgaggaccataacaatcctgatctacctaatcctccagaaaattttgttcttcccaggactcattttttgaaagactttgttcaattaaatttagcaatgatggcaacaaacaacgctttggtcccagacccagataaggaagataatctagcttcttctgcctgggaatggcccacgctacacgttggtatccgtctgtgcggttggggcgatgacaacgtcaagtatttcttgattggttctcccgcaaccacctggacttcttcagttggtattgtagtattcctgttcctgctgttaatttacttgatcaaatggcaacgtcaatatgtcattttcccatccgtccagactccactagagtcagccgacaccaaaacagttgcattgtttgacaagtctgatagcttcaacgtcttccttatgggaggattatacccgcttctgggatggggtttacattttgctccgtttgtgatcatgtcgcgtgttacctacgttcaccattatcttcctgcattgtactttgccatgattgttttctgctacttggtttctctgttggataagaaactaggccacccagcattaggattactgatctatgtggctctgtattccttggtcattggaacatttatttggctcagccccgttgtgtttggtatggacggtccgaacagaaattacagttacctaaaccttctacctagttggagagtatcagaccca

SEQ ID NO: 3 - protein, 750 aa, Artificial Sequence
  Pichia pastoris Pmt2 with F664S substitution
MTGRVDQKSDQKVKELIEKIDSESTSRVFQEEPVTSILTRYEPYVAPIIFTLLSFFTRMYKIGINNHVVWDEAHFGKFGSYYLRHEFYHDVHPPLGKMLVGLSGYIAGYNGSWDFPSGQEYPDYIDYVKMRLFNATFSALCVPFAYFTMKEIGFDIKTTWLFTLMVLCETSYCTLGKFILLDSMLLLFTVTTVFTFVRFHNENSKPGNSFSRKWWKWLLLTGISIGLTCSVKMVGLFVTVLVGIYTVVDLWNKFGDQSISRKKYAAHWLARFIGLIAIPIGVFLLSFRIHFEILSNSGTGDANMSSLFQANLRGSSVGGGPRDVTTLNSKVTIKSQGLGSGLLHSHVQTYPQGSSQQQITTYSHKDANNDWVFQLTREDSRNAFKEAHYVVDGMSVRLVHSNTGRNLHTHQVAAPVSSSEWEVSCYGNETIGDPKDNWIVEIVDQYGDEDKLRLHPLTSSFRLKSATLGCYLGTSGASLPQWGFRQGEVVCYKNPFRRDKRTWWNIEDHNNPDLPNPPENFVLPRTHFLKDFVQLNLAMMATNNALVPDPDKEDNLASSAWEWPTLHVGIRLCGWGDDNVKYFLIGSPATTWTSSVGIVVFLFLLLIYLIKWQRQYVIFPSVQTPLESADTKTVALFDKSDSFNVFLMGGLYPLLGWGLHFAPSVIMSRVTYVHHYLPALYFAMIVFCYLVSLLDKKLGHPALGLLIYVALYSLVIGTFIWLSPVVFGMDGPNRNYSYLNLLPSWRVSDP

SEQ ID NO: 4 - DNA, 2250 bp, Artificial Sequence
  DNA encodes Pichia pastoris Pmt2 with F664S substitution
atgacaggccgtgtcgaccagaaatctgatcagaaggtgaaggaattgatcgaaaagatcgactccgaatccacttccagagtttttcaggaagaaccagtcacttcgatcttgacacgttacgaaccctatgtcgccccaattatattcacgttgttgtcctttttcactcgtatgtacaaaattgggatcaacaaccacgtcgtttgggatgaagctcacttcggaaagtttggctcctactatctcagacacgagttctaccacgatgtccaccctccgttgggtaagatgttggtcggtctatctggctacattgccggttacaatggctcctgggatttcccctccggtcaagagtaccctgactatattgattacgttaaaatgaggttattcaatgccaccttcagtgccttatgtgtgccattcgcctatttcaccatgaaggagattggatttgatatcaagacaacttggctattcacactgatggtcttgtgtgaaacaagttattgtacgttaggaaaattcatcttgctggattcaatgctgctgctattcactgtgactacggttttcacctttgttaggttccataacgaaaacagtaaaccaggaaactcgttttctcgcaaatggtggaaatggcttctgcttactggtatttccattggtctcacttgttccgtcaaaatggtgggtttatttgtcacagtattagttggaatttacacagttgttgacttatggaataaatttggtgatcaatccatttctcgtaagaaatatgctgctcattggctagctcgtttcatcggcttgattgccatcccaattggcgtttttctattgtcattccgtatccattttgaaatattatccaattctggtaccggtgatgcaaacatgtcttcattgttccaagctaaccttcgtggatcatccgtcggaggaggccccagagatgtgaccactctcaactctaaagtgaccataaagagccaaggtttaggatctggtctgttacattcccacgttcaaacttatcctcaaggttccagccaacaacagattacaacctattctcacaaagatgccaacaatgattgggtgtttcaacttacgagagaagactctcgaaacgctttcaaggaagcccactatgtcgttgatggtatgtctgttcgtctcgttcattcaaacactggtagaaacttacacactcaccaagttgctgctcccgtctcctcatccgaatgggaagtcagttgttatggtaatgaaaccattggagacccgaaagataattggattgttgaaattgtcgaccagtatggtgatgaagataagctgagattgcacccattgacctccagtttccgtttgaaatcggcaactctgggatgctatttgggtacttcgggtgcttcactgcctcaatggggtttcagacaaggtgaagttgtttgttacaaaaatccgttccgtagagataagcgcacctggtggaacatcgaggaccataacaatcctgatctacctaatcctccagaaaattttgttcttcccaggactcattttttgaaagactttgttcaattaaatttagcaatgatggcaacaaacaacgctttggtcccagacccagataaggaagataatctagcttcttctgcctgggaatggcccacgctacacgttggtatccgtctgtgcggttggggcgatgacaacgtcaagtatttcttgattggttctcccgcaaccacctggacttcttcagttggtattgtagtattcctgttcctgctgttaatttacttgatcaaatggcaacgtcaatatgtcattttcccatccgtccagactccactagagtcagccgacaccaaaacagttgcattgtttgacaagtctgatagcttcaacgtcttccttatgggaggattatacccgcttctgggatggggtttacattttgctccgtctgtgatcatgtcgcgtgttacctacgttcaccattatcttcctgcattgtactttgccatgattgttttctgctacttggtttctctgttggataagaaactaggccacccagcattaggattactgatctatgtggctctgtattccttggtcattggaacatttatttggctcagccccgttgtgtttggtatggacggtccgaacagaaattacagttacctaaaccttctacctagttggagagtatcagaccca

SEQ ID NO: 5 - protein, 759 aa, Saccharomyces cerevisiea
MSSSSSTGYSKNNAAHIKQENTLRQRESSSISVSEELSSADERDAEDFSKEKPAAQSSLLRLESVVMPVIFTALALFTRMYKIGINNHVVWDEAHFGKFGSYYLRHEFYHDVHPPLGKMLVGLSGYLAGYNGSWDFPSGEIYPDYLDYVKMRLFNASFSALCVPLAYFTAKAIGFSLPTVWLMTVLVLFENSYSTLGRFILLDSMLLFFTVASFFSFVMFHNQRSKPFSRKWWKWLLITGISLGCTISVKMVGLFIITMVGIYTVIDLWTFLADKSMSWKTYINHWLARIFGLIIVPFCIFLLCFKIHFDLLSHSGTGDANMPSLFQARLVGSDVGQGPRDIALGSSVVSIKNQALGGSLLHSHIQTYPDGSNQQQVTCYGYKDANNEWFFNRERGLPSWSENETDIEYLKPGTSYRLVHKSTGRNLHTHPVAAPVSKTQWEVSGYGDNVVGDNKDNWVIEIMDQRGDEDPEKLHTLTTSFRIKNLEMGCYLAQTGNSLPEWGFRQQEVVCMKNPFKRDKRTWWNIETHENERLPPRPEDFQYPKTNFLKDFIHLNLAMMATNNALVPDPDKFDYLASSAWQWPTLNVGLRLCGWGDDNPKYFLLGTPASTWASSVAVLAFMATVVILLIRWQRQYVDLRNPSNWNVFLMGGFYPLLAWGLHYMPFVIMSRVTYVHHYLPALYFALIILAYCFDAGLQKWSRSKCGRIMRFVLYAGFMALVIGCFWYFSPISFGMEGPSSNFRYLNWFSTWDIADKQEA

SEQ ID NO: 6 - DNA, 2277 bp, Saccharomyces cerevisiea
atgtcctcgtcttcgtctaccgggtacagcaaaaacaatgccgcccacattaagcaagagaatacactgagacaaagagaatcgtcttccatcagcgtcagtgaggaactttcgagcgctgatgagagagacgcggaagatttctcgaaggaaaagcccgctgcacaaagctcactgttacgcctggaatccgttgtaatgccggtgatctttactgcattggcgttgtttaccaggatgtacaaaatcggcatcaacaaccatgttgtttgggatgaggcgcactttggtaaatttggttcttattacttgagacacgaattttaccacgatgtccatcctcccctaggaaaaatgctggtcgggttgtctggttatttggcaggctacaacggttcttgggacttcccttctggggaaatttacccagactatttggattatgttaaaatgagactgttcaacgcgtcattttccgcgctctgtgtgccattggcctacttcactgccaaagctattggattttctttaccaacagtttggctgatgaccgtgttggttttgtttgaaaactcgtatagtactttgggcaggttcattcttttggactccatgctacttttcttcactgtcgcatcgttctttagttttgttatgttccacaaccagaggtccaagccgttctctagaaagtggtggaaatggctgttgatcactggtatttctttgggttgcactatttccgtcaaaatggtgggtctatttatcatcactatggtcggtatctatactgtgattgacttatggacctttttggcagataaatccatgtcatggaaaacctatattaaccactggttggcaagaatatttggtcttattatcgtccccttctgcattttcctattgtgcttcaaaatacattttgacctattatcgcattctggtacaggtgatgctaacatgccatctcttttccaagcaagattagtgggttctgacgtcggacaaggcccccgtgacattgctctaggttcctccgttgtttccatcaaaaaccaagctcttggaggatctctattgcactcacatatacaaacttatccagatgggtccaaccaacaacaagtaacctgttatggttacaaagatgctaacaacgaatggtttttcaacagagaaagaggcttaccatcatggtcagaaaacgaaactgacatcgagtatttgaagccaggtacctcctatagattggtacacaaaagcacgggcagaaacttgcacacccacccagttgctgcaccagtgtcaaagacacaatgggaggtttctggttacggtgacaatgttgttggtgacaacaaagacaattgggttattgagatcatggaccaaagaggagatgaagaccctgagaagttgcacacattgaccacctctttccgtatcaagaacttggagatgggctgttacttggctcaaaccggtaacagtttgcccgaatggggtttcagacaacaagaggttgtctgcatgaaaaacccattcaagagggacaagaggacctggtggaacatcgagacccacgaaaatgaaaggttgccaccaagacccgaagattttcaatacccaaagaccaacttcttaaaagacttcattcatttaaatctagccatgatggccactaataacgctttggtgccagatccagacaaatttgattacttagcttcctcagcatggcaatggccaactttgaatgtgggtttgagactatgtggctggggtgatgataatccaaaatacttcctattgggtaccccagcttccacgtgggcttctagtgttgccgtcctcgcattcatggccacggtcgttatcttactgatcagatggcaaagacaatatgtggacctaagaaatccatctaactggaacgttttcttaatgggcgggttctacccactactagcttggggcctacactacatgccattcgttatcatgtctagagtcacctacgttcatcattacttgcctgccttgtattttgcactgatcattttggcgtactgtttcgacgccggtttgcaaaaatggtccagatctaagtgcggccgtatcatgcggttcgtcctatacgccggattcatggcacttgtaattggttgcttctggtacttctccccaatatcatttggtatggagggaccaagtagtaacttccgctacttaaactggttttccacttgggacattgccgacaagcaagaagca

SEQ ID NO: 7 - protein, 759 aa, Artificial Sequence
  Saccharomyces cerevisiea Pmt2 with F666S substitution
MSSSSSTGYSKNNAAHIKQENTLRQRESSSISVSEELSSADERDAEDFSKEKPAAQSSLLRLESVVMPVIFTALALFTRMYKIGINNHVVWDEAHFGKFGSYYLRHEFYHDVHPPLGKMLVGLSGYLAGYNGSWDFPSGEIYPDYLDYVKMRLFNASFSALCVPLAYFTAKAIGFSLPTVWLMTVLVLFENSYSTLGRFILLDSMLLFFTVASFFSFVMFHNQRSKPFSRKWWKWLLITGISLGCTISVKMVGLFIITMVGIYTVIDLWTFLADKSMSWKTYINHWLARIFGLIIVPFCIFLLCFKIHFDLLSHSGTGDANMPSLFQARLVGSDVGQGPRDIALGSSVVSIKNQALGGSLLHSHIQTYPDGSNQQQVTCYGYKDANNEWFFNRERGLPSWSENETDIEYLKPGTSYRLVHKSTGRNLHTHPVAAPVSKTQWEVSGYGDNVVGDNKDNWVIEIMDQRGDEDPEKLHTLTTSFRIKNLEMGCYLAQTGNSLPEWGFRQQEVVCMKNPFKRDKRTWWNIETHENERLPPRPEDFQYPKTNFLKDFIHLNLAMMATNNALVPDPDKFDYLASSAWQWPTLNVGLRLCGWGDDNPKYFLLGTPASTWASSVAVLAFMATVVILLIRWQRQYVDLRNPSNWNVFLMGGFYPLLAWGLHYMPSVIMSRVTYVHHYLPALYFALIILAYCFDAGLQKWSRSKCGRIMRFVLYAGFMALVIGCFWYFSPISFGMEGPSSNFRYLNWFSTWDIADKQEA

SEQ ID NO: 8 - DNA, 2277 bp, Artificial Sequence
  DNA encodes Saccharomyces cerevisiea Pmt2 with F666S substitution
atgtcctcgtcttcgtctaccgggtacagcaaaaacaatgccgcccacattaagcaagagaatacactgagacaaagagaatcgtcttccatcagcgtcagtgaggaactttcgagcgctgatgagagagacgcggaagatttctcgaaggaaaagcccgctgcacaaagctcactgttacgcctggaatccgttgtaatgccggtgatctttactgcattggcgttgtttaccaggatgtacaaaatcggcatcaacaaccatgttgtttgggatgaggcgcactttggtaaatttggttcttattacttgagacacgaattttaccacgatgtccatcctcccctaggaaaaatgctggtcgggttgtctggttatttggcaggctacaacggttcttgggacttcccttctggggaaatttacccagactatttggattatgttaaaatgagactgttcaacgcgtcattttccgcgctctgtgtgccattggcctacttcactgccaaagctattggattttctttaccaacagtttggctgatgaccgtgttggttttgtttgaaaactcgtatagtactttgggcaggttcattcttttggactccatgctacttttcttcactgtcgcatcgttctttagttttgttatgttccacaaccagaggtccaagccgttctctagaaagtggtggaaatggctgttgatcactggtatttctttgggttgcactatttccgtcaaaatggtgggtctatttatcatcactatggtcggtatctatactgtgattgacttatggacctttttggcagataaatccatgtcatggaaaacctatattaaccactggttggcaagaatatttggtcttattatcgtccccttctgcattttcctattgtgcttcaaaatacattttgacctattatcgcattctggtacaggtgatgctaacatgccatctcttttccaagcaagattagtgggttctgacgtcggacaaggcccccgtgacattgctctaggttcctccgttgtttccatcaaaaaccaagctcttggaggatctctattgcactcacatatacaaacttatccagatgggtccaaccaacaacaagtaacctgttatggttacaaagatgctaacaacgaatggtttttcaacagagaaagaggcttaccatcatggtcagaaaacgaaactgacatcgagtatttgaagccaggtacctcctatagattggtacacaaaagcacgggcagaaacttgcacacccacccagttgctgcaccagtgtcaaagacacaatgggaggtttctggttacggtgacaatgttgttggtgacaacaaagacaattgggttattgagatcatggaccaaagaggagatgaagaccctgagaagttgcacacattgaccacctctttccgtatcaagaacttggagatgggctgttacttggctcaaaccggtaacagtttgcccgaatggggtttcagacaacaagaggttgtctgcatgaaaaacccattcaagagggacaagaggacctggtggaacatcgagacccacgaaaatgaaaggttgccaccaagacccgaagattttcaatacccaaagaccaacttcttaaaagacttcattcatttaaatctagccatgatggccactaataacgctttggtgccagatccagacaaatttgattacttagcttcctcagcatggcaatggccaactttgaatgtgggtttgagactatgtggctggggtgatgataatccaaaatacttcctattgggtaccccagcttccacgtgggcttctagtgttgccgtcctcgcattcatggccacggtcgttatcttactgatcagatggcaaagacaatatgtggacctaagaaatccatctaactggaacgttttcttaatgggcgggttctacccactactagcttggggcctacactacatgccatccgttatcatgtctagagtcacctacgttcatcattacttgcctgccttgtattttgcactgatcattttggcgtactgtttcgacgccggtttgcaaaaatggtccagatctaagtgcggccgtatcatgcggttcgtcctatacgccggattcatggcacttgtaattggttgcttctggtacttctccccaatatcatttggtatggagggaccaagtagtaacttccgctacttaaactggttttccacttgggacattgccgacaagcaagaagca

SEQ ID NO: 9 - protein, 21 aa, Artificial Sequence
  Pmt2 conserved region
PFVIMSRVTYVHHYLPALYFA

SEQ ID NO: 10 - protein, 1350 aa, Artificial Sequence
  DNA encodes Anti-RSV Heavy chain (VH + IgG1 constant region)
CAGGTTACATTGAGAGAATCCGGTCCAGCTTTGGTTAAGCCAACTCAGACTTTGACTTTGACTTGTACTTTCTCCGGTTTCTCCTTGTCTACTTCCGGAATGTCTGTTGGATGGATCAGACAACCACCTGGAAAGGCTTTGGAATGGCTTGCTGACATTTGGTGGGATGACAAGAAGGACTACAACCCATCCTTGAAGTCCAGATTGACTATCTCCAAGGACACTTCCAAGAATCAAGTTGTTTTGAAGGTTACAAACATGGACCCAGCTGACACTGCTACTTACTACTGTGCTAGATCCATGATCACTAACTGGTACTTCGATGTTTGGGGTGCTGGTACTACTGTTACTGTCTCGAGTGCTTCTACTAAGGGACCATCCGTTTTTCCATTGGCTCCATCCTCTAAGTCTACTTCCGGTGGAACCGCTGCTTTGGGATGTTTGGTTAAAGACTACTTCCCAGAGCCAGTTACTGTTTCTTGGAACTCCGGTGCTTTGACTTCTGGTGTTCACACTTTCCCAGCTGTTTTGCAATCTTCCGGTTTGTACTCTTTGTCCTCCGTTGTTACTGTTCCATCCTCTTCCTTGGGTACTCAGACTTACATCTGTAACGTTAACCACAAGCCATCCAACACTAAGGTTGACAAGAGAGTTGAGCCAAAGTCCTGTGACAAGACACATACTTGTCCACCATGTCCAGCTCCAGAATTGTTGGGTGGTCCATCCGTTTTCTTGTTCCCACCAAAGCCAAAGGACACTTTGATGATCTCCAGAACTCCAGAGGTTACATGTGTTGTTGTTGACGTTTCTCACGAGGACCCAGAGGTTAAGTTCAACTGGTACGTTGACGGTGTTGAAGTTCACAACGCTAAGACTAAGCCAAGAGAAGAGCAGTACAACTCCACTTACAGAGTTGTTTCCGTTTTGACTGTTTTGCACCAGGACTGGTTGAACGGTAAAGAATACAAGTGTAAGGTTTCCAACAAGGCTTTGCCAGCTCCAATCGAAAAGACTATCTCCAAGGCTAAGGGTCAACCAAGAGAGCCACAGGTTTACACTTTGCCACCATCCAGAGAAGAGATGACTAAGAACCAGGTTTCCTTGACTTGTTTGGTTAAAGGATTCTACCCATCCGACATTGCTGTTGAGTGGGAATCTAACGGTCAACCAGAGAACAACTACAAGACTACTCCACCAGTTTTGGATTCTGATGGTTCCTTCTTCTTGTACTCCAAGTTGACTGTTGACAAGTCCAGATGGCAACAGGGTAACGTTTTCTCCTGTTCCGTTATGCATGAGGCTTTGCACAACCACTACACTCAAAAGTCCTTGTCTTTGTCCCCTGGTTAA

SEQ ID NO: 11 - DNA, 57 bp, Artificial Sequence
  DNA encodes Saccharomyces cerevisiae mating factor pre-signal peptide
atgagattcccatccatcttcactgctgttttgttcgctgcttcttctgctttggct

SEQ ID NO: 12 - DNA, 934 bp, Artificial Sequence
  Pichia pastoris AOX1 promoter
aacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 13 - DNA, 293 bp, Artificial Sequence
  Saccahromyces cerevisiea transcription termination sequence
acaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgccggctcttaag

SEQ ID NO: 14 - DNA, 699 bp, Artificial Sequence
  DNA encodes Anti-RSV light chain (VL + Kappa constant region
atgagattcccatccatcttcactgctgttttgttcgctgcttcttctgctttggctgacattcagatgacacagtccccatctactttgtctgcttccgttggtgacagagttactatcacttgtaagtgtcagttgtccgttggttacatgcactggtatcagcaaaagccaggaaaggctccaaagttgttgatctacgacacttccaagttggcttccggtgttccatctagattctctggttccggttctggtactgagttcactttgactatctcttccttgcaaccagatgacttcgctacttactactgtttccagggttctggttacccattcactttcggtggtggtactaagttggagatcaagagaactgttgctgctccatccgttttcattttcccaccatccgacgaacaattgaagtccggtaccgcttccgttgtttgtttgttgaacaacttctacccacgtgaggctaaggttcagtggaaggttgacaacgctttgcaatccggtaactcccaagaatccgttactgagcaggattctaaggattccacttactcattgtcctccactttgactttgtccaaggctgattacgagaagcacaaggtttacgcttgcgaggttacacatcagggtttgtcctccccagttactaagtccttcaacagaggagagtgttaa

SEQ ID NO: 15 - DNA, 375 bp, Artificial Sequence
  DNA encodes Sh ble ORF (Zeocin resistance marker)
atggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 16 - DNA, 260 bp, Artificial Sequence
  Pichia pastoris AOX1 transcription termination sequence
tcaagaggatgtcagaatgccatttgcctgagagatgcaggcttcattttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagacgttcgtttgtgca

SEQ ID NO: 17 - DNA, 427 bp, Artificial Sequence
  Saccharomyces cerevisea TEF1 promoter
gatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaa

SEQ ID NO: 18 - DNA, 1793 bp, Pichia pastoris
ggtttctcaattactatatactactaaccatttacctgtagcgtatttcttttccctcttcgcgaaagctcaagggcatcttcttgactcatgaaaaatatctggatttcttctgacagatcatcacccttgagcccaactctctagcctatgagtgtaagtgatagtcatcttgcaacagattattttggaacgcaactaacaaagcagatacacccttcagcagaatcctttctggatattgtgaagaatgatcgccaaagtcacagtcctgagacagttcctaatctttaccccatttacaagttcatccaatcagacttcttaacgcctcatctggcttatatcaagcttaccaacagttcagaaactcccagtccaagtttcttgcttgaaagtgcgaagaatggtgacaccgttgacaggtacacctttatgggacattcccccagaaaaataatcaagactgggcctttagagggtgctgaagttgaccccttggtgcttctggaaaaagaactgaagggcaccagacaagcgcaacttcctggtattcctcgtctaagtggtggtgccataggatacatctcgtacgattgtattaagtactttgaaccaaaaactgaaagaaaactgaaagatgttttgcaacttccggaagcagctttgatgttgttcgacacgatcgtggcttttgacaatgtttatcaaagattccaggtaattggaaacgtttctctatccgttgatgactcggacgaagctattcttgagaaatattataagacaagagaagaagtggaaaagatcagtaaagtggtatttgacaataaaactgttccctactatgaacagaaagatattattcaaggccaaacgttcacctctaatattggtcaggaagggtatgaaaaccatgttcgcaagctgaaagaacatattctgaaaggagacatcttccaagctgttccctctcaaagggtagccaggccgacctcattgcaccctttcaacatctatcgtcatttgagaactgtcaatccttctccatacatgttctatattgactatctagacttccaagttgttggtgcttcacctgaattactagttaaatccgacaacaacaacaaaatcatcacacatcctattgctggaactcttcccagaggtaaaactatcgaagaggacgacaattatgctaagcaattgaagtcgtctttgaaagacagggccgagcacgtcatgctggtagatttggccagaaatgatattaaccgtgtgtgtgagcccaccagtaccacggttgatcgtttattgactgtggagagattttctcatgtgatgcatcttgtgtcagaagtcagtggaacattgagaccaaacaagactcgcttcgatgctttcagatccattttcccagcaggaaccgtctccggtgctccgaaggtaagagcaatgcaactcataggagaattggaaggagaaaagagaggtgtttatgcgggggccgtaggacactggtcgtacgatggaaaatcgatggacacatgtattgccttaagaacaatggtcgtcaaggacggtgtcgcttaccttcaagccggaggtggaattgtctacgattctgacccctatgacgagtacatcgaaaccatgaacaaaatgagatccaacaataacaccatcttggaggctgagaaaatctggaccgataggttggccagagacgagaatcaaagtgaatccgaagaaaacgatcaatgaacggaggacgtaagtaggaatttatg

SEQ ID NO: 19 - DNA, 957 bp, Artificial Sequence
  DNA encodes Pichia pastoris URA5 marker
tctagagggacttatctgggtccagacgatgtgtatcaaaagacaaattagagtatttataaagttatgtaagcaaataggggctaatagggaaagaaaaattttggttctttatcagagctggctcgcgcgcagtgtttttcgtgctcctttgtaatagtcatttttgactactgttcagattgaaatcacattgaagatgtcactggaggggtaccaaaaaaggtttttggatgctgcagtggcttcgcaggccttgaagtttggaactttcaccttgaaaagtggaagacagtctccatacttctttaacatgggtcttttcaacaaagctccattagtgagtcagctggctgaatcttatgctcaggccatcattaacagcaacctggagatagacgttgtatttggaccagcttataaaggtattcctttggctgctattaccgtgttgaagttgtacgagctgggcggcaaaaaatacgaaaatgtcggatatgcgttcaatagaaaagaaaagaaagaccacggagaaggtggaagcatcgttggagaaagtctaaagaataaaagagtactgattatcgatgatgtgatgactgcaggtactgctatcaacgaagcatttgctataattggagctgaaggtgggagagttgaaggttgtattattgccctagatagaatggagactacaggagatgactcaaataccagtgctacccaggctgttagtcagagatatggtacccctgtcttgagtatagtgacattggaccatattgtggcccatttgggcgaaactttcacagcagacgagaaatctcaaatggaaacgtatagaaaaaagtatttgcccaaataagtatgaatctgcttcgaatgaatgaattaatccaattatcttctcaccattattttcttctgtttcggagctttgggcacggcggcggatcc

SEQ ID NO: 20 - DNA, 709 bp, Artificial Sequence
  DNA encodes part of the Ec lacZ gene that was used to construct the PpURA5 blaster (recyclable auxotrophic marker)
cctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccag

SEQ ID NO: 21 - DNA, 1478 bp, Artificial Sequence
  DNA encodes 3′ of Pichia pastoris Pmt2p ORF and 3′ termination sequences
ctagtatttaaatgtgtatatatcgtcagtacctaaatttatgatagggtaaaaccgacatcttctatctacaattaatgcgcgactcacgctttctcttatctgcttggtcttgtttgacttcatcgttagcgttcgcttctctcttcaggtaaaaccttaccctccatgtcgacctatagagaactggagcgcgaggagggcgacttctctgataagaagcttctcacaggctggttctccttaaccgtgggacaactacaatatgctgcattcatggccgtaggaatcgcgttgctttggccttggaattgctttctatcagcttcagacttctttggagagcggttgcaagaacacaagtggctctctgctaactattcatcgtccatgatgaccatttcaacgttgacctcaacgttatgcaacgtgtttttgtcccaaaagcaaagtggggtagattattcaaagagactcgtcatgggccaaacaatcaccatagtcgtatttgcctttatgggtctgctgtgcgtatggaatacggggctagatccaataatattttttgtgttggtcatgattaatgttgcactgagttcagtagctgtgtcgttatcacaggtcggtgctatggcgatcgtgaatgtgttgggaccaatatatgctaatgctgtggttgtggggaatgctgttgctggtgtgctaccgtccatcgctctgattattagcactgccctgtcgggaactcatgtggctggaaagttgcaacctaaaagagattatgcagttatggcatactttttgactgcctgtgtcgttagtggtattgcattagttctttttgggttggcagagtcacatggcccaatcgacgttgttgctgcacctgttcatacttcttctactgctgatgaggctattgaagaactaggtatccctttagaagaggaggagtatgttcccttttcaactctgtgggccaaacttcgttttgttgcactcacaatatttacagtctttggggtttctttggtcttcccagtctttgcatcgagtattgtttccgccaacggaatcaacagtcgcatatttgtccccttggcattcctactctggaaccttggtgatttagcaggtcgattactatgtgcttatcccagatttgtcacgcgatctcccataaaattatttattttctccttagcaagatttctttacatcccgctatttgcaatttgcaatatccgagacaagggaggcctcatacagtcagatgttctatacctcctattccagctttcctttggaatatccaatggattgatttactcctcagcattcatgattgtaggagacatagcttctggagaaaatgaacagaaagctgcttcaggttttactgctgtatttttgagtcttggtctggcatgtgggtcattaggaagctacctggttgtagcatttattctttagggccc

SEQ ID NO: 22 - protein, 65 aa, Artificial Sequence
  Pichia pastoris Pmt1 highly conserved region
DPTVFNFNVQMLHYILGWVLHYLPSFLMARQLFLHHYLPSLYFGILALGHVFEIIHSYVFKNKQV

SEQ ID NO: 23 - protein, 69 aa, Artificial Sequence
  Pichia pastoris Pmt2 highly conserved region
LFDKSDSFNVFLMGGLYPLLGWGLHFAPFVIMSRVTYVHHYLPALYFAMIVFCYLVSLLDKKLGHPALG

SEQ ID NO: 24 - protein, 65 aa, Artificial Sequence
  Pichia pastoris Pmt4 highly conserved region
NSARSRLYNNLGFFFVGWCCHYLPFFLMSRQKFLHHYLPAHLIAAMFTAGFLEFIFTDNRTEEFK

SEQ ID NO: 25 - protein, 65 aa, Artificial Sequence
  Pichia pastoris Pmt6 highly conserved region
SRQYWELVIKGFVPFFGWALHFAPFIVMQRVTYVHHYVPALYFAMFLLGFTVDYLTAKRNCYIKT

SEQ ID NO: 26 - protein, 65 aa, Artificial Sequence
  Saccharomyces cerevisea Pmt1 highly conserved region
DSKVVNFHVQVIHYLLGFAVHYAPSFLMQRQMFLHHYLPAYYFGILALGHALDIIVSYVFRSKRQ

SEQ ID NO: 27 - protein, 67 aa, Artificial Sequence
  Saccharomyces cerevisea Pmt2 highly conserved region
NPSNWNVFLMGGFYPLLAWGLHYMPFVIMSRVTYVHHYLPALYFALIILAYCFDAGLQKWSRSKCGR

SEQ ID NO: 28 - protein, 65 aa, Artificial Sequence
  Saccharomyces cerevisea Pmt4 highly conserved region
KMTREKLYGPLMFFFVSWCCHYFPFFLMARQKFLHHYLPAHLIACLFSGALWEVIFSDCKSLDLE

SEQ ID NO: 29 - protein, 65 aa, Artificial Sequence
  Saccharomyces cerevisea Pmt6 highly conserved region
DDQIWQITIQGIFPFISWMTHYLPFAMMGRVTYVHHYVPALYFAMLVFGFVLDFTLTRVHWMVKY

SEQ ID NO: 30 - DNA, 1079 bp, Artificial Sequence
  pichia pastoris PMT4 5′ region
tgctctccgcgtgcaatagaaactagtcggccctgtacaattaaagcatactccctggttaaagtacctcctccgaacttgctcttgttgatcaaagtttctgaccttggggccagtccccagccaccagggccaaacgctttattgaggatacgacgatacttaatctctggaagataaagtagtccatctggtgtgatttcgacatcttcgttgctaattggttgacataatatgttactactttcattactgaaggagcaaatacctagtccatggaacgaatccgaccaattgattccatcgccacttgtattagagattggggtgtcgtttaactgtgaagttccaaacaaaattgataaactgctctcgttcttagcttggccactttttggagtctcaatagtagcgttttggctctcgtgaattttctgcacagagtcggatgaagaaggtgcaaatgcttctagcattgtagagtcgaccacatagaacctttttaaagagttatgaaaataactcttggtagggccaaatacaacccgatatcgtcttagcataagagctgcttctttggaatatcgtttcttgtaagtaattacgtgttggctaaacacttagaagtcagtcgcgcatgcggccaaaaacagactagggatagaagatgaactgacaaaaacatcaagaaggtgaagacattcattctatgaaaactagtttttatataaaattatggtctgcatttagagagcaatgatgtaatcaaacatcaataagtgcttgtcgcatcaatatttaataggtaatcatggagtattctagtctaccgccttaaaaaaagctcactcgatctagtgcagcttgattgtgtacttcaatagtattccaacgaccttaacatcttaacaccatgtaaatttaagatccacgtatacgatacaatttctttcaatatcaattctcgttcaagccaactgatgataaaatcaagaaagagatcgagaaaagtttctttgaacactgaaaaggagctgaaaaatagccatatttctcttggagatgaaagatggtacact

SEQ ID NO: 31 - DNA, 1028 bp, Artificial Sequence
  pichia pastoris PMT4 3′ region
taattcttcaaagccgaaagagcaattgattctgtggttaagtttctcgtcctttgtcgctttgctactaagcatcattgtttggactttcttcttttttgctcctctaacatatggtaatactgcgctttcggcggaggaggttcagcagcgacaatggttagatatgaagctccaattcgccaagtaagagtatacaatgtgtagttcaacgcaaaggaaattctaactttctgtgcaatctggtgacaatttctaaataactatcacaattggaagaagagattatcccaaatcttatcaaaaaatcgatgattgccagtgcacaattaggcttgaatttttcttgcagcaacgaagagattacttcagtgatgttcattagcctgaaatcttcactttcgtggtctatcggattaggaattagaccttgtttcatcggcaggtcgtatatgtattccacttctggttgaataaaatcttcgggtggtttgtttctgaacatatatgagatggctcccactggactgatatattgcgaaacatagtcctcattcaaccctgcctcctcgtaacattctttcaggcaagtttgcaaagtgccattaggatattccaagcctcctgccacagtattatctaacataccgggaaatgttggtttgtgtctgcttctcctaggtatccaaagttgaatactgttaggatcggcagaattttgcaaatatccattgatatgaactccataagtaacaactcccaaaatattagaaaaagccctttccaccaacatgtacatcttatggttatcgcagtaaactgcaaaaagctcatttctccaaccgctaagggtttcaaagagacgctgatctctccaacgctgagctatctttgcaaacatctgcgttcttttattttcggtatccagactaggaattatcttgacttcgtgtttttcattatttactatcacagcctgtgtttcgaactcaaattgttttgccaccttgggaattatataccctagt

SEQ ID NO: 32 - DNA, 1400 bp, Artificial Sequence
  DNA encodes Nat resistance expression cassette
gagttaggttcacatacgatttaggtgacactatagaacgcggccgccagctgaagcttcgtacgctgcaggtcgacggatccccgggttaattaaggcgcgccagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcatctcgtactcggcgtggaaccgccggctgaccgtcgaggacatcgaggtcgccccggagcaccgggggcacggggtcgggcgcgcgttgatggggctcgcgacggagttcgccggcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcggcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcgaattcatcgatgatatcagatccactagtggcctatgcggccgcggatctgccggtctccctatagtgagtcgtattcac

SEQ ID NO: 33 - DNA, 23 bp, Artificial Sequence
  DNA encompassing the region of the point mutation in Pichia pastoris PMT2 from YGLY17156
ttttgctccgtctgtgatcatgt

SEQ ID NO: 34 - DNA, 22 bp, Artificial Sequence
  wt PMT2 DNA from YGLY17157 encompassing the region corresponding to the region having the point mutation in Pichia pastoris strain YGLY17157
tttgctccgtttgtgatcatgt

(19) United States
(12) Patent Application Publication
(10) Pub. No.: US 2014/0302556 A1
(43) Pub. Date: Oct. 9, 2014

(54) CONTROLLING O-GLYCOSYLATION IN LOWER EUKARYOTES

(71) Applicant: MERCK SHARP & DOHEME CORP., Rahway, NJ (US)

(72) Inventors: Bo Jiang, Norwich, VT (US); Stephanie A. Nelson, White River Jct., VT (US); Rebecca D. Argyros, Hartford, VT (US); Dongxing Zha, Etna, NH (US)

(21) Appl. No.: 14/354,144

(22) PCT Filed: Oct. 22, 2012
(86) PCT No.: PCT/US12/61264

§ 371 Date: Apr. 25, 2014

Related U.S. Application Data
(60) Provisional application No. 61/552,165, filed on Oct. 27, 2011.

(51) Int. Cl. C12N 15/81; C12P 21/00

(52) U.S. Cl. CPC C12N 15/81; C12P 21/00
USPC 435/69.1; 435/254.23; 435/254.21

(57) ABSTRACT

Lower eukaryote host cells in which expression of the endogenous protein mannosyltransferase 2 (PMT2) gene has been disrupted by introducing a nucleic acid molecule encoding a Pmt2p protein having a mutation in a conserved region of the protein. The mutation confers to the host cell resistance to PMT inhibitors, which are used to reduce the amount of O-glycosylation of recombinant proteins produced by the host cells but which also have the effect of reducing the robustness of the host cells during fermentation. When host cells that express the mutated PMT2 gene but not the endogenous Pmt2p are cultivated in the presence of a P MT inhibitor, the host cells display an increase in cellular robustness during fed-batch fermentation and express recombinant pro teins in high yield while the amounts O-glycosylation are similar to that produced under similar conditions by host cells that express only the endogenous P MT2 gene.

### BACKGROUND OF THE INVENTION

(1) Field of the Invention

The present invention relates to methods for controlling O-glycosylation in lower eukaryote host cells without compromising cell robustness and protein yields. In particular, the present invention relates to lower eukaryote host cells in which expression of the endogenous protein mannosyltransferase 2 (PMT2) gene is disrupted and which has includes a nucleic acid molecule encoding a mutant Pmt2p protein having a mutation in a conserved region of the protein. The mutated Pmt2p protein confers to host cells resistance to the effects PMT inhibitors (PMTi) have on cell robustness during fermentation without inhibiting the effect of the PMT inhibitors on reducing the amount of O-glycosylation of recombinant proteins produced by the host cell.

(2) Description of Related Art

Glycoproteins mediate many essential functions in humans and other mammals, including catalysis, signaling, cell-cell communication, and molecular recognition and association. Glycoproteins make up the majority of non-cytosolic proteins in eukaryotic organisms (Lis and Sharon, 1993, Eur. J. Biochem. 218:1-27). Many glycoproteins have been exploited for therapeutic purposes, and during the last two decades, recombinant versions of naturally-occurring glycoproteins have been a major part of the biotechnology industry. Examples of recombinant glycosylated proteins used as therapeutics include erythropoietin (EPO), therapeutic monoclonal antibodies (mAbs), tissue plasminogen activator (tPA), interferon-β (IFN-β), granulocyte-macrophage colony stimulating factor (GM-CSF), and human chorionic gonadotrophin (hCH) (Cumming et al., 1991, Glycobiology 1:115-130). Variations in glycosylation patterns of recombinantly produced glycoproteins have recently been the topic of much attention in the scientific community as recombinant proteins produced as potential prophylactics and therapeutics approach the clinic.

In general, the glycosylation structures of glycoprotein oligosaccharides will vary depending upon the host species of the cells used to produce them. Therapeutic proteins produced in non-human host cells are likely to contain non-human glycosylation which may elicit an immunogenic response in humans—e.g. hypermannosylation in yeast (Ballou, 1990, Methods Enzymol. 185:440-470); α(1,3)-fucose and β(1,2)-xylose in plants, (Cabanes-Macheteau et al., 1999. Glycobiology, 9: 365-372); N-glycolylneuraminic acid in Chinese hamster ovary cells (Noguchi et al., 1995. J. Biochem. 117: 5-62); and, Galα-1,3Gal glycosylation in mice (Borrebaeck, et al., 1993, Immun. Today, 14: 477-479). Carbohydrate chains bound to proteins in animal cells include N-glycoside bond type carbohydrate chains (also called N-glycans; or N-linked glycosylation) bound to an asparagine (Asn) residue in the protein and O-glycoside bond type carbohydrate chains (also called O-glycans; or O-linked glycosylation) bound to a serine (Ser) or threonine (Thr) residue in the protein.

Because the oligosaccharide structures of glycoproteins produced by non-human mammalian cells tend to be more closely related to those of human glycoproteins, most commercial glycoproteins are produced in mammalian cells. However, mammalian cells have several important disadvantages as host cells for protein production. Besides being costly, processes for producing proteins in mammalian cells produce heterogeneous populations of glycoforms, have low volumetric titers, and require both ongoing viral containment and significant time to generate stable cell lines.

It is well recognized that the particular glycoforms on a protein can profoundly affect the properties of the protein, including its pharmacokinetic, pharmacodynamic, receptor-interaction, and tissue-specific targeting properties (Graddis et al., 2002. Curr Pharm Biotechnol. 3: 285-297). For example, it has been shown that different glycosylation patterns of Igs are associated with different biological properties (Jefferis and Lund, 1997, Antibody Eng. Chem. Immunol., 65: 111-128; Wright and Morrison, 1997, Trends Biotechnol., 15: 26-32). It has further been shown that galactosylation of a glycoprotein can vary with cell culture conditions, which may render some glycoprotein compositions immunogenic depending on the specific galactose pattern on the glycoprotein (Patel et al., 1992. Biochem J. 285: 839-845). However, because it is not known which specific glycoform(s) contribute(s) to a desired biological function, the ability to enrich for specific glycoforms on glycoproteins is highly desirable. Because different glycoforms are associated with different biological properties, the ability to enrich for glycoproteins having a specific glycoform can be used to elucidate the relationship between a specific glycoform and a specific biological function of the glycoprotein. Also, the ability to enrich for glycoproteins having a specific glycoform enables the production of therapeutic glycoproteins having particular specificities. Thus, production of glycoprotein compositions that are enriched for particular glycoforms is highly desirable.

While the pathway for N-linked glycosylation has been the subject of much analysis, the process and function of O-linked glycosylation is not as well understood. However, it is known that in contrast to N-linked glycosylation, O-glycosylation is a posttranslational event, which occurs in the cis-Golgi (Varki, 1993, Glycobiol., 3: 97-130). While a consensus acceptor sequence for O-linked glycosylation like that for N-linked glycosylation does not appear to exist, a comparison of amino acid sequences around a large number of O-linked glycosylation sites of several glycoproteins show an increased frequency of proline residues at positions −1 and +3 relative to the glycosylated residues and a marked increase of serine, threonine, and alanine residues (Wilson et al., 1991, Biochem. J., 275: 529-534). Stretches of serine and threonine residues in glycoproteins, may also be potential sites for O-glycosylation.

One gene family that has a role in O-linked glycosylation are the genes encoding the Dol-P-Man:Protein (Ser/Thr) Mannosyl Transferase (Pmtp). These highly conserved genes have been identified in both higher eukaryotes such as humans, rodents, insects, and the like and lower eukaryotes such as fungi and the like. Yeast such as Saccharomyces cerevisiae and Pichia pastoris encode up to seven PMT genes encoding Pmt homologues (reviewed in Willer et al. Curr. Opin. Struct. Biol. 2003 October; 13(5): 621-30.). In yeast, O-linked glycosylation starts by the addition of the initial mannose from dolichol-phosphate mannose to a serine or threonine residue of a nascent glycoprotein in the endoplasmic reticulum by one of the seven O-mannosyl transferases genes. While there appear to be seven PMT genes encoding Pmt homologues in yeast, O-mannosylation of secreted fungal and heterologous proteins in yeast is primarily dependent on the genes encoding Pmt1 and Pmt2, which appear to function as a heterodimer. PMT1 and PMT2 and their protein products, Pmt1 and Pmt2, respectively, appear to be highly conserved among species.

Tanner et al. in U.S. Pat. No. 5,714,377 describes the PMT1 and PMT2 genes of Saccharomyces cerevisiae and a method for making recombinant proteins having reduced O-linked glycosylation that uses fungal cells in which one or more of PMT genes have been genetically modified so that recombinant proteins are produced, which have reduced O-linked glycosylation.

Callewaert et al. in U.S. Published Application No. 20110021378 and Published International Application No. WO2010135678 discloses the PMT1, PMT2, PMT3, PMT4, PMT5, and PMT6 genes of Pichia pastoris and teaches deleting or disrupting one or more of the genes.

Ng et al. in U.S. Published Patent Application No. 20020068325 discloses inhibition of O-glycosylation through the use of antisense or cosuppression or through the engineering of yeast host strains that have loss of function mutations in genes associated with O-linked glycosylation, in particular, one or more of the PMT genes.

UDP-N-acetyl-alpha-D-galactosamine:polypeptide N-acetyl galactosaminyl-transferases (GalNAc-transferases) are involved in mucin type O-linked glycosylation found in higher eukaryotes. These enzymes initiate O-glycosylation of specific serine and threonine amino acids in proteins by adding N-acetylgalactosamine to the hydroxy group of these amino acids to which mannose residues can then be added in a step-wise manner. Clausen et al. in U.S. Pat. No. 5,871,990 and U.S. Published Patent Application No. 20050026266 discloses a family of nucleic acids encoding UDP-N-acetyl-alpha-D-galactosamine:polypeptide N-acetyl galactosaminyl-transferases (GalNAc-transferases). Clausen in U.S. Published Patent Application No. 20030186850 discloses the use of GalNAc-beta-benzyl to selectively inhibit lectins of polypeptide GalNAc-transferases and not serve as substrates for other glycosyltransferases involved in O-glycan biosyntheses, thus inhibiting O-glycosylation.

Inhibitors of O-linked glycosylation have been described. For example, Orchard et al. in U.S. Pat. No. 7,105,554 describes benzylidene thiazolidinediones and their use as antimycotic agents, e.g., antifungal agents. These benzylidene thiazolidinediones are reported to inhibit the Pmt1 enzyme, preventing the formation of the O-linked mannoproteins and compromising the integrity of the fungal cell wall. The end result is cell swelling and ultimately death through rupture.

Konrad et al. in U.S. Published Patent Application No. 20020128235 disclose a method for treating or preventing diabetes mellitus by pharmacologically inhibiting O-linked protein glycosylation in a tissue or cell. The method relies on treating a diabetic individual with (Z)-1-[N-(3-Ammoniopropyl)-N-(n-propyl)amino]diazen-ium-1,2-diolate or a derivative thereof, which binds O-linked N-acetylglucosamine transferase and thereby inhibits O-linked glycosylation.

Kojima et al. in U.S. Pat. No. 5,268,364 disclose therapeutic compositions for inhibition of O-glycosylation using compounds such as benzyle-α-N-acetylgalactosamine, which inhibits extension of O-glycosylation leading to accumulation of O-α-GalNAc, to block expression of SLex or SLea by leukocytes or tumor cells and thereby inhibit adhesion of these cells to endothelial cells and platelets.

Boime et al. in U.S. Pat. No. 6,103,501 disclose variants of hormones in which O-linked glycosylation was altered by modifying the amino acid sequence at the site of glycosylation.

Currently, to control O-glycosylation in the production of recombinant proteins in fungi, chemical inhibitors that specifically inactivate Pmtp proteins, a family of enzymes responsible for transferring the initial mannose onto the Ser/Thr residues, is supplemented to the fermentation broth in order to minimize the level of O-glycan attachment to the heterologous protein. While Orchard et al., Bioorg. Med. Chem. Lett. 14(15): 975-8 (2004) discloses benzylidene thiazolidinediones derivatives that inhibit PMT activity, U.S. Published Application No. 20090170159 first disclosed methods for using these benzylidene thiazolidinediones as PMT inhibitors to produce recombinant proteins in lower eukaryotes in which both O-glycan occupancy and O-glycan chain length are reduced. This strategy has been used to successfully control O-glycosylation of recombinant proteins produced by yeast (Kuroda et al., Appl Environ. Microbiol. 74(2):446-53 (2008); Desai and Yang, Published U.S. Application No. 20110076721). However, the use of PMT chemical inhibitors also presents several disadvantages for the fermentation and downstream processes. For example, because PMT functions are essential for yeast viability, adding PMT inhibitors to the fermentation process will invariably reduce cell fitness, which can lead to increased cell lysis and reduced protein productivity. In addition, the PMT inhibitor concentration needs to be precisely controlled during the entire production phase of the fermentation process: high levels of PMT inhibitor will result in cell death, and PMT inhibitor dosing that is too low or insufficient will most likely lead to inadequate reduction in O-glycan occupancy. Since different yeast expression strains and process platforms for cultivation (96 well plates to bioreactors) will display different levels of PMT inhibitor sensitivity, the optimal PMT inhibitor dosing scheme has to be empirically determined for each host and platform process. This introduces significant challenges for fermentation scale-up and downstream detoxification/clearance processes. However, because O-glycans may interfere with heterologous protein expression, folding, stability and, more importantly, could elicit immunogenicity in patients receiving repeated dosing, to produce safe and efficacious therapeutic proteins in fungi, the host O-glycosylation process needs to be tightly controlled.

Thus, there is a need for improved methods for controlling O-glycosylation in lower eukaryotes without compromising cell robustness and protein yields.

### BRIEF SUMMARY OF THE INVENTION

The present invention provides methods for controlling O-glycosylation in lower eukaryote host cells without compromising cell robustness and protein yields. The methods uses novel lower eukaryote host cells in which expression of the endogenous protein mannosyltransferase 2 (PMT2) gene has been disrupted and which includes a nucleic acid molecule encoding a mutant Pmt2p protein having a mutation in a conserved region of the protein. The mutation confers to the host cell resistance to PMT inhibitors. Currently, PMT inhibitors are used to reduce the amount of O-glycosylation of recombinant proteins produced by the host cells. Unfortunately though, PMT inhibitors also have the effect of reducing the fitness or robustness of the host cells during fermentation which adversely affects protein yields. However, when host cells in which expression of the endogenous PMT2 gene has been disrupted and which further include a nucleic acid molecule encoding the mutant Pmt2p protein having a mutation in a conserved region of the protein are cultivated in the presence of a PMT inhibitor, the host cells display a cellular robustness during fed-batch fermentation that is increased over that of host cells that lack the mutated PMT2 gene under similar conditions and express recombinant heterologous proteins in high yield with amounts of O-glycosylation similar to that produced by host cells that express only the endogenous PMT2 gene under similar conditions.

In general, the recombinant host cells of the present invention herein have at least one phenotype selected from the group consisting of increased cell robustness when grown in the presence of a PMT inhibitor compared to a strain that expresses the endogenous PMT2 gene and not the mutant Pmt2p protein, increased protein yield compared to a strain that expresses the endogenous PMT2 gene and not the mutant Pmt2p protein, and reduced O-glycosylation compared to a strain that expresses the endogenous PMT2 gene and not the mutant Pmt2p protein.

Thus, the present invention provides a method for producing a recombinant heterologous protein in a lower eukaryote comprising expressing a nucleic acid molecule encoding the recombinant heterologous protein in a recombinant lower eukaryote host cell in which expression of the endogenous PMT2 gene is disrupted and which comprises a nucleic acid molecule encoding a mutant Pmt2p protein comprising an amino acid substitution, deletion, or insertion in a conserved region of the Pmt2p protein comprising an amino acid sequence with at least 80%, 90%, or 95% identity to the amino acid sequence comprising the SEQ ID NO:9, to produce the recombinant heterologous protein.

In a further embodiment, the lower eukaryote is Pichia pastoris and the PMT2 gene encodes a Pmt2p protein having an amino acid sequence with at least 95% identity to the amino acid sequence of SEQ ID NO:3 with the proviso that the amino acid at position 664 is a serine residue or the lower eukaryote is Saccharomyces cerevisiae and the PMT2 gene encodes a Pmt2p having an amino acid sequence with at least 95% identity to the amino acid sequence of SEQ ID NO:7 with the proviso that the amino acid at position 666 is a serine residue. In particular aspects, the lower eukaryote host cell further does not display Pmt4p activity.

In a further embodiment, provided is method for producing a recombinant heterologous protein in a lower eukaryote comprising (a) providing a recombinant lower eukaryote host cell in which expression of the endogenous PMT2 gene is disrupted and which comprises a nucleic acid molecule encoding a mutant Pmt2p protein comprising an amino acid substitution, deletion, or insertion in a conserved region of the Pmt2p protein comprising an amino acid sequence with at least 80%, 90%, or 95% identity to the amino acid sequence comprising the SEQ ID NO:9, and a second nucleic acid molecule encoding a recombinant heterologous protein; and (b) growing the host cell in a medium comprising a Pmtp inhibitor for a time sufficient to produce the recombinant heterologous protein.

In a further embodiment, the lower eukaryote is Pichia pastoris and the PMT2 gene encodes a Pmt2p protein having an amino acid sequence with at least 95% identity to the amino acid sequence of SEQ ID NO:3 with the proviso that the amino acid at position 664 is a serine residue or the lower eukaryote is Saccharomyces cerevisiae and the PMT2 gene encodes a Pmt2p having an amino acid sequence with at least 95% identity to the amino acid sequence of SEQ ID NO:7 with the proviso that the amino acid at position 666 is a serine residue. In particular aspects, the lower eukaryote host cell further does not display Pmt4p activity.

In a further embodiment, provided is a process for producing recombinant therapeutic proteins comprising (a) providing a recombinant lower eukaryote host cell in which expression of the endogenous PMT2 gene is disrupted and which comprises a nucleic acid molecule encoding a Pmt2p protein comprising an amino acid substitution, deletion, or in a conserved region of the Pmt2p protein comprising an amino acid sequence with at least 80%, 90%, or 95% identity to the amino acid sequence comprising the SEQ ID NO:9, and second a nucleic acid molecule encoding a recombinant heterologous protein; and (b) growing the host cell in a medium comprising a Pmtp inhibitor for a time sufficient to produce the recombinant heterologous protein.

In a further embodiment, the lower eukaryote is Pichia pastoris and the PMT2 gene encodes a Pmt2p protein having the amino acid sequence of SEQ ID NO:3 or the lower eukaryote is Saccharomyces cerevisiae and the PMT2 gene encodes a Pmt2p protein having the amino acid sequence of SEQ ID NO:7. In particular aspects, the lower eukaryote host cell further does not display Pmt4p activity.

A lower eukaryote host cell comprising a disruption in the expression of the endogenous protein mannosyltransferases 2 (PMT2) gene and a nucleic acid molecule encoding a mutant Pmt2p comprising at least one amino acid substitution, deletion, or insertion in the region of the Pmt2p protein comprising a conserved region having at least 80%, 90%, or 95% identity to the amino acid sequence of SEQ ID NO:9.

In a further aspect, a serine residue replaces the phenylalanine residue at position 2 of SEQ ID NO:9.

In a further aspect, the lower eukaryote is Pichia pastoris and the PMT2 gene encodes a Pmt2p protein having an amino acid sequence with at least 95% identity to the amino acid sequence of SEQ ID NO:3 with the proviso that the amino acid at position 664 is a serine residue or the lower eukaryote is Saccharomyces cerevisiae and the PMT2 gene encodes a Pmt2p having an amino acid sequence with at least 95% identity to the amino acid sequence of SEQ ID NO:7 with the proviso that the amino acid at position 666 is a serine residue.

In a further aspect, the lower eukaryote host cell does not display Pmt4p activity.

In a further aspect, the lower eukaryote host cell comprises a nucleic acid molecule stably integrated into the genome that comprises the nucleotide sequence of SEQ ID NO:3 or the nucleotide sequence of SEQ ID NO:7. In further aspects, the nucleic acid molecule is integrated into the PMT2 gene and replaces the nucleotide sequence encoding the endogenous Pmt2p.

In particular embodiments of any one of the above host cells, the recombinant heterologous protein is therapeutic protein or glycoprotein, which in particular embodiments may be for example, selected from the group consisting of erythropoietin (EPO); cytokines such as interferon α, interferon β, interferon γ, and interferon ω; and granulocyte-colony stimulating factor (GCSF); granulocyte macrophage-colony stimulating factor (GM-CSF); coagulation factors such as factor VIII, factor IX, and human protein C; antithrombin III; thrombin; soluble IgE receptor α-chain; immunoglobulins such as IgG, IgG fragments, IgG fusions, and IgM; immunoadhesions and other Fc fusion proteins such as soluble TNF receptor-Fc fusion proteins; RAGE-Fc fusion proteins; interleukins; urokinase; chymase; urea trypsin inhibitor; IGF-binding protein; epidermal growth factor; growth hormone-releasing factor; annexin V fusion protein; angiostatin; vascular endothelial growth factor-2; myeloid progenitor inhibitory factor-1; osteoprotegerin; α-1-antitrypsin; α-feto proteins; DNase II; kringle 3 of human plasminogen; glucocerebrosidase; TNF binding protein 1; follicle stimulating hormone; cytotoxic T lymphocyte associated antigen 4-Ig; transmembrane activator and calcium modulator and cyclophilin ligand; glucagon-like protein 1; insulin, and IL-2 receptor agonist.

In further embodiments of any one of the above host cells, the therapeutic glycoprotein is an antibody, examples of which, include but are not limited to, an anti-Her2 antibody, anti-RSV (respiratory syncytial virus) antibody, anti-TNFα antibody, anti-VEGF antibody, anti-CD3 receptor antibody, anti-CD41 7E3 antibody, anti-CD25 antibody, anti-CD52 antibody, anti-CD33 antibody, anti-IgE antibody, anti-CD11a antibody, anti-EGF receptor antibody, or anti-CD20 antibody.

In further embodiments of any one of the above methods, the host cell is genetically engineered to produce glycoproteins comprising one or more N-glycans shown in FIG. 5. In further aspects of any one of the above methods, the host cell is genetically engineered to produce glycoproteins comprising one or more mammalian- or human-like complex N-glycans shown selected from G0, G1, G2, A1, or A2. In further embodiments, the host cell is genetically engineered to produce glycoproteins comprising one or more mammalian- or human-like complex N-glycans that have bisected N-glycans or have multiantennary N-glycans. In other embodiments, the host cell is genetically engineered to produce glycoproteins comprising one or more mammalian- or human-like hybrid N-glycans selected from GlcNAcMan₃GlcNAc₂; GalGlcNAcMan₃GlcNAc₂; NANAGalGlcNAcMan₃GlcNAc₂; Man₅GlcNAc₂, GlcNAcMan₅GlcNAc₂, GalGlcNAcMan₅GlcNAc₂, and NANAGalGlcNAcMan₅GlcNAc₂. In further embodiments, the N-glycan structure consists of the paucimannose (G-2) structure Man₃GlcNAc₂ or the Man₅GlcNAc₂ (GS 1.3) structure.

In particular aspects of the above host cells, the host cell includes one or more nucleic acid molecules encoding one or more catalytic domains of a glycosidase, mannosidase, or glycosyltransferase activity derived from a member of the group consisting of UDP-GlcNAc transferase (GnT) I, GnT II, GnT III, GnT IV, GnT V, GnT VI, UDP-galactosyltransferase (GalT), fucosyltransferase, and sialyltransferase. In particular embodiments, the mannosidase is selected from the group consisting of C. elegans mannosidase IA, C. elegans mannosidase IB, D. melanogaster mannosidase IA, H. sapiens mannosidase IB, P. citrinum mannosidase I, mouse mannosidase IA, mouse mannosidase IB, A. nidulans mannosidase IA, A. nidulans mannosidase IB, A. nidulans mannosidase IC, mouse mannosidase II, C. elegans mannosidase II, H. sapiens mannosidase II, and mannosidase III.

In certain aspects of any one of the above host cells, at least one catalytic domain is localized by forming a fusion protein comprising the catalytic domain and a cellular targeting signal peptide. The fusion protein can be encoded by at least one genetic construct formed by the in-frame ligation of a DNA fragment encoding a cellular targeting signal peptide with a DNA fragment encoding a catalytic domain having enzymatic activity. Examples of targeting signal peptides include, but are not limited to, those to membrane-bound proteins of the ER or Golgi, retrieval signals such as HDSL or KDEL, Type II membrane proteins, Type I membrane proteins, membrane spanning nucleotide sugar transporters, mannosidases, sialyltransferases, glucosidases, mannosyltransferases, and phospho-mannosyltransferases.

In particular aspects of any one of the above host cells, the host cell further includes one or more nucleic acid molecules encoding one or more enzymes selected from the group consisting of UDP-GlcNAc transporter, UDP-galactose transporter, GDP-fucose transporter, CMP-sialic acid transporter, and nucleotide diphosphatases.

In further aspects of any one of the above host cells, the host cell includes one or more nucleic acid molecules encoding an α1,2-mannosidase activity, a UDP-GlcNAc transferase (GnT) I activity, a mannosidase II activity, and a GnT II activity.

In further still aspects of any one of the above host cells, the host cell includes one or more nucleic acid molecules encoding an α1,2-mannosidase activity, a UDP-GlcNAc transferase (GnT) I activity, a mannosidase II activity, a GnT II activity, and a UDP-galactosyltransferase (GalT) activity.

In particular aspects, any one of the above host cells further includes a nucleic acid molecule encoding a heterologous single-subunit oligosaccharyltransferase capable of functionally suppressing the lethal phenotype of a mutation of at least one essential protein of a yeast or filamentous fungus oligosaccharyltransferase (OTase) complex. In further aspects, the single-subunit oligosaccharyltransferase is capable of functionally suppressing the lethal phenotype of a mutation of at least one essential protein of an OTase complex, for example, a yeast OTase complex. In further aspects, the essential protein of the OTase complex is encoded by the Saccharomyces cerevisiae and/or Pichia pastoris STT3 locus, WBP1 locus, OST1 locus, SWP1 locus, or OST2 locus, or homologue thereof. In particular aspects, the single-subunit oligosaccharyltransferase is the Leishmania sp. STT3A protein, STT3B protein, STT3C protein, STT3D protein, or combinations thereof. In particular aspects, the single-subunit oligosaccharyltransferase is the Leishmania major STT3A protein, STT3B protein, STT3D protein, or combinations thereof. In particular aspects, the single-subunit oligosaccharyltransferase is the Leishmania major STT3D protein. In further aspects, the single-subunit oligosaccharyltransferase is the Leishmania major STT3D protein, which is capable of functionally suppressing (or rescuing or complementing) the lethal phenotype of at least one essential protein of the Saccharomyces cerevisae OTase complex. In further aspects, the endogenous host cell genes encoding the proteins comprising the endogenous oligosaccharyltransferase (OTase) complex are expressed. In further aspects of the above host cells, which express the Leishmania major STT3D protein, the host cells further include one or more nucleic acid molecules encoding a Leishmania sp. STT3A protein, STT3B protein, STT3C protein, or combinations thereof.

In further aspects of any one of the above host cells, the host cell is selected from the group consisting of Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Ogataea minuta, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, and Neurospora crassa.

In further still aspects of any one of the above host cells, the host cell is deficient in the activity of one or more enzymes selected from the group consisting of mannosyltransferases and phosphomannosyltransferases. In further still aspects, the host cell does not express an enzyme selected from the group consisting of 1,6 mannosyltransferase, 1,3 mannosyltransferase, and 1,2 mannosyltransferase.

In a particular aspect of any one of the above host cells, the host cell is Pichia pastoris or Saccharomyces cerevisiae. In a further aspect, the host cell is an och1 mutant of Pichia pastoris or Saccharomyces cerevisiae.

### DEFINITIONS

As used herein an amino acid “modification” refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

As used herein, an “N-linked glycosylation site” refers to the tri-peptide amino acid sequence NX(S/T) or AsnXaa(Ser/Thr) wherein “N” represents an asparagine (Asn) residue, “X” represents any amino acid (Xaa) except proline (Pro), “S” represents a serine (Ser) residue, and “T” represents a threonine (Thr) residue.

As used herein, the term “N-glycan” and “glycoform” are used interchangeably and refer to the oligosaccharide group per se that is attached by an asparagine-N-acetylglucosamine linkage to an attachment group comprising an N-linked glycosylation site. The N-glycan oligosaccharide group may be attached in vitro to any amino acid residue other than asparagine or in vivo to an asparagine residue comprising an N-linked glycosylation site.

The term “N-linked glycan” refers to an N-glycan in which the N-acetylglucosamine residue at the reducing end is linked in a β1 linkage to the amide nitrogen of an asparagine residue of an attachment group in the protein.

As used herein, the terms “N-linked glycosylated” and “N-glycosylated” are used interchangeably and refer to an N-glycan attached to an attachment group comprising an asparagine residue or an N-linked glycosylation site or motif.

As used herein, the term “in vivo glycosylation” or “in vivo N-glycosylation” or “in vivo N-linked glycosylation” refers to the attachment of an oligosaccharide or glycan moiety to an asparagine residue of an N-linked glycosylation site occurring in vivo, i.e., during posttranslational processing in a glycosylating cell expressing the polypeptide by way of N-linked glycosylation. The exact oligosaccharide structure depends, to a large extent, on the host cell used to produce the glycosylated protein or polypeptide.

The term “attachment group” is intended to indicate a functional group of the polypeptide, in particular of an amino acid residue thereof, capable of being covalently linked to a macromolecular substance such as an oligosaccharide or glycan, a polymer molecule, a lipophilic molecule, or an organic derivatizing agent.

For in vivo N-glycosylation, the term “attachment group” is used in an unconventional way to indicate the amino acid residues constituting an “N-linked glycosylation site” or “N-glycosylation site” comprising N—X—S/T, wherein X is any amino acid except proline. Although the asparagine (N) residue of the N-glycosylation site is where the oligosaccharide or glycan moiety is attached during glycosylation, such attachment cannot be achieved unless the other amino acid residues of the N-glycosylation site are present. While the N-linked glycosylated insulin analogue precursor will include all three amino acids comprising the “attachment group” to enable in vivo N-glycosylation, the N-linked glycosylated insulin analogue may be processed subsequently to lack X and/or S/T. Accordingly, when the conjugation is to be achieved by N-glycosylation, the term “amino acid residue comprising an attachment group for the oligosaccharide or glycan” as used in connection with alterations of the amino acid sequence of the polypeptide is to be understood as meaning that one or more amino acid residues constituting an N-glycosylation site are to be altered in such a manner that a functional N-glycosylation site is introduced into the amino acid sequence. The attachment group may be present in the insulin analogue precursor but in the heterodimer insulin analogue one or two of the amino acid residues comprising the attachment site but not the asparagine (N) residue linked to the oligosaccharide or glycan may be removed. For example, an insulin analogue precursor may comprise an attachment group consisting of NKT at positions B28, 29, and 30, respectively, but the mature heterodimer of the analogue may be a desB30 insulin analogue wherein the T at position 30 has been removed.

As used herein, “N-glycans” have a common pentasaccharide core of Man₃GlcNAc₂ (“Man” refers to mannose; “Glc” refers to glucose; and “NAc” refers to N-acetyl; GlcNAc refers to N-acetylglucosamine). Usually, N-glycan structures are presented with the non-reducing end to the left and the reducing end to the right. The reducing end of the N-glycan is the end that is attached to the Asn residue comprising the glycosylation site on the protein. N-glycans differ with respect to the number of branches (antennae) comprising peripheral sugars (e.g., GlcNAc, galactose, fucose and sialic acid) that are added to the Man₃GlcNAc₂ (“Man₃”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid (“Sia”) or derivatives (e.g., “NANA” or “NeuAc” where “Neu” refers to neuraminic acid and “Ac” refers to acetyl, or the derivative NGNA, which refers to N-glycolylneuraminic acid). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. N-glycans consisting of a Man₃GlcNAc₂ structure are called paucimannose. The various N-glycans are also referred to as “glycoforms.”

With respect to complex N-glycans, the terms “G-2”, “G-1”, “G0”, “G1”, “G2”, “A1”, and “A2” mean the following. “G-2” refers to an N-glycan structure that can be characterized as Man₃ GlcNAc₂; the term “G-1” refers to an N-glycan structure that can be characterized as GlcNAcMan₃GlcNAc₂; the term “G0” refers to an N-glycan structure that can be characterized as GlcNAc₂Man₃GlcNAc₂; the term “G1” refers to an N-glycan structure that can be characterized as GalGlcNAc₂Man₃GlcNAc₂; the term “G2” refers to an N-glycan structure that can be characterized as Gal₂GlcNAc₂Man₃GlcNAc₂; the term “A1” refers to an N-glycan structure that can be characterized as SiₐGal₂GlcNAc₂Man₃GlcNAc₂; and, the term “A2” refers to an N-glycan structure that can be characterized as Sia₂Gal₂GlcNAc₂Man₃GlcNAc₂. Unless otherwise indicated, the terms G-2″, “G-1”, “G0”, “G1”, “G2”, “A1”, and “A2” refer to N-glycan species that lack fucose attached to the GlcNAc residue at the reducing end of the N-glycan. When the term includes an “F”, the “F” indicates that the N-glycan species contain a fucose residue on the GlcNAc residue at the reducing end of the N-glycan. For example, G0F, G1F, G2F, A1F, and A2F all indicate that the N-glycan further includes a fucose residue attached to the GlcNAc residue at the reducing end of the N-glycan. Lower eukaryotes such as yeast and filamentous fungi do not normally produce N-glycans that produce fucose.

With respect to multiantennary N-glycans, the term “multiantennary N-glycan” refers to N-glycans that further comprise a GlcNAc residue on the mannose residue comprising the non-reducing end of the 1,6 arm or the 1,3 arm of the N-glycan or a GlcNAc residue on each of the mannose residues comprising the non-reducing end of the 1,6 arm and the 1,3 arm of the N-glycan. Thus, multiantennary N-glycans can be characterized by the formulas GlcNAc₍₂₋₄₎Man₃GlcNAc₂, Gal₍₁₋₄₎GlcNAc₍₂₋₄₎Man₃GlcNAc₂, or Sia₍₁₋₄₎Gal₍₁₋₄₎GlcNAc₍₂₋₄₎Man₃GlcNAc₂. The term “1-4” refers to 1, 2, 3, or 4 residues.

With respect to bisected N-glycans, the term “bisected N-glycan” refers to N-glycans in which a GlcNAc residue is linked to the mannose residue at the non-reducing end of the N-glycan. A bisected N-glycan can be characterized by the formula GlcNAc₃Man₃GlcNAc₂ wherein each mannose residue is linked at its non-reducing end to a GlcNAc residue. In contrast, when a multiantennary N-glycan is characterized as GlcNAc₃Man₃GlcNAc₂, the formula indicates that two GlcNAc residues are linked to the mannose residue at the non-reducing end of one of the two arms of the N-glycans and one GlcNAc residue is linked to the mannose residue at the non-reducing end of the other arm of the N-glycan.

Abbreviations used herein are of common usage in the art, see, e.g., abbreviations of sugars, above. Other common abbreviations include “PNGase”, or “glycanase” which all refer to glycopeptide N-glycosidase; glycopeptidase; N-oligosaccharide glycopeptidase; N-glycanase; glycopeptidase; Jack-bean glycopeptidase; PNGase A; PNGase F; glycopeptide N-glycosidase (EC 3.5.1.52, formerly EC 3.2.2.18).

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. Host cells may be yeast, fungi, mammalian cells, plant cells, insect cells, and prokaryotes and archaea that have been genetically engineered to produce glycoproteins.

When referring to “mole percent” or “mole %” of a glycan present in a preparation of a glycoprotein, the term means the molar percent of a particular glycan present in the pool of N-linked oligosaccharides released when the protein preparation is treated with PNGase and then quantified by a method that is not affected by glycoform composition, (for instance, labeling a PNGase released glycan pool with a fluorescent tag such as 2-aminobenzamide and then separating by high performance liquid chromatography or capillary electrophoresis and then quantifying glycans by fluorescence intensity). For example, 50 mole percent GlcNAc₂Man₃GlcNAc₂Gal₂NANA₂ means that 50 percent of the released glycans are GlcNAc₂Man₃GlcNAc₂Gal₂NANA₂ and the remaining 50 percent are comprised of other N-linked oligosaccharides. In embodiments, the mole percent of a particular glycan in a preparation of glycoprotein will be between 20% and 100%, preferably above 25%, 30%, 35%, 40% or 45%, more preferably above 50%, 55%, 60%, 65% or 70% and most preferably above 75%, 80% 85%, 90% or 95%.

The term “operably linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences that are necessary to affect the expression of coding sequences to which they are operably linked. Expression control sequences are sequences that control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “transfect”, “transfection”, “transfecting” and the like refer to the introduction of a heterologous nucleic acid into eukaryote cells, both higher and lower eukaryote cells. Historically, the term “transformation” has been used to describe the introduction of a nucleic acid into a prokaryote, yeast, or fungal cell; however, the term “transfection” is also used to refer to the introduction of a nucleic acid into any prokaryotic or eukaryote cell, including yeast and fungal cells. Furthermore, introduction of a heterologous nucleic acid into prokaryotic or eukaryotic cells may also occur by viral or bacterial infection or ballistic DNA transfer, and the term “transfection” is also used to refer to these methods in appropriate host cells.

The term “eukaryotic” refers to a nucleated cell or organism, and includes insect cells, plant cells, mammalian cells, animal cells and lower eukaryotic cells.

The term “lower eukaryotic cells” includes fungal cells, which include yeast and filamentous fungi. Yeast and filamentous fungi include, but are not limited to Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorphs, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., Hansenula polymorpha, any Kluyveromyces sp., Candida albicans, any Aspergillus sp., Trichoderma reesei, Chrysosporium lucknowense, any Fusarium sp., Yarrowia lipolytica, and Neurospora crassa.

As used herein, the term “consisting essentially of” will be understood to imply the inclusion of a stated integer or group of integers; while excluding modifications or other integers that would materially affect or alter the stated integer. For example, with respect to a species of N-glycans attached to an insulin or insulin analogue, the term “consisting essentially of” a stated N-glycan will be understood to include the N-glycan whether or not that N-glycan is fucosylated at the N-acetylglucosamine (GlcNAc) which is directly linked to the asparagine residue of the glycoprotein provided that for the particular N-glycan species the fucose does not materially affect the glycosylated insulin or insulin analogue compared to the glycosylated insulin or insulin analogue in which the N-glycan lacks the fucose.

As used herein, the term “predominantly” or variations such as “the predominant” or “which is predominant” will be understood to mean the glycan species that has the highest mole percent (%) of total neutral N-glycans after the insulin analogue has been treated with PNGase and released glycans analyzed by mass spectroscopy, for example, MALDI-TOF MS or HPLC. In other words, the phrase “predominantly” is defined as an individual entity, such as a specific glycoform, is present in greater mole percent than any other individual entity. For example, if a composition consists of species A at 40 mole percent, species B at 35 mole percent and species C at 25 mole percent, the composition comprises predominantly species A, and species B would be the next most predominant species. Some host cells may produce compositions comprising neutral N-glycans and charged N-glycans such as mannosylphosphate. Therefore, a composition of glycoproteins can include a plurality of charged and uncharged or neutral N-glycans. In the present invention, it is within the context of the total plurality of neutral N-glycans in the composition in which the predominant N-glycan determined. Thus, as used herein, “predominant N-glycan” means that of the total plurality of neutral N-glycans in the composition, the predominant N-glycan is of a particular structure.

As used herein, the term “essentially free of” a particular sugar residue, such as fucose, or galactose and the like, is used to indicate that the glycoprotein composition is substantially devoid of N-glycans which contain such residues. Expressed in terms of purity, essentially free means that the amount of N-glycan structures containing such sugar residues does not exceed 10%, and preferably is below 5%, more preferably below 1%, most preferably below 0.5%, wherein the percentages are by weight or by mole percent. Thus, substantially all of the N-glycan structures in an insulin analogue composition disclosed herein are free of, for example, fucose, or galactose, or both.

As used herein, a protein or glycoprotein composition “lacks” or “is lacking” a particular sugar residue, such as fucose or galactose, when no detectable amount of such sugar residue is present on the N-glycan structures at any time. For example, in preferred embodiments of the present invention, the protein or glycoprotein are produced by lower eukaryotic organisms, as defined above, including yeast (for example, Pichia sp.; Saccharomyces sp.; Kluyveromyces sp.; Aspergillus sp.), and will “lack fucose,” because the cells of these organisms do not have the enzymes needed to produce fucosylated N-glycan structures. Thus, the term “essentially free of fucose” encompasses the term “lacking fucose.” However, a composition may be “essentially free of fucose” even if the composition at one time contained fucosylated N-glycan structures or contains limited, but detectable amounts of fucosylated N-glycan structures as described above.

### BRIEF DESCRIPTION OF THE DRAWINGS

FIG. 1 illustrates the procedure used to obtain the strains showing resistance to the PMT inhibitor PMTi-4 (strains YGLY17156 and YGLY17157) produced.

FIG. 2 shows the that the IgG produced by the strains resistant to the PMT inhibitor PMTi-4 (strains YGLY17156 and YGLY17157) produced higher amounts of fully assembled IgG1 than the non-mutagenized parent strain YGLY19376.

FIG. 3 shows the thymidine (T) to cytosine (C) point mutation at position 1991 in the nucleotide sequence encoding the Pmt2p protein from strain YGLY17156 (SEQ ID NO:33) compared to the corresponding region encoding Pmt2p protein from YGLY17157 (SEQ ID NO:34).

FIG. 4 shows that point mutation effects a change in the amino acid at position 664 of the Pmt2p protein, which resides in a highly conserved region as determined by an alignment of the amino acid sequences for Pmt1p, Pmt2p, Pmt4p, and Pmt6p from Pichia pastoris (Pp) and Saccharomyces cerevisiae (Sc). PpPMT1 (SEQ ID NO:22), PpPMT2 (SEQ ID NO:23), PpPMT4 (SEQ ID NO:24), PpPMT6 (SEQ ID NO:25), ScPMT1 (SEQ ID NO:26), ScPMT2 (SEQ ID NO:27), ScPMT4 (SEQ ID NO:28), and ScPMT6 (SEQ ID NO:29).

FIG. 5 shows examples of N-glycan structures that can be attached to the asparagine residue in the motif Asn-Xaa-Ser/Thr wherein Xaa is any amino acid other than proline or attached to any amino acid in vitro. Recombinat host cells can be genetically modified to produce glycoproteins that have predominantly particular N-glycan species.

FIG. 6 shows a map of plasmid vector pGLY5931 designed to replace the genomic nucleotide sequence encoding the endogenous Pmt2p protein with a nucleotide sequence that encodes the Pmt2p-F664S mutant protein. The vector includes the URA5 gene to enable selection of Ura+ recombinants when transformed into a strain that is URA5 auxotroph.

FIG. 7 shows that transforming several PMTi-4-sensitive strains with plasmid pGLY5931 transformed the strains into PMTi-4-resistant strains.

FIG. 8 shows a map of plasmid vector pGLY4857 designed to disrupt the PMT4 locus.

### DETAILED DESCRIPTION OF THE INVENTION

The present invention provides methods for controlling O-glycosylation in lower eukaryote host cells without compromising cell robustness and protein yields. In particular, the present invention provides lower eukaryote host cells in which expression of the endogenous Dol-P-Man:Protein (Ser/Thr) Mannosyl Transferase 2 gene (or protein mannosyltransferase 2 (PMT2) gene) is disrupted and which have been transformed with a nucleic acid molecule encoding a Pmt2p protein having a mutation in a conserved region of the protein that confers to the host cell resistance to PMT inhibitors (mutated Pmt2p), which are used to reduce the amount of O-glycosylation of recombinant heterologous proteins produced by the host cells but which also have the effect of reducing the robustness of the host cells during fermentation. When host cells that have the express the mutated Pmt2p are cultivated in the presence of a PMT inhibitor, the host cells display a cellular robustness during fed-batch fermentation that is increased over that of host cells that lack the mutated PMT2 gene under similar conditions and express recombinant heterologous proteins in high yield with a level of O-glycosylation that is similar to that produced under similar conditions by host cells that have the endogenous PMT2 gene.

As used herein, disruption of endogenous PMT2 expression includes but is not limited to deleting the PMT2 gene, disrupting the coding region of the PMT2 gene, or mutating the PMT2 gene to an extent that the encoded Pmt2p is nonfunctional.

In general, in the embodiments disclosed herein, the nucleic acid molecule encoding the mutated Pmt2p protein is stably integrated into the genome of the host cell by double or single crossover homologous recombination. In particular embodiments, the nucleic acid molecule encoding the mutated Pmt2p protein is integrated into the open reading frame of the endogenous PMT2 gene encoding the endogenous Pmt2p, which results in replacement of the nucleic acid sequences encoding the endogenous Pmt2p with the nucleic acid sequences encoding the mutated Pmt2p. In that embodiment, the expression of the nucleic acid molecule encoding the mutated Pmt2p is under the control of the endogenous PMT2 gene regulatory sequences.

The present invention provides a genetic solution for O-glycosylation control in lower eukaryotes. As shown in Example 1, using a random mutagenesis approach and Pichia pastoris genetically engineered to produce glycoproteins that have predominantly human-like N-glycans as a model, a Pichia pastoris mutant strain was isolated that was highly resistant to PMT inhibitors when compared to the non-mutagenized parent strain. When tested under conditions of regular PMT inhibitor dosing supplementation during fermentation, this mutant strain displayed increased cell robustness and recombinant heterologous protein expression when compared to the non-mutagenized parent strain, and produced recombinant heterologous proteins with a reduced O-glycosylation that was comparable to that produced by the non-mutagenized parent strain grown under similar conditions. Interestingly and unexpected, even in the absence of any PMT inhibitor, this mutant strain was still capable of producing recombinant heterologous proteins with a level of O-glycan occupancy that was at least four-fold lower than that produced by the non-mutagenized parent strain.

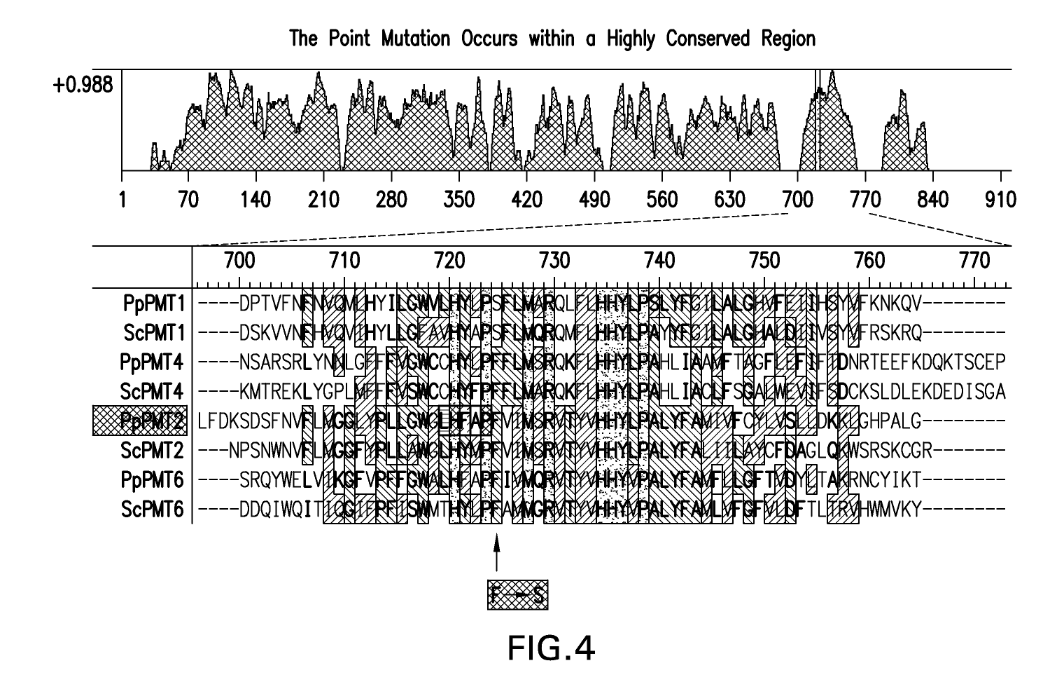

As shown in Example 1, the observed phenotype of PMT inhibitor-resistance or tolerance, increased protein expression, increased cell robustness, and O-glycosylation reduction, was found to be the result of a single point-mutation within the nucleotide sequence encoding the Pmt2p protein. The single point-mutation was a “T” to a “C” nucleotide transition at position 1991 in the open reading frame (ORF) encoding the Pmt2p protein (PMT2-T1991C point mutation), which results in an amino acid change at position 664 of the Pmt2p from phenylalanine encoded by the codon TTT to serine encoded by the codon TCT (Pmt2p-F664S mutant protein). When the wild-type PMT2 gene in Pichia pastoris was replaced with a nucleic acid molecule encoding Pmt2p-F664S mutant protein, the phenotypic effect that had been observed in the mutant strain was reproduced. The F664S mutation occurs in a highly conserved region of the Pmt2p protein as shown in FIG. 4. The highly conserved region comprises the amino acid sequence PFVIMSRVTYVHHYLPALYFA (SEQ ID NO:9). Thus, for Saccharomyces cerevisiae, replacing the endogenous PMT2 gene with a nucleic acid molecule encoding a Pmt2p protein with an F666S mutation (Pmt2p-F666S mutant protein) may confer a phenotype similar to that had been observed with Pichia pastoris. The results in the Examples suggest that a mutation anywhere within the nucleotide sequence encoding the highly conserved region may confer the observed phenotype to the host cell. Mutations that include substitution of the phenylalanine at position two of SEQ ID NO:9 with another amino acid, for example serine, or the substitution, deletion, or insertion of at least one amino acid residue any where within the highly conserved region may have broad utility for any heterologous protein-expressing yeast host strain in which the desired phenotype is to include a reduction in protein O-glycosylation; increased PMT inhibitor-resistance or -tolerance; and increased strain robustness and viability during fermentation.

Control of O-glycosylation using the host cells disclosed herein is useful for producing particular glycoproteins such as antibodies in the host cells disclosed herein in better total yield or in yield of properly assembled glycoprotein. The reduction or elimination of O-glycosylation appears to have a beneficial effect on the assembly and transport of glycoproteins such as whole antibodies as they traverse the secretory pathway and are transported to the cell surface. Thus, in cells in which O-glycosylation is controlled, the yield of properly assembled glycoproteins such as antibody fragments is increased over the yield obtained in host cells in which O-glycosylation is not controlled. Some mammalian and human proteins contain sequences which may not be O-glycosylated in the native host cell but which are O-glycosylated when the protein is expressed in a lower eukaryote such as yeast. For example, insulin is not normally considered a glycoprotein since it lacks N-linked glycosylation sites; however, when insulin is produced in yeast, a small population of the insulin synthesized appears to be O-glycosylated: methods for removal of these O-glycosylated molecules have been developed for insulin expressed in Pichia pastoris or Saccharomyces cerevisiae (See for example, International Published Application No. and WO2009104199 and U.S. Pat. No. 6,180,757, respectively). Therefore, control of O-glycosylation using the host cells herein are also useful for producing proteins and glycoproteins with little or no unwanted O-glycosylation.

Methods in the art for controlling O-glycosylation include deleting or disrupting one or more of the endogenous PMT genes (See U.S. Pat. No. 5,714,377). While deletion of either the PMT1 or PMT2 genes in a lower eukaryote host cell enables production of a recombinant heterologous protein having reduced O-linked glycosylation in the host cell, expression of the PMT1 and PMT2 genes are important for host cells growth and either deletion alone also adversely affects the ability of the host cell to grow thus making it difficult to produce a sufficient quantity of host cells or recombinant heterologous protein with a reduced amount of O-linked glycosylation. This effect is particularly evident in lower eukaryote host cells genetically engineered to lack alpha-1,6-mannosyltransferase activity encoded by the OCH1 gene and to include mammalian and human glycosylation enzymes and pathways for producing glycoproteins with a human-like glycosylation pattern or having predominantly particular N-glycan structures. Deletion of both genes appears to be lethal to the lower eukaryote host cell. Therefore, genetic elimination of the PMT1 and PMT2 genes in a lower eukaryote host cell would appear to be an undesirable means for producing recombinant heterologous proteins having reduced O-linked glycosylation.

Thus, methods were developed in which the lower eukaryote host cell is grown in the presence of one or more Pmtp inhibitors alone or in the presence of an alpha-mannosidase modified to be secreted from the host cell (See, for example, Published International Application No. WO2007061631). Examples of Pmtp inhibitors include but are not limited to a benzylidene thiazolidinediones such as those disclosed in U.S. Pat. No. 7,105,554 and U.S. Published Application No. 20110076721. Examples of benzylidene thiazolidinediones that can be used are 5-[[3,4-bis(phenylmethoxy)phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; 5-[[3-(1-Phenyl-2-hydroxy)ethoxy)-4-(2-phenylethoxy)]phenyl]methylene]-4-oxo-2-thioxo-3-thiazolidineacetic Acid; and, Example 4 compound in U.S. Published Application No. 20110076721). However, while these methods have been successful in controlling O-glycosylation, these PMT inhibitors do reduce cell viability which in turn affects recombinant protein yields.

The present invention provides a recombinant lower eukaryote host cell that expresses a mutant Pmt2p protein that has a mutation in a highly conserved region of the protein and does not express its endogenous Pmt2p protein. The recombinant lower eukaryote host cell displays at least one phenotype selected from the group consisting of increased cell robustness when grown in the presence of a PMT inhibitor compared to a strain that expresses the endogenous PMT2 gene and not the mutant Pmt2p protein, increased protein yield compared to a strain that expresses the endogenous PMT2 gene and not the mutant Pmt2p protein, and reduced O-glycosylation compared to a strain that expresses the endogenous PMT2 gene and not the mutant Pmt2p protein. In general, the recombinant host cell further includes a nucleic acid molecule encoding a recombinant heterologous protein, which in particular embodiments is a therapeutic protein.

In particular embodiments, provided is a recombinant lower eukaryote host cell comprising a disruption of expression of the endogenous PMT2 gene and a nucleic acid molecule encoding a mutant Pmt2p protein comprising at least one amino acid substitution, deletion, or insertion in the region of the Pmt2p protein comprising a conserved region having an amino acid sequence with at least 80%, 90%, or 95% identity to SEQ ID NO:9. In further embodiments, the recombinant host cell further includes a nucleic acid molecule encoding a recombinant heterologous protein, which in further embodiments is a therapeutic protein.

In particular embodiments, provided is a lower eukaryote host cell comprising a disruption of expression of the endogenous PMT2 gene and a nucleic acid molecule encoding a mutant Pmt2p protein comprising a conserved region having at least at least 80%, 90%, or 95% identity to the amino acid sequence of SEQ ID NO:9 in which the phenylalanine residue at position two of SEQ ID NO:9 is substituted with a serine residue. In further embodiments, the recombinant host cell further includes a nucleic acid molecule encoding a recombinant heterologous protein, which in further embodiments is a therapeutic protein.

In particular embodiments, provided is a recombinant lower eukaryote host cell comprising a disruption of expression of the endogenous PMT2 gene and a nucleic acid molecule encoding a mutant Pmt2p protein comprising a conserved region having the amino acid sequence of SEQ ID NO:9 in which the phenylalanine residue at position two of SEQ ID NO:9 is substituted with a serine residue. In further embodiments, the recombinant host cell further includes a nucleic acid molecule encoding a recombinant heterologous protein, which in further embodiments is a therapeutic protein.

In particular aspects of the above, the recombinant host cell is selected from the group consisting of Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Ogataea minuta, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, and Neurospora crassa. Various yeasts, such as Ogataea minuta, Kluyveromyces lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale.

In further still aspects, the recombinant host cell is deficient in the activity of one or more enzymes selected from the group consisting of mannosyltransferases and phosphomannosyltransferases. In further still aspects, the host cell does not express an enzyme selected from the group consisting of 1,6 mannosyltransferase, 1,3 mannosyltransferase, and 1,2 mannosyltransferase.

In particular aspects of any one of the above recombinant host cell, the recombinant host cell is Pichia pastoris or Saccharomyces cerevisiae. In a further aspect, the recombinant host cell is an och1 mutant of Pichia pastoris or Saccharomyces cerevisiae.

In particular embodiments, provided is a recombinant Pichia pastoris host cell comprising a disruption of expression of the endogenous PMT2 gene and a nucleic acid molecule encoding a mutant Pmt2p protein comprising a substitution of the phenylalanine residue at position 664 of the Pmt2p protein with an serine residue. In further embodiments, the recombinant host cell further include a nucleic acid molecule encoding a recombinant heterologous protein, which in further embodiments is a therapeutic protein.

In particular embodiments, provided is a recombinant Saccharomyces cerevisiae host cell comprising a disruption of expression of the endogenous PMT2 gene and a nucleic acid molecule encoding a mutant Pmt2p protein comprising a substitution of the phenylalanine residue at position 666 of the Pmt2p protein with a serine residue. In further embodiments, the recombinant host cell further includes a nucleic acid molecule encoding a recombinant heterologous protein, which in further embodiments is a therapeutic protein.

In particular embodiments, provided is a recombinant Pichia pastoris host cell comprising a disruption of expression of the endogenous PMT2 gene and a nucleic acid molecule encoding a mutant Pmt2p protein comprising at least 90%, 95%, 96%, 97%, 98% 99%, or 100% identity to the amino acid sequence of SEQ ID NO:3 with the proviso that the amino acid at position 664 is a serine residue. In further embodiments, the recombinant host cell further includes a nucleic acid molecule encoding a recombinant heterologous protein, which in further embodiments is a therapeutic protein.

In particular embodiments, provided is a recombinant Saccharomyces cerevisiae host cell comprising a disruption of expression of the endogenous PMT2 gene and a nucleic acid molecule encoding a mutant Pmt2p protein comprising at least 90%, 95%, 96%, 97%, 98% 99%, or 100% identity to the amino acid sequence of SEQ ID NO:7 with the proviso that the amino acid at position 666 is a serine residue. In further embodiments, the recombinant host cell further includes a nucleic acid molecule encoding a recombinant heterologous protein, which in further embodiments is a therapeutic protein.

In further embodiments, any one of the above host cells may further include a reduction, disruption, or deletion of the function or expression of at least one endogenous PMT gene selected from PMT1, PMT3, PMT4, and PMT6. In particular aspects, the above host cell comprises a deletion or disruption of the PMT4 gene.

In further embodiments, the host cell further includes a nucleic acid molecule encoding an α-1,2-mannosidase that targets the secretory pathway and is secreted by the host cell. In particular aspects, a chimeric α-1,2-mannosidase is provided wherein the catalytic domain of the α-1,2-mannosidase is fused to a heterologous targeting peptide that targets the chimeric α-1,2-mannosidase to the secretory pathway and the chimeric α-1,2-mannosidase is secreted from the host cell. In particular aspects, the α-1,2-mannosidase is from Trichoderma reesei, Saccharomyces sp., or Aspergillus sp. In further aspects, the targeting peptide is Saccharomyces cerevisiae alpha-mating factor pre-signal peptide. In this embodiment, the α-1,2-mannosidase or chimeric α-1,2-mannosidase, which is secreted, reduces the chain length of any O-glycans that may be present even in host cells having any combination of the above mutations and/or deletions to about one mannose residue per O-glycan.

The present invention further provides methods for producing recombinant heterologous proteins in the lower eukaryote host cell supra wherein the recombinant lower eukaryote host cell displays at least one phenotype selected from the group consisting of increased cell robustness when grown in the presence of a PMT inhibitor compared to a strain that expresses the endogenous PMT2 gene and not the mutant Pmt2p protein, increased protein yield compared to a strain that expresses the endogenous PMT2 gene and not the mutant Pmt2p protein, and reduced O-glycosylation compared to a strain that expresses the endogenous PMT2 gene and not the mutant Pmt2p protein.

In particular embodiments, provided is a method for producing a recombinant heterologous protein in a lower eukaryote host cell comprising expressing a nucleic acid molecule encoding the recombinant heterologous protein in a recombinant lower eukaryote host cell comprising a disruption in the expression of the endogenous PMT2 gene and a nucleic acid molecule encoding a mutant Pmt2p comprising at least one amino acid substitution, deletion, or insertion in the region of the Pmt2p protein comprising a conserved region having an amino acid sequence with at least 80%, 90%, or 95% identity to SEQ ID NO:9 to produce the recombinant heterologous protein.

In particular embodiments, provided is a method for producing a recombinant heterologous protein in a lower eukaryote comprising expressing a nucleic acid molecule encoding the recombinant heterologous protein in a recombinant host cell in which expression of the endogenous PMT2 gene is disrupted and which comprises a nucleic acid molecule encoding a Pmt2p protein comprising a conserved region having an amino acid sequence with at least 80%, 90%, or 95% identity to the amino acid sequence of SEQ ID NO:9 in which the phenylalanine residue at position two of SEQ ID NO:9 is substituted with a serine residue to produce the recombinant heterologous protein.

In particular embodiments, provided is a method for producing a recombinant heterologous protein in a lower eukaryote comprising expressing a nucleic acid molecule encoding the recombinant heterologous protein in a recombinant host cell in which expression of the endogenous PMT2 gene is disrupted and which comprises a nucleic acid molecule encoding a Pmt2p protein comprising a conserved region having the amino acid sequence of SEQ ID NO:9 in which the phenylalanine residue at position two of SEQ ID NO:9 is substituted with a serine residue to produce the recombinant heterologous protein.

In particular aspects of the above, the host cell is selected from the group consisting of Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Ogataea minuta, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, and Neurospora crassa. Various yeasts, such as Ogataea minuta, Kluyveromyces lactis, Pichia pastoris, Pichia methanolica, and Hansenula polymorpha are particularly suitable for cell culture because they are able to grow to high cell densities and secrete large quantities of recombinant heterologous protein. Likewise, filamentous fungi, such as Aspergillus niger, Fusarium sp, Neurospora crassa and others can be used to produce glycoproteins of the invention at an industrial scale.

In further still aspects, the host cell is deficient in the activity of one or more enzymes selected from the group consisting of mannosyltransferases and phosphomannosyltransferases. In further still aspects, the host cell does not express an enzyme selected from the group consisting of 1,6 mannosyltransferase, 1,3 mannosyltransferase, and 1,2 mannosyltransferase.

In particular, aspects of any one of the above host cells, the host cell is Pichia pastoris or Saccharomyces cerevisiae. In a further aspect, the host cell is an och1 mutant of Pichia pastoris or Saccharomyces cerevisiae.

In particular embodiments, provided is a method for producing a recombinant heterologous protein in a Pichia pastoris host cell comprising expressing a nucleic acid molecule encoding the recombinant heterologous protein in a recombinant Pichia pastoris host cell in which expression of the endogenous PMT2 gene is disrupted and which comprises a nucleic acid molecule encoding a Pmt2p protein comprising a substitution of the phenylalanine residue at position 664 of the Pmt2p protein with an serine residue to produce the recombinant heterologous protein.

In particular embodiments, provided is a method for producing a recombinant heterologous protein in a Saccharomyces cerevisiae host cell comprising expressing a nucleic acid molecule encoding the recombinant heterologous protein in a recombinant Saccharomyces cerevisiae host cell in which expression of the endogenous PMT2 gene is disrupted and which comprises a nucleic acid molecule encoding a Pmt2p protein comprising a substitution of the phenylalanine residue at position 666 of the Pmt2p protein with a serine residue to produce the recombinant heterologous protein.

In particular embodiments, provided is a method for producing a recombinant heterologous protein in a Pichia pastoris host cell comprising expressing a nucleic acid molecule encoding the recombinant heterologous protein in a recombinant Pichia pastoris host cell in which expression of the endogenous PMT2 gene is disrupted and which comprises a nucleic acid molecule encoding a Pmt2p protein comprising at least 90%, 95%, 96%, 97%, 98% 99%, or 100% identity to the amino acid sequence of SEQ ID NO:3 with the proviso that the amino acid at position 664 is a serine residue to produce the recombinant heterologous protein.

In particular embodiments, provided is a method for producing a recombinant heterologous protein in a Saccharomyces cerevisiae host cell comprising expressing a nucleic acid molecule encoding the recombinant heterologous protein in a recombinant Saccharomyces cerevisiae host cell in which expression of the endogenous PMT2 gene is disrupted and which comprises a nucleic acid molecule encoding a Pmt2p protein comprising at least 90%, 95%, 96%, 97%, 98% 99%, or 100% identity to the amino acid sequence of SEQ ID NO:7 with the proviso that the amino acid at position 666 is a serine residue to produce the recombinant heterologous protein.

In further embodiments, any one of the above host cells may further include a reduction, disruption, or deletion of the function or expression of at least one endogenous PMT gene selected from PMT1, PMT3, PMT4 and PMT6. In particular aspects, the above host cell comprises a deletion or disruption of the PMT4 gene.

In further embodiments, the host cell further includes a nucleic acid molecule encoding an α-1,2-mannosidase that targets the secretory pathway and is secreted by the host cell. In particular aspects, a chimeric α-1,2-mannosidase is provided wherein the catalytic domain of the α-1,2-mannosidase is fused to a heterologous targeting peptide that targets the chimeric α-1,2-mannosidase to the secretory pathway and the chimeric α-1,2-mannosidase is secreted from the host cell. In particular aspects, the α-1,2-mannosidase is from Trichoderma reesei, Saccharomyces sp., or Aspergillus sp. In further aspects, the targeting peptide is Saccharomyces cerevisiae alpha-mating factor pre-signal peptide. In this embodiment, the α-1,2-mannosidase or chimeric α-1,2-mannosidase, which is secreted, reduces the chain length of any O-glycans that may be present even in host cells having any combination of the above mutations and/or deletions to about one mannose residue per O-glycan.

In a further embodiment of the above method, the host cells are grown in the presence of a Pmtp inhibitor for a time sufficient to produce the recombinant heterologous protein. Pmtp inhibitors include but are not limited to a benzylidene thiazolidinediones such as those disclosed in U.S. Pat. No. 7,105,554 and U.S. Published Application No. 20110076721. Examples of benzylidene thiazolidinediones that can be used are 5-[[3,4-bis(phenylmethoxy)phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic Acid.

Thus, the present invention further provides a method for producing a recombinant heterologous protein in a lower eukaryote comprising (a) providing a recombinant lower eukaryote host cell in which expression of the endogenous PMT2 gene is disrupted and which comprises a first nucleic acid molecule encoding a Pmt2p protein comprising an amino acid substitution, deletion, or insertion in an amino acid sequence of the Pmt2p comprising a conserved region having at least 80%, 90%, or 95% identity to the amino acid sequence of SEQ ID NO:9, and a second nucleic acid molecule encoding a recombinant heterologous protein; and (b) growing the host cell in a medium comprising a Pmtp inhibitor for a time sufficient to produce the recombinant heterologous protein.

In particular embodiments, provided is a method for producing a recombinant heterologous protein in a lower eukaryote comprising (a) providing a recombinant lower eukaryote host cell in which expression of the endogenous PMT2 gene is disrupted and which comprises a first nucleic acid molecule encoding a Pmt2p protein comprising a conserved region having at least 80%, 90%, or 95% identity to the amino acid sequence of SEQ ID NO:9 in which the phenylalanine residue at position two of SEQ ID NO:9 is substituted with a serine residue, and a second nucleic acid molecule encoding a recombinant heterologous protein; and (b) growing the host cell in a medium comprising a Pmtp inhibitor for a time sufficient to produce the recombinant heterologous protein.

In particular embodiments, provided is a method for producing a recombinant heterologous protein in a lower eukaryote comprising (a) providing a recombinant lower eukaryote host cell in which expression of the endogenous PMT2 gene is disrupted and which comprises a first nucleic acid molecule encoding a Pmt2p protein comprising at least 95% identity to the amino acid sequence of SEQ ID NO:9 in which the phenylalanine residue at position two of SEQ ID NO:9 is substituted with a serine residue, and a second nucleic acid molecule encoding a recombinant heterologous protein; and (b) growing the host cell in a medium comprising a Pmtp inhibitor for a time sufficient to produce the recombinant heterologous protein.

In particular embodiments, provided is a method for producing a recombinant heterologous protein in a lower eukaryote comprising (a) providing a recombinant lower eukaryote host cell in which expression of the endogenous PMT2 gene is disrupted and which comprises a first nucleic acid molecule encoding a Pmt2p protein comprising at least 98% identity to the amino acid sequence of SEQ ID NO:9 in which the phenylalanine residue at position two of SEQ ID NO:9 is substituted with a serine residue, and a second nucleic acid molecule encoding a recombinant heterologous protein; and (b) growing the host cell in a medium comprising a Pmtp inhibitor for a time sufficient to produce the recombinant heterologous protein.

In particular embodiments, provided is a method for producing a recombinant heterologous protein in a lower eukaryote comprising (a) providing a recombinant lower eukaryote host cell in which expression of the endogenous PMT2 gene is disrupted and which comprises a first nucleic acid molecule encoding a Pmt2p protein comprising an amino acid substitution, deletion, or insertion in the highly conserved region comprising the SEQ ID NO:9, and a second nucleic acid molecule encoding a recombinant heterologous protein; and (b) growing the host cell in a medium comprising a Pmtp inhibitor for a time sufficient to produce the recombinant heterologous protein.

In particular embodiments, provided is a method for producing a recombinant heterologous protein in a lower eukaryote comprising (a) providing a recombinant lower eukaryote host cell in which expression of the endogenous PMT2 gene is disrupted and which comprises a first nucleic acid molecule encoding a Pmt2p protein comprising the amino acid sequence of SEQ ID NO:9 in which the phenylalanine residue at position two of SEQ ID NO:9 is substituted with a serine residue, and a second nucleic acid molecule encoding a recombinant heterologous protein; and (b) growing the host cell in a medium comprising a Pmtp inhibitor for a time sufficient to produce the recombinant heterologous protein.

In particular aspects of the above, the host cell is selected from the group consisting of Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Ogataea minuta, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, and Neurospora crassa. Various yeasts, such as Ogataea minuta, Kluyveromyces lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale.

In further still aspects, the host cell is deficient in the activity of one or more enzymes selected from the group consisting of mannosyltransferases and phosphomannosyltransferases. In further still aspects, the host cell does not express an enzyme selected from the group consisting of 1,6 mannosyltransferase, 1,3 mannosyltransferase, and 1,2 mannosyltransferase.

In a particular aspect of any one of the above host cells, the host cell is Pichia pastoris or Saccharomyces cerevisiae. In a further aspect, the host cell is an och1 mutant of Pichia pastoris or Saccharomyces cerevisiae.

In particular embodiments, provided is a method for producing a recombinant heterologous protein in a Pichia pastoris comprising (a) providing a recombinant Pichia pastoris host cell in which expression of the endogenous PMT2 gene is disrupted and which comprises a first nucleic acid molecule encoding a Pmt2p protein comprising a substitution of the phenylalanine residue at position 664 of the Pmt2p protein with an serine residue, and a second nucleic acid molecule encoding a recombinant heterologous protein; and (b) growing the host cell in a medium comprising a Pmtp inhibitor for a time sufficient to produce the recombinant heterologous protein.

In particular embodiments, provided is a method for producing a recombinant heterologous protein in a Saccharomyces cerevisiae comprising (a) providing a recombinant Saccharomyces cerevisiae host cell in which expression of the endogenous PMT2 gene is disrupted and which comprises a first nucleic acid molecule encoding a Pmt2p protein comprising a substitution of the phenylalanine residue at position 666 of the Pmt2p protein with a serine residue, and a second nucleic acid molecule encoding a recombinant heterologous protein; and (b) growing the host cell in a medium comprising a Pmtp inhibitor for a time sufficient to produce the recombinant heterologous protein.

In particular embodiments, provided is a method for producing a recombinant heterologous protein in a Pichia pastoris comprising (a) providing a recombinant Pichia pastoris host cell in which expression of the endogenous PMT2 gene is disrupted and which comprises a first nucleic acid molecule encoding a Pmt2p protein having at least 90%, 95%, 96%, 97%, 98% 99%, 100% identity to the amino acid sequence of SEQ ID NO:3 with the proviso that the amino acid at position 664 is a serine residue, and a second nucleic acid molecule encoding a recombinant heterologous protein; and (b) growing the host cell in a medium comprising a Pmtp inhibitor for a time sufficient to produce the recombinant heterologous protein.

In particular embodiments, provided is a method for producing a recombinant heterologous protein in a Saccharomyces cerevisiae comprising (a) providing a recombinant Saccharomyces cerevisiea host cell in which expression of the endogenous PMT2 gene is disrupted and which comprises a first nucleic acid molecule encoding a Pmt2p protein having at least 90%, 95%, 96%, 97%, 98% 99%, 100% identity to the amino acid sequence of SEQ ID NO:7 with the proviso that the amino acid at position 666 is a serine residue, and a second nucleic acid molecule encoding a recombinant heterologous protein; and (b) growing the host cell in a medium comprising a Pmtp inhibitor for a time sufficient to produce the recombinant heterologous protein.

In further embodiments, any one of the above host cells may further include a reduction, disruption, or deletion of the function or expression of at least one endogenous PMT gene selected from PMR1, PMT3, PMT4 and PMT6. In particular aspects, the above host cell comprises a deletion or disruption of the PMT4 gene.

In further embodiments, the host cell further includes a nucleic acid molecule encoding an α-1,2-mannosidase that targets the secretory pathway and is secreted by the host cell. In particular aspects, a chimeric α-1,2-mannosidase is provided wherein the catalytic domain of the α-1,2-mannosidase is fused to a heterologous targeting peptide that targets the chimeric α-1,2-mannosidase to the secretory pathway and the chimeric α-1,2-mannosidase is secreted from the host cell. In particular aspects, the α-1,2-mannosidase is from Trichoderma reesei, Saccharomyces sp., or Aspergillus sp. In further aspects, the targeting peptide is Saccharomyces cerevisiae alpha-mating factor pre-signal peptide. In this embodiment, the α-1,2-mannosidase or chimeric α-1,2-mannosidase, which is secreted, reduces the chain length of any O-glycans that may be present even in host cells having any combination of the above mutations and/or deletions to about one mannose residue per O-glycan.

The recombinant host cells may further include any combination of the following genetic manipulations to provide host cells that are capable of expressing glycoproteins in which the N-glycosylation pattern is mammalian-like or human-like or humanized or where a particular N-glycan species is predominant. This may be achieved by eliminating selected endogenous glycosylation enzymes and/or supplying exogenous enzymes as described by Gerngross et al., U.S. Pat. No. 7,449,308, the disclosure of which is incorporated herein by reference, and general methods for reducing O-glycosylation in yeast have been described in International Application No. WO2007061631. In this manner, glycoprotein compositions can be produced in which a specific desired glycoform is predominant in the composition. If desired, additional genetic engineering of the glycosylation can be performed, such that the glycoprotein can be produced with or without core fucosylation. Use of lower eukaryotic host cells such as yeast are further advantageous in that these cells are able to produce relatively homogenous compositions of glycoprotein, such that the predominant glycoform of the glycoprotein may be present as greater than thirty mole percent of the glycoprotein in the composition. In particular aspects, the predominant glycoform may be present in greater than forty mole percent, fifty mole percent, sixty mole percent, seventy mole percent and, most preferably, greater than eighty mole percent of the glycoprotein present in the composition. Such can be achieved by eliminating selected endogenous glycosylation enzymes and/or supplying exogenous enzymes as described by Gerngross et al., U.S. Pat. No. 7,029,872 and U.S. Pat. No. 7,449,308, the disclosures of which are incorporated herein by reference. For example, a host cell can be selected or engineered to be depleted in α1,6-mannosyl transferase activities, which would otherwise add mannose residues onto the N-glycan on a glycoprotein. For example, in yeast such an α1,6-mannosyl transferase activity is encoded by the OCH1 gene and deletion or disruption of the OCH1 inhibits the production of high mannose or hypermannosylated N-glycans in yeast such as Pichia pastoris or Saccharomyces cerevisiae. (See for example, Gerngross et al. in U.S. Pat. No. 7,029,872; Contreras et al. in U.S. Pat. No. 6,803,225; and Chiba et al. in EP1211310B1 the disclosures of which are incorporated herein by reference).

In one embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform. For example, U.S. Pat. No. 7,029,872, U.S. Pat. No. 7,449,308, and U.S. Published Patent Application No. 2005/0170452, the disclosures of which are all incorporated herein by reference, disclose lower eukaryote host cells capable of producing a glycoprotein comprising a Man₅GlcNAc₂ glycoform.

In a further embodiment, the immediately preceding host cell further includes an N-acetylglucosaminyltransferase I (GlcNAc transferase I or GnT I) catalytic domain fused to a cellular targeting signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example a recombinant glycoprotein composition comprising predominantly a GlcNAcMan₅GlcNAc₂ glycoform. U.S. Pat. No. 7,029,872, U.S. Pat. No. 7,449,308, and U.S. Published Patent Application No. 2005/0170452, the disclosures of which are all incorporated herein by reference, disclose lower eukaryote host cells capable of producing a glycoprotein comprising a GlcNAcMan₅GlcNAc₂ glycoform. The glycoprotein produced in the above cells can be treated in vitro with a hexaminidase to produce a recombinant glycoprotein comprising a Man₅GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Pat. No. 7,625,756, the disclosures of which are all incorporated herein by reference, discloses lower eukaryote host cells that express mannosidase II enzymes and are capable of producing glycoproteins having predominantly a GlcNAcMan₃GlcNAc₂ glycoform. The glycoprotein produced in the above cells can be treated in vitro with a hexosaminidase that removes the terminal GlcNAc residue to produce a recombinant glycoprotein comprising a Man₃GlcNAc₂ glycoform or the hexosaminidase can be co-expressed with the glycoprotein in the host cell to produce a recombinant glycoprotein comprising a Man₃GlcNAc₂ glycoform.

In a further embodiment, the immediately preceding host cell further includes N-acetylglucosaminyltransferase II (GlcNAc transferase II or GnT II) catalytic domain fused to a cellular targeting signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. U.S. Pat. Nos. 7,029,872 and 7,449,308 and U.S. Published Patent Application No. 2005/0170452, the disclosures of which are all incorporated herein by reference, disclose lower eukaryote host cells capable of producing a glycoprotein comprising a GlcNAc₂Man₃GlcNAc₂ glycoform. The glycoprotein produced in the above cells can be treated in vitro with a hexosaminidase that removes the terminal GlcNAc residues to produce a recombinant glycoprotein comprising a Man₃GlcNAc₂ glycoform or the hexosaminidase can be co-expressed with the glycoprotein in the host cell to produce a recombinant glycoprotein comprising a Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂Man₃GlcNAc₂ or Gal₂GlcNAc₂Man₃GlcNAc₂ glycoform, or mixture thereof for example a recombinant glycoprotein composition comprising predominantly a GalGlcNAc₂Man₃GlcNAc₂ glycoform or Gal₂GlcNAc₂Man₃GlcNAc₂ glycoform or mixture thereof. U.S. Pat. No. 7,029,872 and U.S. Published Patent Application No. 2006/0040353, the disclosures of which are incorporated herein by reference, discloses lower eukaryote host cells capable of producing a glycoprotein comprising a Gal₂GlcNAc₂Man₃GlcNAc₂ glycoform. The glycoprotein produced in the above cells can be treated in vitro with a galactosidase to produce a recombinant glycoprotein comprising a GlcNAc₂Man₃GlcNAc₂ glycoform, for example a recombinant glycoprotein composition comprising predominantly a GlcNAc₂Man₃GlcNAc₂ glycoform or the galactosidase can be co-expressed with the glycoprotein in the host cell to produce a recombinant glycoprotein comprising the GlcNAc₂Man₃GlcNAc₂ glycoform, for example a recombinant glycoprotein composition comprising predominantly a GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialyltransferase activity to the ER or Golgi apparatus of the host cell. Passage of the recombinant glycoprotein through the ER or Golgi apparatus of the host cell produces a recombinant glycoprotein comprising predominantly a Sia₂Gal₂GlcNAc₂Man₃GlcNAc₂ glycoform or SiaGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Published Patent Application No. 2005/0260729, the disclosure of which is incorporated herein by reference, discloses a method for genetically engineering lower eukaryotes to have a CMP-sialic acid synthesis pathway and U.S. Published Patent Application No. 2006/0286637, the disclosure of which is incorporated herein by reference, discloses a method for genetically engineering lower eukaryotes to produce sialylated glycoproteins. The glycoprotein produced in the above cells can be treated in vitro with a neuraminidase to produce a recombinant glycoprotein comprising predominantly a Gal₂GlcNAc₂Man₃GlcNAc₂ glycoform or GalGlcNAc₂Man₃GlcNAc₂ glycoform or mixture thereof or the neuraminidase can be co-expressed with the glycoprotein in the host cell to produce a recombinant glycoprotein comprising predominantly a Gal₂GlcNAc₂Man₃GlcNAc₂ glycoform or GalGlcNAc₂Man₃GlcNAc₂ glycoform or mixture thereof.

In a further aspect, the above host cell capable of making glycoproteins having a Man₅GlcNAc₂ glycoform can further include a mannosidase III catalytic domain fused to a cellular targeting signal peptide not normally associated with the catalytic domain and selected to target the mannosidase III activity to the ER or Golgi apparatus of the host cell. Passage of the recombinant glycoprotein through the ER or Golgi apparatus of the host cell produces a recombinant glycoprotein comprising a Man₃GlcNAc₂ glycoform, for example a recombinant glycoprotein composition comprising predominantly a Man₃GlcNAc₂ glycoform. U.S. Pat. No. 7,625,756, the disclosures of which are all incorporated herein by reference, discloses the use of lower eukaryote host cells that express mannosidase III enzymes and are capable of producing glycoproteins having predominantly a Man₃GlcNAc₂ glycoform.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Pat. No. 7,598,055 and U.S. Published Patent Application No. 2007/0037248, the disclosures of which are all incorporated herein by reference.

In further embodiments, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising predominantly the GalGlcNAcMan₅GlcNAc₂ glycoform.

In a further embodiment, the immediately preceding host cell that produced glycoproteins that have predominantly the GalGlcNAcMan₅GlcNAc₂ N-glycans further includes a sialyltransferase catalytic domain fused to a cellular targeting signal peptide not normally associated with the catalytic domain and selected to target sialytransferase activity to the ER or Golgi apparatus of the host cell. Passage of the recombinant glycoprotein through the ER or Golgi apparatus of the host cell produces a recombinant glycoprotein comprising a SiaGalGlcNAcMan₅GlcNAc₂ glycoform.

In general yeast and filamentous fungi are not able to make glycoproteins that have N-glycans that include fucose. Therefore, the N-glycans disclosed herein will lack fucose unless the host cell is specifically modified to include a pathway for synthesizing GDP-fucose and a fucosyltransferase. Therefore, in particular aspects where it is desirable to have glycoproteins in which the N-glycan includes fucose, any one of the aforementioned host cells is further modified to include a fucosyltransferase and a pathway for producing fucose and transporting fucose into the ER or Golgi. Examples of methods for modifying Pichia pastoris to render it capable of producing glycoproteins in which one or more of the N-glycans thereon are fucosylated are disclosed in Published International Application No. WO 2008112092, the disclosure of which is incorporated herein by reference. In particular aspects of the invention, the Pichia pastoris host cell is further modified to include a fucosylation pathway comprising a GDP-mannose-4,6-dehydratase, GDP-keto-deoxy-mannose-epimerase/GDP-keto-deoxy-galactose-reductase, GDP-fucose transporter, and a fucosyltransferase. In particular aspects, the fucosyltransferase is selected from the group consisting of α1,2-fucosyltransferase, α1,3-fucosyltransferase, α1,4-fucosyltransferase, and α1,6-fucosyltransferase.

Various of the preceding host cells further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

Host cells further include Pichia pastoris that are genetically engineered to eliminate glycoproteins having phosphomannose residues by deleting or disrupting one or both of the phosphomannosyltransferase genes PNO1 and MNN4B (See for example, U.S. Pat. Nos. 7,198,921 and 7,259,007; the disclosures of which are all incorporated herein by reference), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. The host cells can further include any one of the aforementioned host cells modified to produce particular N-glycan structures.

To reduce or eliminate the likelihood of N-glycans and O-glycans with (3-linked mannose residues, which are resistant to α-mannosidases, the recombinant glycoengineered Pichia pastoris host cells are genetically engineered to eliminate glycoproteins having α-mannosidase-resistant N-glycans by deleting or disrupting one or more of the (3-mannosyltransferase genes (e.g., BMT1, BMT2, BMT3, and BMT4)(See, U.S. Pat. No. 7,465,577, U.S. Pat. No. 7,713,719, and Published International Application No. WO2011046855, each of which is incorporated herein by reference). The deletion or disruption of BMT2 and one or more of BMT1, BMT3, and BMT4 also reduces or eliminates detectable cross reactivity to antibodies against host cell protein.

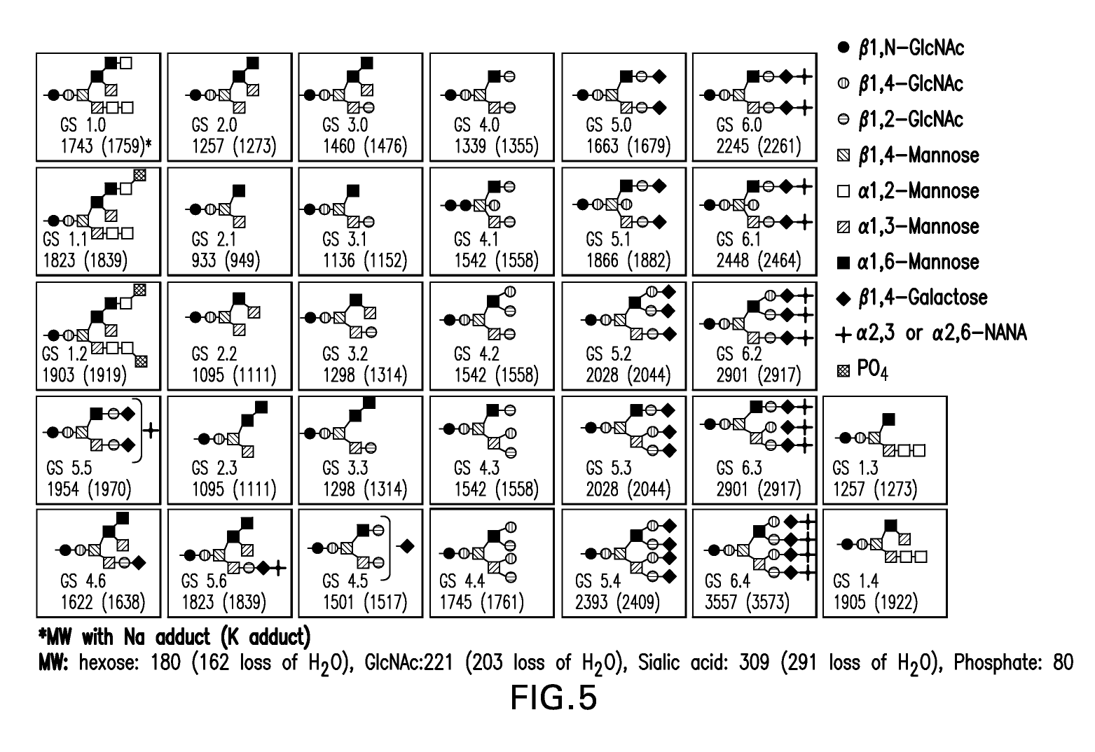

In particular embodiments, the host cells do not display Alg3p protein activity or have a disruption of expression from the ALG3 gene as described in Published U.S. Application No. 20050170452 or US20100227363, which are incorporated herein by reference. Alg3p is Man₅GlcNAc₂-PP-dolichyl alpha-1,3 mannosyltransferase that transferase a mannose residue to the mannose residue of the alpha-1,6 arm of lipid-linked Man₅GlcNAc₂ (FIG. 5, GS 1.3) in an alpha-1,3 linkage to produce lipid-linked Man₆GlcNAc₂ (FIG. 5, GS 1.4), a precursor for the synthesis of lipid-linked Glc₃Man₉GlcNAc₂, which is then transferred by an oligosaccharyltransferase to an aspargine residue of a glycoprotein followed by removal of the glucose (Glc) residues. In host cells that lack Alg3p protein activity, the lipid-linked Man₅GlcNAc₂ oligosaccharide may be transferred by an oligosaccharyltransferase to an aspargine residue of a glycoprotein. In such host cells that further include an α1,2-mannosidase, the Man₅GlcNAc₂ oligosaccharide attached to the glycoprotein is trimmed to a tri-mannose (paucimannose) Man₃GlcNAc₂ structure (FIG. 5, GS 2.1). The Man₅GlcNAc₂ (GS 1.3) structure is distinguishable from the Man₅GlcNAc₂ (GS 2.0) shown in FIG. 5, and which is produced in host cells that express the Man₅GlcNAc₂-PP-dolichyl alpha-1,3 mannosyltransferase (Alg3p).

In further embodiments, the host cell further expresses an endomannosidase activity (e.g., a full-length endomannosidase or a chimeric endomannosidase comprising an endomannosidase catalytic domain fused to a cellular targeting signal peptide not normally associated with the catalytic domain and selected to target the endomannosidase activity to the ER or Golgi apparatus of the host cell. See for example, U.S. Pat. No. 7,332,299) and/or glucosidase II activity (a full-length glucosidase II or a chimeric glucosidase II comprising a glucosidase II catalytic domain fused to a cellular targeting signal peptide not normally associated with the catalytic domain and selected to target the glucosidase II activity to the ER or Golgi apparatus of the host cell. See for example, U.S. Pat. No. 6,803,225). In particular aspects, the host cell further includes a deletion or disruption of the ALG6 (α1,3-glucosylatransferase) gene (alg6Δ), which has been shown to increase N-glycan occupancy of glycoproteins in alg3Δ host cells (See for example, De Pourcq et al., PloSOne 2012; 7(6):e39976. Epub 2012 Jun. 29, which discloses genetically engineering Yarrowia lipolytica to produce glycoproteins that have Man₅GlcNAc₂ (GS 1.3) or paucimannose N-glycan structures). The nucleic acid sequence encoding the Pichia pastoris ALG6 is disclosed in EMBL database, accession number CCCA38426. In further aspects, the host cell further includes a deletion or disruption of the OCH1 gene (och1Δ).

Yield of glycoprotein can in some situations be improved by overexpressing nucleic acid molecules encoding mammalian or human chaperone proteins or replacing the genes encoding one or more endogenous chaperone proteins with nucleic acid molecules encoding one or more mammalian or human chaperone proteins. In addition, the expression of mammalian or human chaperone proteins in the host cell also appears to control O-glycosylation in the cell. Thus, further included are the host cells herein wherein the function of at least one endogenous gene encoding a chaperone protein has been reduced or eliminated, and a vector encoding at least one mammalian or human homolog of the chaperone protein is expressed in the host cell. Also included are host cells in which the endogenous host cell chaperones and the mammalian or human chaperone proteins are expressed. In further aspects, the lower eukaryotic host cell is a yeast or filamentous fungi host cell. Examples of the use of chaperones of host cells in which human chaperone proteins are introduced to improve the yield and reduce or control O-glycosylation of recombinant proteins has been disclosed in Published International Application No. WO2009105357 and WO2010019487 (the disclosures of which are incorporated herein by reference).

Therefore, the methods disclose herein can use any host cell that has been genetically modified to produce glycoproteins comprising at least N-glycan shown in FIG. 5. The methods disclose herein can use any host cell that has been genetically modified to produce glycoproteins wherein the predominant N-glycan is selected from the group consisting of complex N-glycans, hybrid N-glycans, and high mannose N-glycans wherein complex N-glycans are selected from the group consisting of Man₃GlcNAc₂ (paucimannose), GlcNAc₍₁₋₄₎Man₃ GlcNAc₂, Gal₍₁₋₄₎GlcNAc₍₁₋₄₎Man₃GlcNAc₂, and Sia₍₁₋₄₎Gal₍₁₋₄₎Man₃GlcNAc₂; hybrid N-glycans are selected from the group consisting of GlcNAcMan₅GlcNAc₂, GalGlcNAcMan₅GlcNAc₂, and SiaGalGlcNAcMan₅GlcNAc₂; and high Mannose N-glycans are selected from the group consisting of Man₅GlcNAc₂, (GS 2.0), Man₆GlcNAc₂, Man₇GlcNAc₂, Man₈GlcNAc₂, and Man₉GlcNAc₂. In further embodiments, the host cell produces glycoproteins that have predominantly an N-glycan structure consisting of the Man₅GlcNAc₂ (GS 1.3) structure.

To increase the N-glycosylation site occupancy on a glycoprotein produced in a recombinant host cell, a nucleic acid molecule encoding a heterologous single-subunit oligosaccharyltransferase, which is capable of functionally suppressing a lethal mutation of one or more essential subunits comprising the endogenous host cell hetero-oligomeric oligosaccharyltransferase (OTase) complex, is overexpressed in the recombinant host cell either before or simultaneously with the expression of the glycoprotein in the host cell. The Leishmania major STT3A protein, Leishmania major STT3B protein, and Leishmania major STT3D protein, are single-subunit oligosaccharyltransferases that have been shown to suppress the lethal phenotype of a deletion of the STT3 locus in Saccharomyces cerevisiae (Naseb et al., Molec. Biol. Cell 19: 3758-3768 (2008)). Naseb et al. (ibid.) further showed that the Leishmania major STT3D protein could suppress the lethal phenotype of a deletion of the WBP1, OST1, SWP1, or OST2 loci. Hese et al. (Glycobiology 19: 160-171 (2009)) teaches that the Leishmania major STT3A (STT3-1), STT3B (STT3-2), and ST73D (STT3-4) proteins can functionally complement deletions of the OST2, SWP1, and WBP1 loci. As shown in Published International Application No. WO2011106389, which is incorporated herein by reference in its entirety, the Leishmania major STT3D (LmSTT3D) protein is a heterologous single-subunit oligosaccharyltransferases that is capable of suppressing a lethal phenotype of a Δstt3 mutation and at least one lethal phenotype of a Δwbp1, Δost1, Δswp1, and Δost2 mutation that is shown in the examples herein to be capable of enhancing the N-glycosylation site occupancy of heterologous glycoproteins, for example antibodies, produced by the host cell.

Therefore, in a further aspect of the methods herein, provided are yeast or filamentous fungus host cells genetically engineered to be capable of producing glycoproteins with mammalian- or human-like complex or hybrid N-glycans wherein the host cell further includes a nucleic acid molecule encoding a heterologous single-subunit oligosaccharyltransferase (OTase) complex.

In general, in the above methods and host cells, the single-subunit oligosaccharyltransferase is capable of functionally suppressing the lethal phenotype of a mutation of at least one essential protein of the OTase complex. In further aspects, the essential protein of the OTase complex is encoded by the STT3 locus, WBP1 locus, OST1 locus, SWP1 locus, or OST2 locus, or homologue thereof. In further aspects, the for example single-subunit oligosaccharyltransferase is the Leishmania major STT3D protein.

For genetically engineering yeast, selectable markers can be used to construct the recombinant host cells include drug resistance markers and genetic functions which allow the yeast host cell to synthesize essential cellular nutrients, e.g. amino acids. Drug resistance markers that are commonly used in yeast include chloramphenicol, kanamycin, methotrexate, G418 (geneticin), Zeocin, and the like. Genetic functions that allow the yeast host cell to synthesize essential cellular nutrients are used with available yeast strains having auxotrophic mutations in the corresponding genomic function. Common yeast selectable markers provide genetic functions for synthesizing leucine (LEU2), tryptophan (TRP1 and TRP2), proline (PRO1), uracil (URA3, URA5, URA6), histidine (HIS3), lysine (LYS2), adenine (ADE1 or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al., J. Biol. Chem. 272:30061-30066 (1997)). A number of suitable integration sites include those enumerated in U.S. Pat. No. 7,479,389 (the disclosure of which is incorporated herein by reference) and include homologs to loci known for Saccharomyces cerevisiae and other yeast or fungi. Methods for integrating vectors into yeast are well known (See for example, U.S. Pat. No. 7,479,389, U.S. Pat. No. 7,514,253, U.S. Published Application No. 2009012400, and WO2009/085135; the disclosures of which are all incorporated herein by reference). Examples of insertion sites include, but are not limited to, Pichia ADE genes; Pichia TRP (including TRP1 through TRP2) genes; Pichia MCA genes; Pichia CYM genes; Pichia PEP genes; Pichia PRB genes; and Pichia LEU genes. The Pichia ADE1 and ARG4 genes have been described in Lin Cereghino et al., Gene 263:159-169 (2001) and U.S. Pat. No. 4,818,700 (the disclosure of which is incorporated herein by reference), the HIS5 and TRP1 genes have been described in Cosano et al., Yeast 14:861-867 (1998), HIS4 has been described in GenBank Accession No. X56180.

The transformation of the yeast cells is well known in the art and may for instance be effected by protoplast formation followed by transformation in a manner known per se. The medium used to cultivate the cells may be any conventional medium suitable for growing yeast organisms.

In particular embodiments of any one of the above host cells and methods using the host cells, the recombinant heterologous protein is therapeutic protein or glycoprotein, which in particular embodiments may be for example, selected from the group consisting of erythropoietin (EPO); cytokines such as interferon α, interferon β, interferon γ, and interferon ω; and granulocyte-colony stimulating factor (GCSF); granulocyte macrophage-colony stimulating factor (GM-CSF); coagulation factors such as factor VIII, factor IX, and human protein C; antithrombin III; thrombin; soluble IgE receptor α-chain; immunoglobulins such as IgG, IgG fragments, IgG fusions, and IgM; immunoadhesions and other Fc fusion proteins such as soluble TNF receptor-Fc fusion proteins; RAGE-Fc fusion proteins; interleukins; urokinase; chymase; urea trypsin inhibitor; IGF-binding protein; epidermal growth factor; growth hormone-releasing factor; annexin V fusion protein; angiostatin; vascular endothelial growth factor-2; myeloid progenitor inhibitory factor-1; osteoprotegerin; α-1-antitrypsin; α-feto proteins; DNase II; kringle 3 of human plasminogen; glucocerebrosidase; TNF binding protein 1; follicle stimulating hormone; cytotoxic T lymphocyte associated antigen 4-Ig; transmembrane activator and calcium modulator and cyclophilin ligand; glucagon-like protein 1; insulin, and IL-2 receptor agonist.

In further embodiments of any one of the above host cells, the therapeutic glycoprotein is an antibody, examples of which, include but are not limited to, an anti-Her2 antibody, anti-RSV (respiratory syncytial virus) antibody, anti-TNFα antibody, anti-VEGF antibody, anti-CD3 receptor antibody, anti-CD41 7E3 antibody, anti-CD25 antibody, anti-CD52 antibody, anti-CD33 antibody, anti-IgE antibody, anti-CD11a antibody, anti-EGF receptor antibody, or anti-CD20 antibody.

The following examples are intended to promote a further understanding of the present invention.

### Example 1

To identify Pichia strains more tolerant of or resistant to PMT inhibitors, we randomly mutagenized strain YGLY19376, which is a pmt4Δ host genetically engineered to produce glycoproteins with human-like glycosylation patterns and expressing a recombinant IgG1 antibody, using ultraviolet (UV) irradiation followed by subjecting the mutagenized cells to growth-inhibitory concentrations of a PMT inhibitor. Construction of strain YGLY19376 from wild-type Pichia pastoris is described in example 2.

UV mutagenesis was performed as described by Winston (Curr. Protoc. Mol. Biol. 82:13.3B.1-13.3B.5 (2008)). Briefly, Pichia pastoris strain YGLY19376 (GFI5.0, pmt4Δ, expressing a recombinant IgG1) was grown in 40 mL YSD liquid medium over night at 24° C. Upon reaching an OD₆₀₀ of five, a 10 mL aliquot of culture was transferred into an empty 100 mm sterile Petri dish, and treated, with the lid off, with 12 mJ/cm² of UV irradiation. After the UV treatment, the Petri dish was immediately covered with aluminum foil (to prevent photo-induced DNA repair). The mutagenized cells were allowed to recover at 24° C. for three hours in the dark. Two mL of the recovered YGLY19376 was then centrifuged at 2,000 rpm for five minutes. The cell pellet was then re-suspended in 400 μL 2% BMGY media and subsequently plated onto YSD agar plates containing 2 μg/mL or 4 μg/mL PMTi-4 PMT inhibitor (Example 4 compound of U.S. Published Application No. 20110076721 having the structure

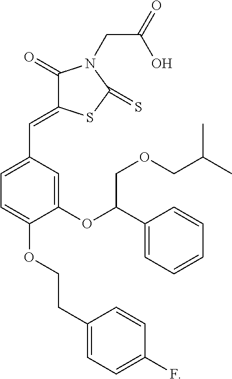 [SMILES: CC(C)COCC(Oc1cc(/C=C2\SC(=S)N(CC(=O)O)C2=O)ccc1OCCc1ccc(F)cc1)c1ccccc1]

After seven days incubation at 24° C., colonies were picked and re-streaked onto fresh PMTi-4-containing plates at the above concentrations. Only those clones that displayed a continued PMT inhibitor-resistance were kept as truly PMT inhibitor-resistant mutants.

### Other Experimental Methods

Fed-batch fermentations, IgG1 purifications, characterization of N- and O-glycan profiles as well as all other analytical assays, were performed essentially as previously described (Barnard et al., J. Ind. Microbiol. Biotechnol. 37(9): 961-71 (2010); Potgieter et al., J. Biotechnol. 139(4):318-25 (2009)).

### Isolation and Characterization of PMTi-Resistant Mutants

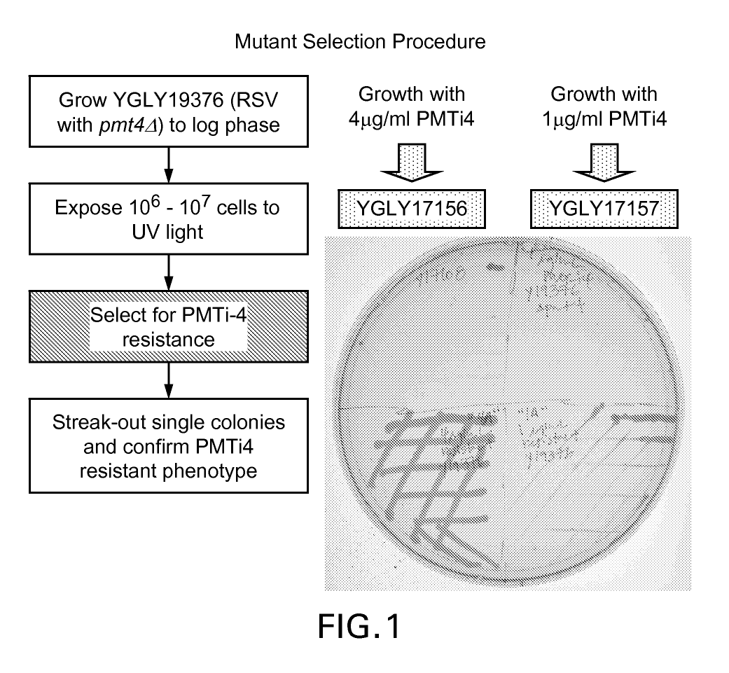

From approximately 10⁷ UV-treated cells, two independent mutant strains were identified: one mutant strain (YGLY17156) exhibited robust growth in the presence of at least about 4 μg/mL PMTi-4; the other mutant strain (YGLY17157) displayed PMTi-resistance of at least about 1 μg/mL level (FIG. 1). As a comparison, the un-mutagenized YGLY19376 parent strain failed to grow under PMT ihibitor concentrations as low as 0.1 μg/mL. FIG. 1 further shows that strain YGLY17156 had greater tolerance to PMT inhibitors that strain YGLY17157.

Having demonstrated that both stains YGLY17156 and YGLY17157 strains acquired mutations leading to their strong PMT inhibitor-resistance phenotypes on agar plates, we next investigated what effects these mutations would have on O-glycosylation and cell robustness during fermentation. Both PMT inhibitor-resistant mutant strains (YGLY17156 and YGLY17157), as well as the non-mutagenized parent strain YGLY19376 were grown in 1 L DasGip fermentors using a standard MeOH fed-batch fermentation protocol (See for example, Potgieter et al., J. Biotechnol. 139(4): 318-25 (2009) and Hopkins et al., Glycobiol. August 12. [Epub ahead of print] PubMed PMID: 21840970 (2011)). These fermentation experiments showed that in the presence of standard PMT-inhibitor concentration (0.5 ml of 1.9 g/L of PMTi-4 stock into 600 ml of culture), both strains YGLY17156 and YGLY17157 exhibited much improved robustness compared to what is observed for non-mutagenized cells genetically engineered to produce glycoproteins with human-like glycosylation patterns.

The robustness of the strains was determined by examining the fermentation cell cultures under microscope. Depending on the proportion of cell debris, a lysis-score was assigned from 0.5 to 5, with 5 being the worst lysis. The mutant strains displayed a lysis-score of one as opposed to a score of three displayed by the non-mutagenized parent strain YGLY19376 at day two of induction. The mutant strains then lasted two more days in MeOH induction and had a lysis score of 1-1.5 when we ended the fermentation after four days' induction (Table 1). In contrast, the non-mutagenized parent strain failed to survive beyond the second day post-induction.

As shown in Table 1, the IgG1 expression titers obtained from both PMT inhibitor-resistant strains were significantly higher than the non-mutagenized parent strain YGLY19376. In the table, WCW refers to wet cell weight, which is a measure of cell growth during fermentation.

TABLE 1
PMTi-4-Resistant Mutants in DasGip Fermentor
(increased robustness and good titer)

| | Lysis at 24° C. | | | | WCW |
|---|---|---|---|---|---|
| Strain | Day 1 | Day 2 | Day 3 | Day 4 | Day 4 |
| YGLY19376 | 1/1.5 | 3 | | | |
| YGLY17156 | 0.5 | 1/1.5 | 1 | 1.5 | 236 |
| YGLY17156 | 0.5/1 | 0.5/1 | 0.5/1 | 1/1.5 | 225 |
| no PMTi-4 | | | | | |
| YGLY17157 | 0.5 | 0.5/1 | 1 | 1 | 303 |

| | | | | | Broth |
|---|---|---|---|---|---|
| | Supernatant Fraction Titer | | | | Titer |
| Strain | Day 1 | Day 2 | Day 3 | Day 4 | Day 4 |
| YGLY19376 | | 659 | | | 470* |
| YGLY17156 | | 475 | 727 | 927 | 709 |
| YGLY17156 | | 190 | 323 | 453 | 351 |
| no PMTi-4 | | | | | |
| YGLY17157 | | 616 | 869 | 1023 | 713 |

*Broth liter harvested on day 2

Table 2 shows that while in this experiment the mutant strain YGLY17156 appeared to exhibit a slightly higher level of O-glycan occupancy showing about 2.4 mole O-glycan per mole protein as compared with about 1.0 mole O-glycan per mole protein obtained from the non-mutagenized parent strain YGLY19376. However, because the mutant strain is more robust and has greater tolerance to PMT inhibitors, higher doses of the PMT inhibitor may reduce the O-glycan occupancy for strain YGLY17156 to the levels similar to those observed for the non-mutagenized parent strain. The O-glycan occupancy observed for mutant strain YGLY17157 was comparable to that of strain YGLY19376.

Table 2 shows that in the absence of any PMT inhibitor-supplementation, the recombinant IgG1 purified from strain YGL17156 contained seven moles O-glycans per mole of H₂L₂ antibody (Table 2) whereas regular PMT inhibitor-sensitive strains typically secretes IgG1 with greater than 20 moles O-glycans per mole of H₂L₂ antibody. However, compared with fermentation in the presence of PMT inhibitor, omitting the inhibitor from the fermentation resulted in approximately a two-fold reduction in IgG1 titer.

Both PMT inhibitor-resistant mutants displayed more favorable N-glycan profiles. As shown in Table 2, the N-glycan profiles for the mutant strains showed lower Man₅GlcNAc_{2 M}5) N-glycan. The parent strain had been genetically engineered to produce galactose-terminated complex N-glycans. With the two PMTi-4 resistant mutants, the amount of complex N-glycans (G0+G1+G2) was greater than the amounts observed for the non-mutagenized parent strain. In the parent strain the amount of G0+G1+G2N-glycans was about 76.1 mole %. However, in YGLY17156, the amount of G0+G1+G2N-glycans was 86.6 mole % and was about 90.2 mole % when the strain was cultivated in the absence of a PMT inhibitor.

TABLE 2
Glycans of PMTi-4-Resistant Mutants from DasGip Fermentor

| | O-Glycan Profiles | | | | |
|---|---|---|---|---|---|
| | Occupancy | O-glycan chain length | | | |
| | (mannitol/protein | M1 | M2 | M3 | M4 |
| Strain | mol/mol) | mol % | Mol % | Mol % | Mol % |
| YGLY19376 | 1.0 | 100 | 0 | 0 | 0 |
| YGLY17156 | 2.4 | 100 | 0 | 0 | 0 |
| YGLY17156 | 7.0 | 100 | 0 | 0 | 0 |
| no PMTi-4 | | | | | |
| YGLY17157 | 0.6 | 100 | 0 | 0 | 0 |

| | N-Glycan Profiles (mole %) | | | | | |
|---|---|---|---|---|---|---|
| Strain | G0 | M5 | G1 | G2 | GNM5 | GalGNM5 |
| YGLY19376 | 48.0 | 19.7 | 17.3 | 10.8 | 0.9 | 3.3 |
| YGLY17156 | 59.9 | 7.1 | 24.7 | 5.6 | 1.2 | 1.5 |
| YGLY17156 | 60.1 | 10.3 | 20.7 | 5.8 | 1.1 | 2.0 |
| no PMTi-4 | | | | | | |
| YGLY17157 | 56.9 | 8.1 | 27.5 | 4.4 | 1.5 | 1.6 |

M1 - one mannose residue
M2 - two mannose residues (mannobiose)
M3 - three mannose residues (mannotriose)
M4 - four mannose residues (mannotetrose)
G0 - GlcNAc₂Man₃GlcNAc₂
M5 - Man₅GlcNAc₂
G1 - GalGlcNAc₂Man₃GlcNAc₂
G2 - Gal₂GlcNAc₂Man₃GlcNAc₂
GNM5 - GlcNAcMan₅GlcNAc₂
GalGNM5 - GalGlcNAcMan₅GlcNAc₂

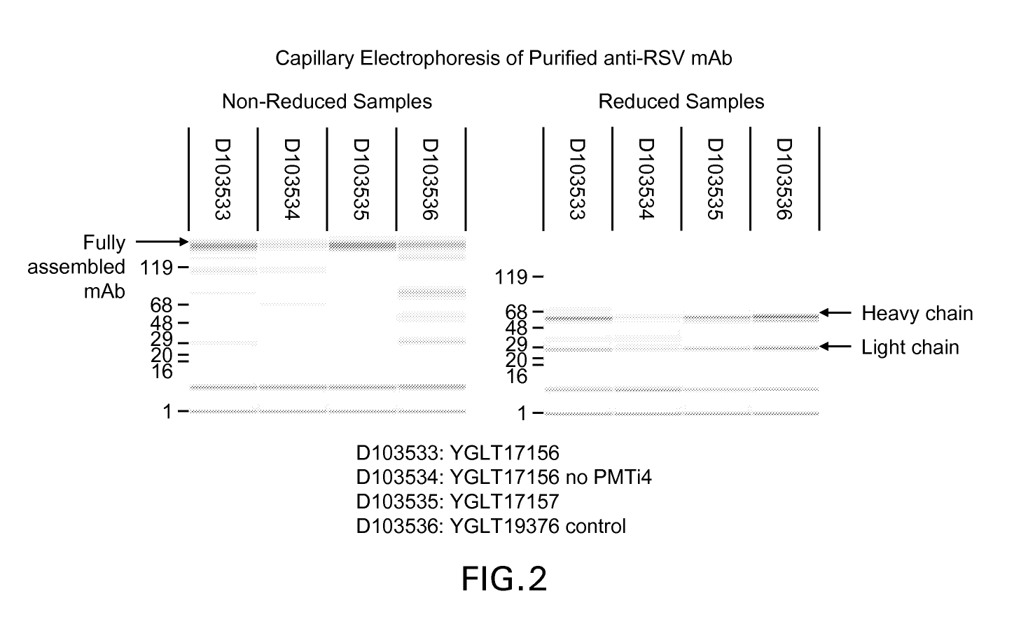

It has been reported that the presence of O-glycans may negatively affect the assembly of recombinant IgG1s in yeast (See for example, Kuroda et al., Appl Environ. Microbiol. 74(2):446-53 2008). To examine IgG1 assembly, purified recombinant IgG1s were resolved by capillary electrophoresis. FIG. 2 shows that the IgG1s from the two mutant strains displayed higher levels of fully-assembled IgG1 (the 150 kb top band) than that obtained from the non-mutagenized parent strain YGLY19376. The results indicated that the mutations harbored in strains YGLY17156 and YGLY17157 did not negatively or adversely impact the IgG1 assembly during its secretion process.

Collectively, these results indicate that the two PMT inhibitor-tolerant mutant strains displayed enhanced fermentation robustness in the presence of PMT inhibitors, low levels of O-glycan occupancy even without PMT inhibitor supplementation, and favorable effects on IgG1 titer, assembly, and N-glycan profiles. These are all very desirable attributes for Pichia hosts that are to be used for the production of recombinant therapeutic proteins.

### Example 2

The parent strain YGLY19376 in Example 1 was constructed from wild-type Pichia pastoris strain NRRL-Y 11430 using methods described earlier (See for example, U.S. Pat. No. 7,449,308; U.S. Pat. No. 7,479,389; U.S. Published Application No. 20090124000; Published PCT Application No. WO2009085135; Nett and Gerngross, Yeast 20:1279 (2003); Choi et al., Proc. Natl. Acad. Sci. USA 100:5022 (2003); Hamilton et al., Science 301:1244 (2003)). All plasmids were made in a pUC19 plasmid using standard molecular biology procedures. For nucleotide sequences that were optimized for expression in P. pastoris, the native nucleotide sequences were analyzed by the GENEOPTIMIZER software (GeneArt, Regensburg, Germany) and the results used to generate nucleotide sequences in which the codons were optimized for P. pastoris expression. Yeast strains were transformed by electroporation (using standard techniques as recommended by the manufacturer of the electroporator BioRad).

From a series of transformations beginning with strain NRRL-Y 11430, strain YGLY8316 was produced. Strain YGLY8316 is capable of producing glycoproteins that have predominately galactose-terminated N-glycans. Construction of this strain from the wild-type NRRL-Y 11430 strain is described in detail in Example 2 of Published International Application No. WO2011106389 and which is incorporated herein by reference.

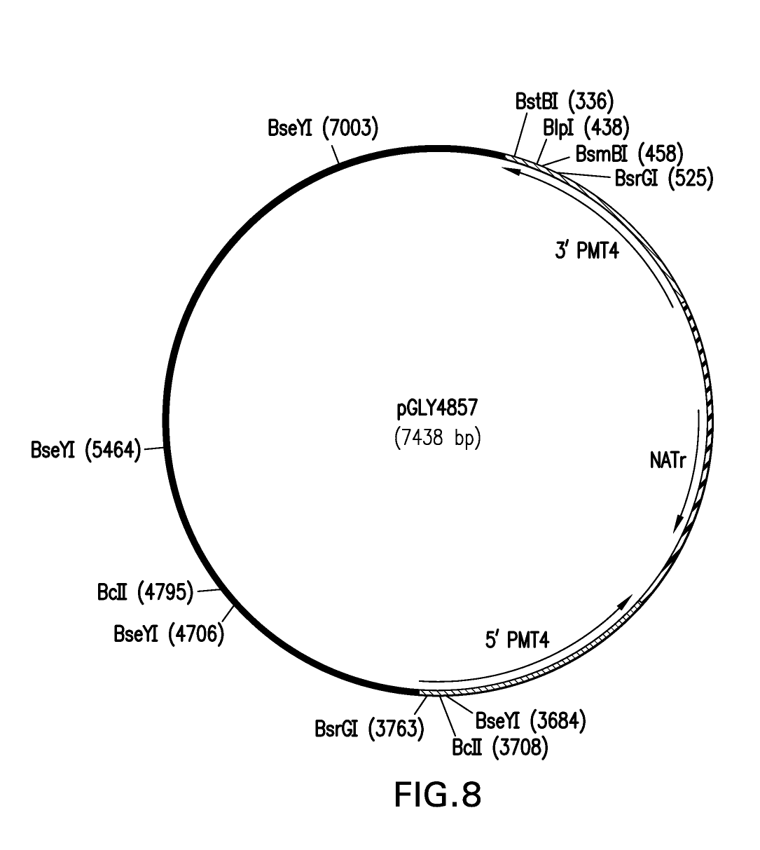

To delete the PMT4 gene in strain YGLY8316, the strain was transformed with plasmid vector pGLY4857, which had been linearized. Plasmid pGLY4857 (FIG. 8) is an integration or knockout vector that targets the PMT4 locus. The vector comprises an expression cassette comprising a nucleic acid molecule (SEQ ID NO:32) encoding the Nourseothricin resistance (NAT^{R}) ORF (originally from pAG25 from EROSCARF, Scientific Research and Development GmbH, Daimlerstrasse 13a, D-61352 Bad Homburg, Germany, See Goldstein et al., Yeast 15: 1541 (1999); GenBank Accession Nos. CAR31387.1 and CAR31383.1) operably linked at the 5′ end to the Ashbya gossypii TEF1 promoter and at the 3′ end to the Ashbya gossypii TEF1 termination sequence. The expression cassette is flanked on one side with the 5′ nucleotide sequence of the P. pastoris PMT4 gene (SEQ ID NO:30) and on the other side with the 3′ nucleotide sequence of the P. pastoris PMT4 gene (SEQ ID NO:31). From the transformation of strain YGLY8316 with plasmid pGLY8316, strain YGLY8795 was selected from the transformants produced.

Strain YGLY8795 was transformed with plasmid pGLY6564 to produce strain YGLY14475, which expresses genes encoding the light and heavy chains of an anti-RSV antibody. Plasmid pGLY6564 is a roll-in integration plasmid encoding the light and heavy chains of an anti-RSV antibody that targets the TRP2 locus in P. pastoris. The expression cassette encoding the anti-RSV heavy chain comprises a nucleic acid molecule encoding the heavy chain ORF codon-optimized for effective expression in P. pastoris (SEQ ID NO:10) operably linked at the 5′ end to a nucleic acid molecule (SEQ ID NO:11) encoding the Saccharomyces cerevisiae mating factor pre-signal sequence which in turn is fused at its N-terminus to a nucleic acid molecule that has the inducible P. pastoris AOX1 promoter sequence (SEQ ID NO:12) and at the 3′ end to a nucleic acid molecule that has the S. cerevisiae CYC transcription termination sequence (SEQ ID NO:13). The expression cassette encoding the anti-RSV light chain comprises a nucleic acid molecule encoding the light chain ORF codon-optimized for effective expression in P. pastoris (SEQ ID NO:14) operably linked at the 5′ end to a nucleic acid molecule encoding the Saccharomyces cerevisiae mating factor pre-signal sequence (SEQ ID NO:11) which in turn is fused at its N-terminus to a nucleic acid molecule that has the inducible P. pastoris AOXI promoter sequence (SEQ ID NO:12) and at the 3′ end to a nucleic acid molecule that has the P. pastoris AOX1 transcription termination sequence (SEQ ID NO:16). For selecting transformants, the plasmid comprises an expression cassette encoding the Zeocin ORF in which the nucleic acid molecule encoding the ORF (SEQ ID NO:15) is operably linked at the 5′ end to a nucleic acid molecule having the S. cerevisiae TEF promoter sequence (SEQ ID NO:17) and at the 3′ end to a nucleic acid molecule having the S. cerevisiae CYC transcription termination sequence (SEQ ID NO:13). The plasmid further includes a nucleic acid molecule for targeting the TRP2 locus (SEQ ID NO:18).

Strain YGLY14475 was generated by transforming pGLY6564, which encodes the anti-RSV antibody, into YGLY8795. The strain YGLY14475 was selected from the strains produced. In this strain, the expression cassettes encoding the anti-RSV heavy and light chains are targeted to the Pichia pastoris TRP2 locus (PpTRP2).

Strain YGLY14475 was grown on agar plate in the present of PMTi-3 at a concentration of 1 μg/mL. PMTi-3 is (5-[[3-(1-Phenyl-2-hydroxy)ethoxy)-4-(2-phenylethoxy)]phenyl]methylene]-4-oxo-2-thioxo-3-thiazolidineacetic Acid), (U.S. Pat. No. 7,105,554; U.S. Published application No. 20090170159). After about seven days of incubation, PMTi-3-resistant colonies started spontaneously to appear. Strain YGLY19376 was selected from the spontaneous colonies showing PMTi-3 resistance and used in the UV treatment to produce strains YGLY17156 and YGLY17157.

### Example 3

In this example the underlying nucleotide alterations that are responsible for the PMT inhibitor-resistant phenotype were identified.

The PMTi-4 inhibitor used to produce the mutant strains in Example 1 is a close chemical analogue of the rhodanine-3-acetic acid derivatives that were originally identified as potent in vitro inhibitors of the Candidas albican Pmt1p protein (U.S. Pat. No. 7,105,554; U.S. Published Application No. 20110076721). Since then, it has been shown that in Saccharomyces cerevisiae these rhodanine-3-acetic acid derivatives also inhibited Pmtp proteins encoded by other PMT genes, for example, Pmt2p, Pmt4p, and Pmt6p (Arroyo et al., Mol. Microbiol. 79(6): 1529-1546 (2011)).

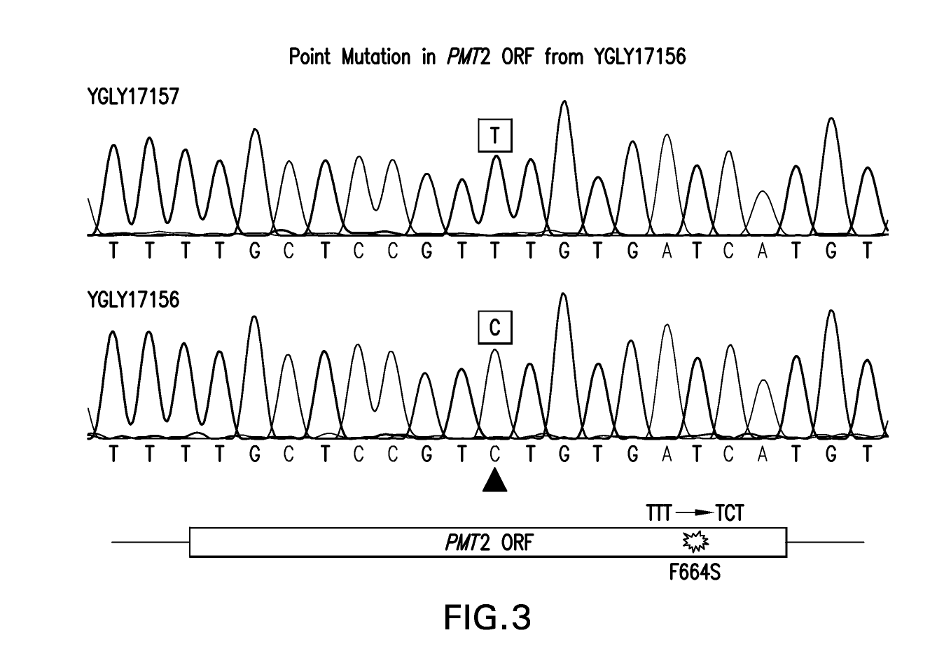

To examine if any of the PMT genes in our PMT inhibitor-resistant mutants were mutated by the UV treatment, the PMT1, PMT2, and PMT6 genes were PCR-amplified from the genomic DNA isolated from the mutant strains YGLY17156 and YGLY17157 (the PMT4 gene has been previously deleted from the non-mutagenized parent strain YGLY19376, hence there was no need to PCR-amplify the PMT4 gene). The PCR-amplified nucleotide sequences encoding the respective Pmtp protein were sequenced. After sequencing the nucleotide sequences encoding the ORFs for each of the three PMT genes, one point mutation was found within the PMT2 gene of strain YGLY17156 (FIG. 3). The observed PMT2 mutation was a “T” to a “C” nucleotide transition 1991 bp downstream of the ATG start codon. This nucleotide mutation led to an amino acid change at position 664 from phenylalanine encoded by TTT to serine encoded by TCT (the “F664S” point mutation). Position 664 is located within a highly conserved region close to the C-terminus of the Pmt2p protein (FIGS. 4 and 5). No nucleotide changes were found in the PMT1 and PMT6 genes from strain YGLY17156. While YGLY17157 is also PMT inhibitor-resistant, the nucleotide sequences for the PMT1, PMT2 and PMT6 genes were all indistinguishable from the nucleotide sequences from the non-mutagenized parent strain. Thus, the observed PMT inhibitor-resistance of strain YGLY17157 is by a mutation that is distinguishable from the resistance due to the PMT2 mutation identified in strain YGLY17156.

### Example 4

This example shows that the F664S mutation in the mutant Pmt2p protein confers the observed PMT inhibitor-resistance. Plasmid vector pGLY5931 was constructed to determine whether the identified PMT2-T1991C point-mutation encoding the Pmt2p-F664S mutant protein was responsible for the observed PMT inhibitor-resistance phenotype.

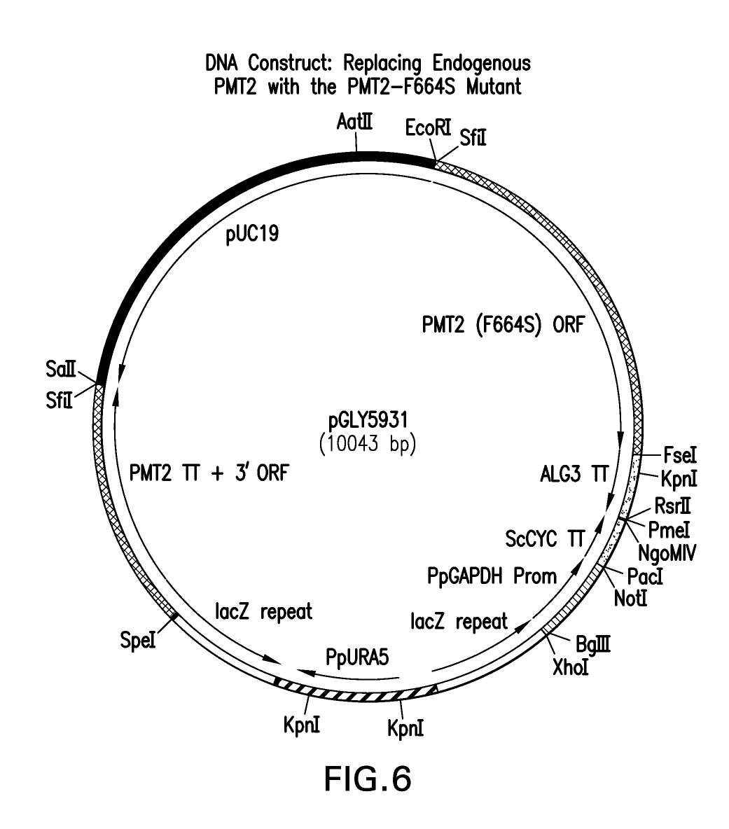

Plasmid pGLY5931 (FIG. 6) is an integration vector that targets the PMT2 locus and contains a nucleic acid molecule comprising the P. pastoris URA5 gene or transcription unit (SEQ ID NO:19) flanked by nucleic acid molecules comprising lacZ repeats (SEQ ID NO:20) which in turn is flanked on one side by a nucleic acid molecule comprising a nucleotide sequence containing the ORF encoding the mutant Pmtp2 gene (F664S mutant) (SEQ ID NO:4) and on the other side by a nucleic acid molecule comprising a nucleotide sequence from the 3′ region of the PMT2 gene (SEQ ID NO:21). This plasmid vector is designed to replace the open reading frame (ORF) encoding the wild-type Pmt2p with the Pmt2p-F664S mutant protein. When the plasmid vector is transformed into a PMT inhibitor-sensitive strain, the vector will precisely delete the wild-type PMT2 ORF and insert the ORF encoding the Pmt2p-F664S mutant protein in its place. The vector also includes the URA5 gene, which enables selection of Ura+ recombinants when transformed into a strain auxotrophic for uracil.

Plasmid pGLY5931 (encoding the Pmt2p-F664S mutant protein) was linearized with SfiI and the linearized plasmid transformed into several PMT inhibitor-sensitive host cells: strain YGLY19313 (expressing an anti-Her2 antibody), strain YGLY8458 (empty host), and strain YGLY9884 (empty host with pmt4Δ deletion). These strains had also been genetically engineered to produce galactose-terminated complex N-glycans. The transformation produced a number of strains prototrophic for uracil and in which the URA5 gene flanked by the lacZ repeats had been inserted into the PMT2 locus by double-crossover homologous recombination. This replaces the nucleotide sequence encoding the endogenous Pmt2p protein with the nucleotide sequence encoding the Pmt2p-F664S mutant protein.

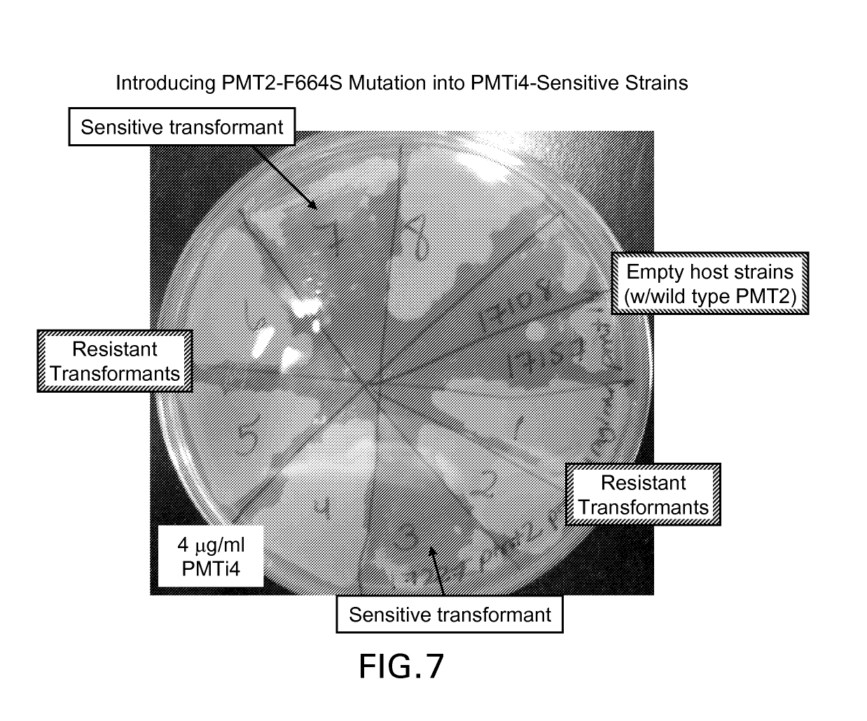

Following the transformation, we observed that the targeted replacement of the wild-type ORF encoding the endogenous Pmt2p protein with the ORF encoding the Pmt2p-F664S mutant protein changed the PMT inhibitor-sensitive strains into strains that were resistant to the PMT inhibitor. FIG. 7 shows that when eight transformants were plated on to agar plates containing 4 μg/mL PMTi-4, six of the transformants displayed a phenotype resistant to up to at least 4 μg/mL PMTi-4.

These results show that the PMT2-T1991C point mutation, which results in Pmt2p-F664S mutant protein, was sufficient to confer the PMT inhibitor-resistant phenotype to the transformant. Furthermore, the discovery of a PMT2 point-mutation causing PMT inhibitor-resistance provides experimental evidence for a direct interaction between the PMT inhibitor and the Pmt2p protein, most likely within the conserved region that includes the amino acid residue at position 664.

Because this point mutation may be introduced into any Pichia pastoris strain and render the recipient strain PMT inhibitor-resistant, the PMT2-T 1991C point mutation, which results in Pmt2p-F664S mutant protein, replacing the endogenous PMT2 gene with a gene encoding a Pmt2p-F664S mutant protein has a broad utility for any heterologous protein-expressing yeast host strain where desired attributes are: reduction in protein O-glycan occupancy; increased PMTi-tolerance; and increased strain robustness and viability during fermentation.

### Example 5

The DasGip Protocol for growing the recombinant host cells is substantially as follows.

The inoculum seed flasks were inoculated from yeast patches (isolated from a single colony) on agar plates into 0.1 L of 4% BSGY in a 0.5-L baffled flask. Seed flasks were grown at 180 rpm and 24° C. (Innova 44, New Brunswick Scientific) for 48 hours. Cultivations were done in 1 L (fedbatch-pro, DASGIP BioTools) bioreactors. Vessels were charged with 0.54 L of 0.22 μm filtered 4% BSGY media and autoclaved at 121° C. for 45 minutes. After sterilization and cooling; the aeration, agitation and temperatures were set to 0.7 vvm, 400 rpm and 24° C. respectively. The pH was adjusted to and controlled at 6.5 using 30% ammonium hydroxide. Inoculation of a prepared bioreactor occurred aseptically with 60 mL from a seed flask. Agitation was ramped to maintain 20% dissolved oxygen (DO) saturation. After the initial glycerol charge was consumed, denoted by a sharp increase in the dissolved oxygen, a 50% w/w glycerol solution containing 5 mg/L biotin and 32.3 mg/L PMTi4 was triggered to feed at 3.68 mL/hr for eight hours. During the glycerol fed-batch phase 0.375 mL of PTM2 salts were injected manually. Completion of the glycerol fed-batch was followed by a 0.5 hour starvation period and initiation of the induction phase. A continuous feed of a 50% v/v methanol solution containing 2.5 mg/L biotin and 6.25 mL/L PTM2 salts was started at a flat rate of 2.16 mL/hour. Injections of 0.25 mL of 1.9 mg/mL PMTi-4 (in methanol) were added after each 24 hours of induction. In general, individual fermentations were harvested within 36-110 hours of induction. The culture broth was clarified by centrifugation (Sorvall Evolution RC, Thermo Scientific) at 8500 rpm for 40 min and the resulting supernatant was submitted for purification.

4% BSGY with 100 mM Sorbitol

| Component | Concentration (g/L) |
|---|---|
| KH₂PO₄ (monobasic) | 11.9 |
| K₂HPO₄ (dibasic) | 2.5 |
| Sorbitol | 18.2 |
| Yeast Extract | 10 |
| Soytone | 20 |
| Glycerol | 40 |
| YNB | 13.4 |
| Biotin | 20 (ml/L) |
| Anti-foam | 8 drops/L* |

Solution to be autoclaved once made

PTM2 Salts

| Component | Concentration (g/L) |
|---|---|
| CuSO₄—5H₂O | 1.50 |
| NaI | 0.08 |
| MnSO₄—H₂O | 1.81 |
| H₃BO₄ | 0.02 |
| FeSO₄—7H₂O | 6.50 |
| ZnC₁₂ | 2.00 |
| CoC₁₂—6H₂O | 0.50 |
| Na₂MoO₄—2H₂O | 0.20 |
| Biotin (dry stock) | 0.20 |
| 98% H₂SO₄ | 5 mL/L |

Dissolve in 80% of the desired total volume of DI water.
Once dissolved make up to final total volume with DI water
Filter under vacuum through 0.22 micron filter into sterile bottle.
Label with Solution Name, Batch Number, and Date. Store at 4° C.

Table of Sequences

| SEQ | | |
|---|---|---|
| ID NO | Description | Sequence |
| 1 | PpPMT2 | MTGRVDQKSDQKVKELIEKIDSESTSRVFQEEPVTSILTRYEPYVAPIIFTLLSFFT |
| | wt | RMYKIGINNHVVWDEAHFGKFGSYYLRHEFYHDVHPPLGKMLVGLSGYIAGYNGSWD |
| | | FPSGQEYPDYIDYVKMRLFNATFSALCVPFAYFTMKEIGFDIKTTWLFTLMVLCETS |
| | | YCTLGKFILLDSMLLLFTVTTVFTFVRFHNENSKPGNSFSRKWWKWLLLTGISIGLT |
| | | CSVKMVGLFVTVLVGIYTVVDLWNKFGDQSISRKKYAAHWLARFIGLIAIPIGVFLL |
| | | SFRIHFEILSNSGTGDANMSSLFQANLRGSSVGGGPRDVTTLNSKVTIKSQGLGSGL |
| | | LHSHVQTYPQGSSQQQITTYSHKDANNDWVFQLTREDSRNAFKEAHYVVDGMSVRLV |
| | | HSNTGRNLHTHQVAAPVSSSEWEVSCYGNETIGDPKDNWIVEIVDQYGDEDKLRLHP |
| | | LTSSFRLKSATLGCYLGTSGASLPQWGFRQGEVVCYKNPFRRDKRTWWNIEDHNNPD |
| | | LPNPPENFVLPRTHFLKDFVQLNLAMMATNNALVPDPDKEDNLASSAWEWPTLHVGI |
| | | RLCGWGDDNVKYFLIGSPATTWTSSVGIVVFLFLLLIYLIKWQRQYVIFPSVQTPLE |
| | | SADTKTVALFDKSDSFNVFLMGGLYPLLGWGLHFAPFVIMSRVTYVHHYLPALYFAM |
| | | IVFCYLVSLLDKKLGHPALGLLIYVALYSLVIGTFIWLSPVVFGMDGPNRNYSYLNL |
| | | LPSWRVSDP |
| 2 | DNA | atgacaggccgtgtcgaccagaaatctgatcagaaggtgaaggaattgatcgaaaag |
| | encodes | atcgactccgaatccacttccagagtttttcaggaagaaccagtcacttcgatcttg |
| | PpPMT2 | acacgttacgaaccctatgtcgccccaattatattcacgttgttgtcctttttcact |
| | wt | cgtatgtacaaaattgggatcaacaaccacgtcgtttgggatgaagctcacttcgga |
| | | aagtttggctcctactatctcagacacgagttctaccacgatgtccaccctccgttg |
| | | ggtaagatgttggtcggtctatctggctacattgccggttacaatggctcctgggat |
| | | ttcccctccggtcaagagtaccctgactatattgattacgttaaaatgaggttattc |
| | | aatgccaccttcagtgccttatgtgtgccattcgcctatttcaccatgaaggagatt |
| | | ggatttgatatcaagacaacttggctattcacactgatggtcttgtgtgaaacaagt |
| | | tattgtacgttaggaaaattcatcttgctggattcaatgctgctgctattcactgtg |
| | | actacggttttcacctttgttaggttccataacgaaaacagtaaaccaggaaactcg |
| | | ttttctcgcaaatggtggaaatggcttctgcttactggtatttccattggtctcact |
| | | tgttccgtcaaaatggtgggtttatttgtcacagtattagttggaatttacacagtt |
| | | gttgacttatggaataaatttggtgatcaatccatttctcgtaagaaatatgctgct |
| | | cattggctagctcgtttcatcggcttgattgccatcccaattggcgtttttctattg |
| | | tcattccgtatccattttgaaatattatccaattctggtaccggtgatgcaaacatg |
| | | tcttcattgttccaagctaaccttcgtggatcatccgtcggaggaggccccagagat |
| | | gtgaccactctcaactctaaagtgaccataaagagccaaggtttaggatctggtctg |
| | | ttacattcccacgttcaaacttatcctcaaggttccagccaacaacagattacaacc |
| | | tattctcacaaagatgccaacaatgattgggtgtttcaacttacgagagaagactct |
| | | cgaaacgctttcaaggaagcccactatgtcgttgatggtatgtctgttcgtctcgtt |
| | | cattcaaacactggtagaaacttacacactcaccaagttgctgctcccgtctcctca |
| | | tccgaatgggaagtcagttgttatggtaatgaaaccattggagacccgaaagataat |
| | | tggattgttgaaattgtcgaccagtatggtgatgaagataagctgagattgcaccca |
| | | ttgacctccagtttccgtttgaaatcggcaactctgggatgctatttgggtacttcg |
| | | ggtgcttcactgcctcaatggggtttcagacaaggtgaagttgtttgttacaaaaat |
| | | ccgttccgtagagataagcgcacctggtggaacatcgaggaccataacaatcctgat |
| | | ctacctaatcctccagaaaattttgttcttcccaggactcattttttgaaagacttt |
| | | gttcaattaaatttagcaatgatggcaacaaacaacgctttggtcccagacccagat |
| | | aaggaagataatctagcttcttctgcctgggaatggcccacgctacacgttggtatc |
| | | cgtctgtgcggttggggcgatgacaacgtcaagtatttcttgattggttctcccgca |
| | | accacctggacttcttcagttggtattgtagtattcctgttcctgctgttaatttac |
| | | ttgatcaaatggcaacgtcaatatgtcattttcccatccgtccagactccactagag |
| | | tcagccgacaccaaaacagttgcattgtttgacaagtctgatagcttcaacgtcttc |
| | | cttatgggaggattatacccgcttctgggatggggtttacattttgctccgtttgtg |
| | | atcatgtcgcgtgttacctacgttcaccattatcttcctgcattgtactttgccatg |
| | | attgttttctgctacttggtttctctgttggataagaaactaggccacccagcatta |
| | | ggattactgatctatgtggctctgtattccttggtcattggaacatttatttggctc |
| | | agccccgttgtgtttggtatggacggtccgaacagaaattacagttacctaaacctt |
| | | ctacctagttggagagtatcagaccca |
| 3 | PpPMT2 | MTGRVDQKSDQKVKELIEKIDSESTSRVFQEEPVTSILTRYEPYVAPIIFTLLSFFT |
| | (F664S) | RMYKIGINNHVVWDEAHFGKFGSYYLRHEFYHDVHPPLGKMLVGLSGYIAGYNGSWD |
| | | FPSGQEYPDYIDYVKMRLFNATFSALCVPFAYFTMKEIGFDIKTTWLFTLMVLCETS |
| | | YCTLGKFILLDSMLLLFTVTTVFTFVRFHNENSKPGNSFSRKWWKWLLLTGISIGLT |
| | | CSVKMVGLFVTVLVGIYTVVDLWNKFGDQSISRKKYAAHWLARFIGLIAIPIGVFLL |
| | | SFRIHFEILSNSGTGDANMSSLFQANLRGSSVGGGPRDVTTLNSKVTIKSQGLGSGL |
| | | LHSHVQTYPQGSSQQQITTYSHKDANNDWVFQLTREDSRNAFKEAHYVVDGMSVRLV |
| | | HSNTGRNLHTHQVAAPVSSSEWEVSCYGNETIGDPKDNWIVEIVDQYGDEDKLRLHP |
| | | LTSSFRLKSATLGCYLGTSGASLPQWGFRQGEVVCYKNPFRRDKRTWWNIEDHNNPD |
| | | LPNPPENFVLPRTHFLKDFVQLNLAMMATNNALVPDPDKEDNLASSAWEWPTLHVGI |
| | | RLCGWGDDNVKYFLIGSPATTWTSSVGIVVFLFLLLIYLIKWQRQYVIFPSVQTPLE |
| | | SADTKTVALFDKSDSFNVFLMGGLYPLLGWGLHFAPSVIMSRVTYVHHYLPALYFAM |
| | | IVFCYLVSLLDKKLGHPALGLLIYVALYSLVIGTFIWLSPVVFGMDGPNRNYSYLNL |
| | | LPSWRVSDP |
| 4 | DNA | atgacaggccgtgtcgaccagaaatctgatcagaaggtgaaggaattgatcgaaaag |
| | encodes | atcgactccgaatccacttccagagtttttcaggaagaaccagtcacttcgatcttg |
| | PmPMT2 | acacgttacgaacCctatgtcgccccaattatattcacgttgttgtcctttttcact |
| | (F664S) | cgtatgtacaaaattgggatcaacaaccacgtcgtttgggatgaagctcacttcgga |
| | | aagtttggctectactatctcagacacgagttctaccacgatgtccaccctccgttg |
| | | ggtaagatgttggtcggtctatctggctacattgccggttacaatggctcctgggat |
| | | ttcccctccggtcaagagtaccctgactatattgattacgttaaaatgaggttattc |
| | | aatgccaccttcagtgccttatgtgtgccattcgcctatttcaccatgaaggagatt |
| | | ggatttgatatcaagacaacttggctattcacactgatggtcttgtgtgaaacaagt |
| | | tattgtacgttaggaaaattcatcttgctggattcaatgctgctgctattcactgtg |
| | | actacggttttcacctttgttaggttccataacgaaaacagtaaaccaggaaactcg |
| | | ttttctcgcaaatggtggaaatggcttctgcttactggtatttccattggtctcact |
| | | tgttccgtcaaaatggtgggtttatttgtcacagtattagttggaatttacacagtt |
| | | gttgacttatggaataaatttggtgatcaatccatttctcgtaagaaatatgctgct |
| | | cattggctagctcgtttcatcggcttgattgccatcccaattggcgtttttctattg |
| | | tcattccgtatccattttgaaatattatccaattctggtaccggtgatgcaaacatg |
| | | tcttcattgttccaagctaaccttcgtggatcatccgtcggaggaggccccagagat |
| | | gtgaccactctcaactctaaagtgaccataaagagccaaggtttaggatctggtctg |
| | | ttacattcccacgttcaaacttatcctcaaggttccagccaacaacagattacaacc |
| | | tattctcacaaagatgccaacaatgattgggtgtttcaacttacgagagaagactct |
| | | cgaaacgctttcaaggaagcccactatgtcgttgatggtatgtctgttcgtctcgtt |
| | | cattcaaacactggtagaaacttacacactcaccaagttgctgctcccgtctcctca |
| | | tccgaatgggaagtcagttgttatggtaatgaaaccattggagacccgaaagataat |
| | | tggattgttgaaattgtcgaccagtatggtgatgaagataagctgagattgcaccca |
| | | ttgacctccagtttccgtttgaaatcggcaactctgggatgctatttgggtacttcg |
| | | ggtgcttcactgcctcaatggggtttcagacaaggtgaagttgtttgttacaaaaat |
| | | ccgttccgtagagataagcgcacctggtggaacatcgaggaccataacaatcctgat |
| | | ctacctaatcctccagaaaattttgttcttcccaggactcattttttgaaagacttt |
| | | gttcaattaaatttagcaatgatggcaacaaacaacgctttggtcccagacccagat |
| | | aaggaagataatctagcttcttctgcctgggaatggcccacgctacacgttggtatc |
| | | cgtctgtgcggttggggcgatgacaacgtcaagtatttcttgattggttctcccgca |
| | | accacctggacttcttcagttggtattgtagtattcctgttcctgctgttaatttac |
| | | ttgatcaaatggcaacgtcaatatgtcattttcccatccgtccagactccactagag |
| | | tcagccgacaccaaaacagttgcattgtttgacaagtctgatagcttcaacgtcttc |
| | | cttatgggaggattatacccgcttctgggatggggtttacattttgctccgtctgtg |
| | | atcatgtcgcgtgttacctacgttcaccattatcttcctgcattgtactttgccatg |
| | | attgttttctgctacttggtttctctgttggataagaaactaggccacccagcatta |
| | | ggattactgatctatgtggctctgtattccttggtcattggaacatttatttggctc |
| | | agccccgttgtgtttggtatggacggtccgaacagaaattacagttacctaaacctt |
| | | ctacctagttggagagtatcagaccca |
| 5 | ScPMT2 | MSSSSSTGYSKNNAAHIKQENTLRQRESSSISVSEELSSADERDAEDFSKEKPAAQS |
| | wt | SLLRLESVVMPVIFTALALFTRMYKIGINNHVVWDEAHFGKFGSYYLRHEFYHDVHP |
| | | PLGKMLVGLSGYLAGYNGSWDFPSGEIYPDYLDYVKMRLFNASFSALCVPLAYFTAK |
| | | AIGFSLPTVWLMTVLVLFENSYSTLGRFILLDSMLLFFTVASFFSFVMFHNQRSKPF |
| | | SRKWWKWLLITGISLGCTISVKMVGLFIITMVGIYTVIDLWTFLADKSMSWKTYINH |
| | | WLARIFGLIIVPFCIFLLCFKIHFDLLSHSGTGDANMPSLFQARLVGSDVGQGPRDI |
| | | ALGSSVVSIKNQALGGSLLHSHIQTYPDGSNQQQVTCYGYKDANNEWFFNRERGLPS |
| | | WSENETDIEYLKPGTSYRLVHKSTGRNLHTHPVAAPVSKTQWEVSGYGDNVVGDNKD |
| | | NWVIEIMDQRGDEDPEKLHTLTTSFRIKNLEMGCYLAQTGNSLPEWGFRQQEVVCMK |
| | | NPFKRDKRTWWNIETHENERLPPRPEDFQYPKTNFLKDFIHLNLAIAMATNNALVPD |
| | | PDKFDYLASSAWQWPTLNVGLRLCGWGDDNPKYFLLGTPASTWASSVAVLAFMATVV |
| | | ILLIRWQRQYVDLRNPSNWNVFLMGGFYPLLAWGLHYMPFVIMSRVTYVHHYLPALY |
| | | FALIILAYCFDAGLQKWSRSKCGRIMRFVLYAGFMALVIGCFWYFSPISFGMEGPSS |
| | | NFRYLNWFSTWDIADKQEA |
| 6 | DNA | atgtcctcgtcttcgtctaccgggtacagcaaaaacaatgccgcccacattaagcaa |
| | encodes | gagaatacactgagacaaagagaatcgtcttccatcagcgtcagtgaggaactttcg |
| | ScPMT2 | agcgctgatgagagagacgcggaagatttctcgaaggaaaagcccgctgcacaaagc |
| | wt | tcactgttacgcctggaatccgttgtaatgccggtgatctttactgcattggcgttg |
| | | tttaccaggatgtacaaaatcggcatcaacaaccatgttgtttgggatgaggcgcac |
| | | tttggtaaatttggttcttattacttgagacacgaattttaccacgatgtccatcct |
| | | cccctaggaaaaatgctggtcgggttgtctggttatttggcaggctacaacggttct |
| | | tgggacttcccttctggggaaatttacccagactatttggattatgttaaaatgaga |
| | | ctgttcaacgcgtcattttccgcgctctgtgtgccattggcctacttcactgccaaa |
| | | gctattggattttctttaccaacagtttggctgatgaccgtgttggttttgtttgaa |
| | | aactcgtatagtactttgggcaggttcattcttttggactccatgctacttttcttc |
| | | actgtcgcatcgttctttagttttgttatgttccacaaccagaggtccaagccgttc |
| | | tctagaaagtggtggaaatggctgttgatcactggtatttctttgggttgcactatt |
| | | tccgtcaaaatggtgggtctatttatcatcactatggtcggtatctatactgtgatt |
| | | gacttatggacctttttggcagataaatccatgtcatggaaaacctatattaaccac |
| | | tggttggcaagaatatttggtcttattatcgtccccttctgcattttcctattgtgc |
| | | ttcaaaatacattttgacctattatcgcattctggtacaggtgatgctaacatgcca |
| | | tctcttttccaagcaagattagtgggttctgacgtcggacaaggcccccgtgacatt |
| | | gctctaggttcctccgttgtttccatcaaaaaccaagctcttggaggatctctattg |
| | | cactcacatatacaaacttatccagatgggtccaaccaacaacaagtaacctgttat |
| | | ggttacaaagatgctaacaacgaatggtttttcaacagagaaagaggcttaccatca |
| | | tggtcagaaaacgaaactgacatcgagtatttgaagccaggtacctcctatagattg |
| | | gtacacaaaagcacgggcagaaacttgcacacccacccagttgctgcaccagtgtca |
| | | aagacacaatgggaggtttctggttacggtgacaatgttgttggtgacaacaaagac |
| | | aattgggttattgagatcatggaccaaagaggagatgaagaccctgagaagttgcac |
| | | acattgaccacctctttccgtatcaagaacttggagatgggctgttacttggctcaa |
| | | accggtaacagtttgcccgaatggggtttcagacaacaagaggttgtctgcatgaaa |
| | | aacccattcaagagggacaagaggacctggtggaacatcgagacccacgaaaatgaa |
| | | aggttgccaccaagacccgaagattttcaatacccaaagaccaacttcttaaaagac |
| | | ttcattcatttaaatctagccatgatggccactaataacgctttggtgccagatcca |
| | | gacaaatttgattacttagcttcctcagcatggcaatggccaactttgaatgtgggt |
| | | ttgagactatgtggctggggtgatgataatccaaaatacttcctattgggtacccca |
| | | gcttccacgtgggcttctagtgttgccgtcctcgcattcatggccacggtcgttatc |
| | | ttactgatcagatggcaaagacaatatgtggacctaagaaatccatctaactggaac |
| | | gttttcttaatgggcgggttctacccactactagcttggggcctacactacatgcca |
| | | ttcgttatcatgtctagagtcacctacgttcatcattacttgcctgccttgtatttt |
| | | gcactgatcattttggcgtactgtttcgacgccggtttgcaaaaatggtccagatct |
| | | aagtgcggccgtatcatgcggttcgtcctatacgccggattcatggcacttgtaatt |
| | | ggttgcttctggtacttctccccaatatcatttggtatggagggaccaagtagtaac |
| | | ttccgctacttaaactggttttccacttgggacattgccgacaagcaagaagca |
| 7 | ScPMT2 | MSSSSSTGYSKNNAAHIKQENTLRQRESSSISVSEELSSADERDAEDFSKEKPAAQS |
| | (F666S) | SLLRLESVVMPVIFTALALFTRMYKIGINNHVVWDEAHFGKFGSYYLRHEFYHDVHP |
| | | PLGKMLVGLSGYLAGYNGSWDFPSGEIYPDYLDYVKMRLFNASFSALCVPLAYFTAK |
| | | AIGFSLPTVWLMTVLVLFENSYSTLGRFILLDSMLLFFTVASFFSFVMFHNQRSKPF |
| | | SRKWWKWLLITGISLGCTISVKMVGLFIITMVGIYTVIDLWTFLADKSMSWKTYINH |
| | | WLARIFGLIIVPFCIFLLCFKIHFDLLSHSGTGDANMPSLFQARLVGSDVGQGPRDI |
| | | ALGSSVVSIKNQALGGSLLHSHIQTYPDGSNQQQVTCYGYKDANNEWFFNRERGLPS |
| | | WSENETDIEYLKPGTSYRLVHKSTGRNLHTHPVAAPVSKTQWEVSGYGDNVVGDNKD |
| | | NWVIEIMDQRGDEDPEKLHTLTTSFRIKNLEMGCYLAQTGNSLPEWGFRQQEVVCMK |
| | | NPFKRDKRTWWNIETHENERLPPRPEDFQYPKTNFLKDFIHLNLAMMATNNALVPDP |
| | | DKFDYLASSAWQWPTLNVGLRLCGWGDDNPKYFLLGTPASTWASSVAVLAFMATVVI |
| | | LLIRWQRQYVDLRNPSNWNVFLMGGFYPLLAWGLHYMPSVIMSRVTYVHHYLPALYF |
| | | ALIILAYCFDAGLQKWSRSKCGRIMRFVLYAGFMALVIGCFWYFSPISFGMEGPSSN |
| | | FRYLNWFSTWDIADKQEA |
| 8 | DNA | atgtcctcgtcttcgtctaccgggtacagcaaaaacaatgccgcccacattaagcaa |
| | encoding | gagaatacactgagacaaagagaatcgtcttccatcagcgtcagtgaggaactttcg |
| | ScPMT2 | agcgctgatgagagagacgcggaagatttctcgaaggaaaagcccgctgcacaaagc |
| | (F666S) | tcactgttacgcctggaatccgttgtaatgccggtgatctttactgcattggcgttg |
| | | tttaccaggatgtacaaaatcggcatcaacaaccatgttgtttgggatgaggcgcac |
| | | tttggtaaatttggttcttattacttgagacacgaattttaccacgatgtccatcct |
| | | cccctaggaaaaatgctggtcgggttgtctggttatttggcaggctacaacggttct |
| | | tgggacttcccttctggggaaatttacccagactatttggattatgttaaaatgaga |
| | | ctgttcaacgcgtcattttccgcgctctgtgtgccattggcctacttcactgccaaa |
| | | gctattggattttctttaccaacagtttggctgatgaccgtgttggttttgtttgaa |
| | | aactcgtatagtactttgggcaggttcattcttttggactccatgctacttttcttc |
| | | actgtcgcatcgttctttagttttgttatgttccacaaccagaggtccaagccgttc |
| | | tctagaaagtggtggaaatggctgttgatcactggtatttctttgggttgcactatt |
| | | tccgtcaaaatggtgggtctatttatcatcactatggtcggtatctatactgtgatt |
| | | gacttatggacctttttggcagataaatccatgtcatggaaaacctatattaaccac |
| | | tggttggcaagaatatttggtcttattatcgtccccttctgcattttcctattgtgc |
| | | ttcaaaatacattttgacctattatcgcattctggtacaggtgatgctaacatgcca |
| | | tctcttttccaagcaagattagtgggttctgacgtcggacaaggcccccgtgacatt |
| | | gctctaggttcctccgttgtttccatcaaaaaccaagctcttggaggatctctattg |
| | | cactcacatatacaaacttatccagatgggtccaaccaacaacaagtaacctgttat |
| | | ggttacaaagatgctaacaacgaatggtttttcaacagagaaagaggcttaccatca |
| | | tggtcagaaaacgaaactgacatcgagtatttgaagccaggtacctcctatagattg |
| | | gtacacaaaagcacgggcagaaacttgcacacccacccagttgctgcaccagtgtca |
| | | aagacacaatgggaggtttctggttacggtgacaatgttgttggtgacaacaaagac |
| | | aattgggttattgagatcatggaccaaagaggagatgaagaccctgagaagttgcac |
| | | acattgaccacctctttccgtatcaagaacttggagatgggctgttacttggctcaa |
| | | accggtaacagtttgcccgaatggggtttcagacaacaagaggttgtctgcatgaaa |
| | | aacccattcaagagggacaagaggacctggtggaacatcgagacccacgaaaatgaa |
| | | aggttgccaccaagacccgaagattttcaatacccaaagaccaacttcttaaaagac |
| | | ttcattcatttaaatctagccatgatggccactaataacgctttggtgccagatcca |
| | | gacaaatttgattacttagcttcctcagcatggcaatggccaactttgaatgtgggt |
| | | ttgagactatgtggctggggtgatgataatccaaaatacttcctattgggtacccca |
| | | gcttccacgtgggcttctagtgttgccgtcctcgcattcatggccacggtcgttatc |
| | | ttactgatcagatggcaaagacaatatgtggacctaagaaatccatctaactggaac |
| | | gttttcttaatgggcgggttctacccactactagcttggggcctacactacatgcca |
| | | tccgttatcatgtctagagtcacctacgttcatcattacttgcctgccttgtatttt |
| | | gcactgatcattttggcgtactgtttcgacgccggtttgcaaaaatggtccagatct |
| | | aagtgcggccgtatcatgcggttcgtcctatacgccggattcatggcacttgtaatt |
| | | ggttgcttctggtacttctccccaatatcatttggtatggagggaccaagtagtaac |
| | | ttccgctacttaaactggttttccacttgggacattgccgacaagcaagaagca |
| 9 | PpPmt2p | PFVIMSRVTYVHHYLPALYFA |
| | conserved | |
| | region | |
| 10 | Anti-RSV | CAGGTTACATTGAGAGAATCCGGTCCAGCTTTGGTTAAGCCAACTCAGACTTTGACT |
| | Heavy | TTGACTTGTACTTTCTCCGGTTTCTCCTTGTCTACTTCCGGAATGTCTGTTGGATGG |
| | chain | ATCAGACAACCACCTGGAAAGGCTTTGGAATGGCTTGCTGACATTTGGTGGGATGAC |
| | (VH + IgG1 | AAGAAGGACTACAACCCATCCTTGAAGTCCAGATTGACTATCTCCAAGGACACTTCC |
| | constant | AAGAATCAAGTTGTTTTGAAGGTTACAAACATGGACCCAGCTGACACTGCTACTTAC |
| | region) | TACTGTGCTAGATCCATGATCACTAACTGGTACTTCGATGTTTGGGGTGCTGGTACT |
| | (DNA) | ACTGTTACTGTCTCGAGTGCTTCTACTAAGGGACCATCCGTTTTTCCATTGGCTCCA |
| | | TCCTCTAAGTCTACTTCCGGTGGAACCGCTGCTTTGGGATGTTTGGTTAAAGACTAC |
| | | TTCCCAGAGCCAGTTACTGTTTCTTGGAACTCCGGTGCTTTGACTTCTGGTGTTCAC |
| | | ACTTTCCCAGCTGTTTTGCAATCTTCCGGTTTGTACTCTTTGTCCTCCGTTGTTACT |
| | | GTTCCATCCTCTTCCTTGGGTACTCAGACTTACATCTGTAACGTTAACCACAAGCCA |
| | | TCCAACACTAAGGTTGACAAGAGAGTTGAGCCAAAGTCCTGTGACAAGACACATACT |
| | | TGTCCACCATGTCCAGCTCCAGAATTGTTGGGTGGTCCATCCGTTTTCTTGTTCCCA |
| | | CCAAAGCCAAAGGACACTTTGATGATCTCCAGAACTCCAGAGGTTACATGTGTTGTT |
| | | GTTGACGTTTCTCACGAGGACCCAGAGGTTAAGTTCAACTGGTACGTTGACGGTGTT |
| | | GAAGTTCACAACGCTAAGACTAAGCCAAGAGAAGAGCAGTACAACTCCACTTACAGA |
| | | GTTGTTTCCGTTTTGACTGTTTTGCACCAGGACTGGTTGAACGGTAAAGAATACAAG |
| | | TGTAAGGTTTCCAACAAGGCTTTGCCAGCTCCAATCGAAAAGACTATCTCCAAGGCT |
| | | AAGGGTCAACCAAGAGAGCCACAGGTTTACACTTTGCCACCATCCAGAGAAGAGATG |
| | | ACTAAGAACCAGGTTTCCTTGACTTGTTTGGTTAAAGGATTCTACCCATCCGACATT |
| | | GCTGTTGAGTGGGAATCTAACGGTCAACCAGAGAACAACTACAAGACTACTCCACCA |
| | | GTTTTGGATTCTGATGGTTCCTTCTTCTTGTACTCCAAGTTGACTGTTGACAAGTCC |
| | | AGATGGCAACAGGGTAACGTTTTCTCCTGTTCCGTTATGCATGAGGCTTTGCACAAC |
| | | CACTACACTCAAAAGTCCTTGTCTTTGTCCCCTGGTTAA |
| 11 | Saccharomyces | ATGAGATTCCCATCCATCTTCACTGCTGTTTTGTTCGCTGCTTCTTCTGCTTTGGCT |
| | cerevisiae | |
| | mating | |
| | factor pre- | |
| | signal peptide | |
| | (DNA) | |
| 12 | Pp AOX1 | AACATCCAAAGACGAAAGGTTGAATGAAACCTTTTTGCCATCCGACATCCACAGGTC |
| | promoter | CATTCTCACACATAAGTGCCAAACGCAACAGGAGGGGATACACTAGCAGCAGACCGT |
| | | TGCAAACGCAGGACCTCCACTCCTCTTCTCCTCAACACCCACTTTTGCCATCGAAAA |
| | | ACCAGCCCAGTTATTGGGCTTGATTGGAGCTCGCTCATTCCAATTCCTTCTATTAGG |
| | | CTACTAACACCATGACTTTATTAGCCTGTCTATCCTGGCCCCCCTGGCGAGGTTCAT |
| | | GTTTGTTTATTTCCGAATGCAACAAGCTCCGCATTACACCCGAACATCACTCCAGAT |
| | | GAGGGCTTTCTGAGTGTGGGGTCAAATAGTTTCATGTTCCCCAAATGGCCCAAAACT |
| | | GACAGTTTAAACGCTGTCTTGGAACCTAATATGACAAAAGCGTGATCTCATCCAAGA |
| | | TGAACTAAGTTTGGTTCGTTGAAATGCTAACGGCCAGTTGGTCAAAAAGAAACTTCC |
| | | AAAAGTCGGCATACCGTTTGTCTTGTTTGGTATTGATTGACGAATGCTCAAAAATAA |
| | | TCTCATTAATGCTTAGCGCAGTCTCTCTATCGCTTCTGAACCCCGGTGCACCTGTGC |
| | | CGAAACGCAAATGGGGAAACACCCGCTTTTTGGATGATTATGCATTGTCTCCACATT |
| | | GTATGCTTCCAAGATTCTGGTGGGAATACTGCTGATAGCCTAACGTTCATGATCAAA |
| | | ATTTAACTGTTCTAACCCCTACTTGACAGCAATATATAAACAGAAGGAAGCTGCCCT |
| | | GTCTTAAACCTTTTTTTTTATCATCATTATTAGCTTACTTTCATAATTGCGACTGGT |
| | | TCCAATTGACAAGCTTTTGATTTTAACGACTTTTAACGACAACTTGAGAAGATCAAA |
| | | AAACAACTAATTATTCGAAACG |
| 13 | ScCYC TT | ACAGGCCCCTTTTCCTTTGTCGATATCATGTAATTAGTTATGTCACGCTTACATTCA |
| | | CGCCCTCCTCCCACATCCGCTCTAACCGAAAAGGAAGGAGTTAGACAACCTGAAGTC |
| | | TAGGTCCCTATTTATTTTTTTTAATAGTTATGTTAGTATTAAGAACGTTATTTATAT |
| | | TTCAAATTTTTCTTTTTTTTCTGTACAAACGCGTGTACGCATGTAACATTATACTGA |
| | | AAACCTTGCTTGAGAAGGTTTTGGGACGCTCGAAGGCTTTAATTTGCAAGCTGCCGG |
| | | CTCTTAAG |
| 14 | Anti-RSV | ATGAGATTCCCATCCATCTTCACTGCTGTTTTGTTCGCTGCTTCTTCTGCTTTGGCT |
| | light chain | GACATTCAGATGACACAGTCCCCATCTACTTTGTCTGCTTCCGTTGGTGACAGAGTT |
| | (VL + Kappa | ACTATCACTTGTAAGTGTCAGTTGTCCGTTGGTTACATGCACTGGTATCAGCAAAAG |
| | constant | CCAGGAAAGGCTCCAAAGTTGTTGATCTACGACACTTCCAAGTTGGCTTCCGGTGTT |
| | region | CCATCTAGATTCTCTGGTTCCGGTTCTGGTACTGAGTTCACTTTGACTATCTCTTCC |
| | (DNA) | TTGCAACCAGATGACTTCGCTACTTACTACTGTTTCCAGGGTTCTGGTTACCCATTC |
| | | ACTTTCGGTGGTGGTACTAAGTTGGAGATCAAGAGAACTGTTGCTGCTCCATCCGTT |
| | | TTCATTTTCCCACCATCCGACGAACAATTGAAGTCCGGTACCGCTTCCGTTGTTTGT |
| | | TTGTTGAACAACTTCTACCCACGTGAGGCTAAGGTTCAGTGGAAGGTTGACAACGCT |
| | | TTGCAATCCGGTAACTCCCAAGAATCCGTTACTGAGCAGGATTCTAAGGATTCCACT |
| | | TACTCATTGTCCTCCACTTTGACTTTGTCCAAGGCTGATTACGAGAAGCACAAGGTT |
| | | TACGCTTGCGAGGTTACACATCAGGGTTTGTCCTCCCCAGTTACTAAGTCCTTCAAC |
| | | AGAGGAGAGTGTTAA |
| 15 | Sequence | ATGGCCAAGTTGACCAGTGCCGTTCCGGTGCTCACCGCGCGCGACGTCGCCGGAGCG |
| | of the Sh | GTCGAGTTCTGGACCGACCGGCTCGGGTTCTCCCGGGACTTCGTGGAGGACGACTTC |
| | ble ORF | GCCGGTGTGGTCCGGGACGACGTGACCCTGTTCATCAGCGCGGTCCAGGACCAGGTG |
| | (Zeocin | GTGCCGGACAACACCCTGGCCTGGGTGTGGGTGCGCGGCCTGGACGAGCTGTACGCC |
| | resistance | GAGTGGTCGGAGGTCGTGTCCACGAACTTCCGGGACGCCTCCGGGCCGGCCATGACC |
| | marker): | GAGATCGGCGAGCAGCCGTGGGGGCGGGAGTTCGCCCTGCGCGACCCGGCCGGCAAC |
| | | TGCGTGCACTTCGTGGCCGAGGAGCAGGACTGA |
| 16 | PpAOX1 | TCAAGAGGATGTCAGAATGCCATTTGCCTGAGAGATGCAGGCTTCATTTTGATACTT |
| | TT | TTTTATTTGTAACCTATATAGTATAGGATTTTTTTTGTCATTTTGTTTCTTCTCGTA |
| | | CGAGCTTGCTCCTGATCAGCCTATCTCGCAGCTGATGAATATCTTGTGGTAGGGGTT |
| | | TGGGAAAATCATTCGAGTTTGATGTTTTTCTTGGTATTTCCCACTCCTCTTCAGAGT |
| | | ACAGAAGATTAAGTGAGACGTTCGTTTGTGCA |
| 17 | ScTEF1 | GATCCCCCACACACCATAGCTTCAAAATGTTTCTACTCCTTTTTTACTCTTCCAGAT |
| | promoter | TTTCTCGGACTCCGCGCATCGCCGTACCACTTCAAAACACCCAAGCACAGCATACTA |
| | | AATTTCCCCTCTTTCTTCCTCTAGGGTGTCGTTAATTACCCGTACTAAAGGTTTGGA |
| | | AAAGAAAAAAGAGACCGCCTCGTTTCTTTTTCTTCGTCGAAAAAGGCAATAAAAATT |
| | | TTTATCACGTTTCTTTTTCTTGAAAATTTTTTTTTTTGATTTTTTTCTCTTTCGATG |
| | | ACCTCCCATTGATATTTAAGTTAATAAACGGTCTTCAATTTCTCAAGTTTCAGTTTC |
| | | ATTTTTCTTGTTCTATTACAACTTTTTTTACTTCTTGCTCATTAGAAAGAAAGCATA |
| | | GCAATCTAATCTAAGTTTTAATTACAAA |
| 18 | Sequence | GGTTTCTCAATTACTATATACTACTAACCATTTACCTGTAGCGTATTTCTTTTCCCT |
| | of the | CTTCGCGAAAGCTCAAGGGCATCTTCTTGACTCATGAAAAATATCTGGATTTCTTCT |
| | PpTRP2 | GACAGATCATCACCCTTGAGCCCAACTCTCTAGCCTATGAGTGTAAGTGATAGTCAT |
| | gene | CTTGCAACAGATTATTTTGGAACGCAACTAACAAAGCAGATACACCCTTCAGCAGAA |
| | integration | TCCTTTCTGGATATTGTGAAGAATGATCGCCAAAGTCACAGTCCTGAGACAGTTCCT |
| | locus: | AATCTTTACCCCATTTACAAGTTCATCCAATCAGACTTCTTAACGCCTCATCTGGCT |
| | | TATATCAAGCTTACCAACAGTTCAGAAACTCCCAGTCCAAGTTTCTTGCTTGAAAGT |
| | | GCGAAGAATGGTGACACCGTTGACAGGTACACCTTTATGGGACATTCCCCCAGAAAA |
| | | ATAATCAAGACTGGGCCTTTAGAGGGTGCTGAAGTTGACCCCTTGGTGCTTCTGGAA |
| | | AAAGAACTGAAGGGCACCAGACAAGCGCAACTTCCTGGTATTCCTCGTCTAAGTGGT |
| | | GGTGCCATAGGATACATCTCGTACGATTGTATTAAGTACTTTGAACCAAAAACTGAA |
| | | AGAAAACTGAAAGATGTTTTGCAACTTCCGGAAGCAGCTTTGATGTTGTTCGACACG |
| | | ATCGTGGCTTTTGACAATGTTTATCAAAGATTCCAGGTAATTGGAAACGTTTCTCTA |
| | | TCCGTTGATGACTCGGACGAAGCTATTCTTGAGAAATATTATAAGACAAGAGAAGAA |
| | | GTGGAAAAGATCAGTAAAGTGGTATTTGACAATAAAACTGTTCCCTACTATGAACAG |
| | | AAAGATATTATTCAAGGCCAAACGTTCACCTCTAATATTGGTCAGGAAGGGTATGAA |
| | | AACCATGTTCGCAAGCTGAAAGAACATATTCTGAAAGGAGACATCTTCCAAGCTGTT |
| | | CCCTCTCAAAGGGTAGCCAGGCCGACCTCATTGCACCCTTTCAACATCTATCGTCAT |
| | | TTGAGAACTGTCAATCCTTCTCCATACATGTTCTATATTGACTATCTAGACTTCCAA |
| | | GTTGTTGGTGCTTCACCTGAATTACTAGTTAAATCCGACAACAACAACAAAATCATC |
| | | ACACATCCTATTGCTGGAACTCTTCCCAGAGGTAAAACTATCGAAGAGGACGACAAT |
| | | TATGCTAAGCAATTGAAGTCGTCTTTGAAAGACAGGGCCGAGCACGTCATGCTGGTA |
| | | GATTTGGCCAGAAATGATATTAACCGTGTGTGTGAGCCCACCAGTACCACGGTTGAT |
| | | CGTTTATTGACTGTGGAGAGATTTTCTCATGTGATGCATCTTGTGTCAGAAGTCAGT |
| | | GGAACATTGAGACCAAACAAGACTCGCTTCGATGCTTTCAGATCCATTTTCCCAGCA |
| | | GGAACCGTCTCCGGTGCTCCGAAGGTAAGAGCAATGCAACTCATAGGAGAATTGGAA |
| | | GGAGAAAAGAGAGGTGTTTATGCGGGGGCCGTAGGACACTGGTCGTACGATGGAAAA |
| | | TCGATGGACACATGTATTGCCTTAAGAACAATGGTCGTCAAGGACGGTGTCGCTTAC |
| | | CTTCAAGCCGGAGGTGGAATTGTCTACGATTCTGACCCCTATGACGAGTACATCGAA |
| | | ACCATGAACAAAATGAGATCCAACAATAACACCATCTTGGAGGCTGAGAAAATCTGG |
| | | ACCGATAGGTTGGCCAGAGACGAGAATCAAAGTGAATCCGAAGAAAACGATCAATGA |
| | | ACGGAGGACGTAAGTAGGAATTTATG |
| 19 | Sequence | TCTAGAGGGACTTATCTGGGTCCAGACGATGTGTATCAAAAGACAAATTAGAGTATT |
| | of the | TATAAAGTTATGTAAGCAAATAGGGGCTAATAGGGAAAGAAAAATTTTGGTTCTTTA |
| | PpURA5 | TCAGAGCTGGCTCGCGCGCAGTGTTTTTCGTGCTCCTTTGTAATAGTCATTTTTGAC |
| | auxotrophic | TACTGTTCAGATTGAAATCACATTGAAGATGTCACTGGAGGGGTACCAAAAAAGGTT |
| | marker: | TTTGGATGCTGCAGTGGCTTCGCAGGCCTTGAAGTTTGGAACTTTCACCTTGAAAAG |
| | | TGGAAGACAGTCTCCATACTTCTTTAACATGGGTCTTTTCAACAAAGCTCCATTAGT |
| | | GAGTCAGCTGGCTGAATCTTATGCTCAGGCCATCATTAACAGCAACCTGGAGATAGA |
| | | CGTTGTATTTGGACCAGCTTATAAAGGTATTCCTTTGGCTGCTATTACCGTGTTGAA |
| | | GTTGTACGAGCTGGGCGGCAAAAAATACGAAAATGTCGGATATGCGTTCAATAGAAA |
| | | AGAAAAGAAAGACCACGGAGAAGGTGGAAGCATCGTTGGAGAAAGTCTAAAGAATAA |
| | | AAGAGTACTGATTATCGATGATGTGATGACTGCAGGTACTGCTATCAACGAAGCATT |
| | | TGCTATAATTGGAGCTGAAGGTGGGAGAGTTGAAGGTTGTATTATTGCCCTAGATAG |
| | | AATGGAGACTACAGGAGATGACTCAAATACCAGTGCTACCCAGGCTGTTAGTCAGAG |
| | | ATATGGTACCCCTGTCTTGAGTATAGTGACATTGGACCATATTGTGGCCCATTTGGG |
| | | CGAAACTTTCACAGCAGACGAGAAATCTCAAATGGAAACGTATAGAAAAAAGTATTT |
| | | GCCCAAATAAGTATGAATCTGCTTCGAATGAATGAATTAATCCAATTATCTTCTCAC |
| | | CATTATTTTCTTCTGTTTCGGAGCTTTGGGCACGGCGGCGGATCC |
| 20 | Sequence | CCTGCACTGGATGGTGGCGCTGGATGGTAAGCCGCTGGCAAGCGGTGAAGTGCCTCT |
| | of the part | GGATGTCGCTCCACAAGGTAAACAGTTGATTGAACTGCCTGAACTACCGCAGCCGGA |
| | of the Ec | GAGCGCCGGGCAACTCTGGCTCACAGTACGCGTAGTGCAACCGAACGCGACCGCATG |
| | lacZ gene | GTCAGAAGCCGGGCACATCAGCGCCTGGCAGCAGTGGCGTCTGGCGGAAAACCTCAG |
| | that was used | TGTGACGCTCCCCGCCGCGTCCCACGCCATCCCGCATCTGACCACCAGCGAAATGGA |
| | to construct | TTTTTGCATCGAGCTGGGTAATAAGCGTTGGCAATTTAACCGCCAGTCAGGCTTTCT |
| | the PpURA5 | TTCACAGATGTGGATTGGCGATAAAAAACAACTGCTGACGCCGCTGCGCGATCAGTT |
| | blaster | CACCCGTGCACCGCTGGATAACGACATTGGCGTAAGTGAAGCGACCCGCATTGACCC |
| | (recyclable | TAACGCCTGGGTCGAACGCTGGAAGGCGGCGGGCCATTACCAGGCCGAAGCAGCGTT |
| | auxotrophic | GTTGCAGTGCACGGCAGATACACTTGCTGATGCGGTGCTGATTACGACCGCTCACGC |
| | marker) | GTGGCAGCATCAGGGGAAAACCTTATTTATCAGCCGGAAAACCTACCGGATTGATGG |
| | | TAGTGGTCAAATGGCGATTACCGTTGATGTTGAAGTGGCGAGCGATACACCGCATCC |
| | | GGCGCGGATTGGCCTGAACTGCCAG |
| 21 | Encodes 3′ | ctagtatttaaatgtgtatatatcgtcagtacctaaatttatgatagggtaaaaccg |
| | of Pmt2p | acatcttctatctacaattaatgcgcgactcacgctttctcttatctgcttggtctt |
| | ORF and 3′ | gtttgacttcatcgttagcgttcgcttctctcttcaggtaaaaccttaccctccatg |
| | termination | tcgacctatagagaactggagcgcgaggagggcgacttctctgataagaagcttctc |
| | sequences | acaggctggttctccttaaccgtgggacaactacaatatgctgcattcatggccgta |
| | | ggaatcgcgttgctttggccttggaattgctttctatcagcttcagacttctttgga |
| | | gagcggttgcaagaacacaagtggctctctgctaactattcatcgtccatgatgacc |
| | | atttcaacgttgacctcaacgttatgcaacgtgtttttgtcccaaaagcaaagtggg |
| | | gtagattattcaaagagactcgtcatgggccaaacaatcaccatagtcgtatttgcc |
| | | tttatgggtctgctgtgcgtatggaatacggggctagatccaataatattttttgtg |
| | | ttggtcatgattaatgttgcactgagttcagtagctgtgtcgttatcacaggtcggt |
| | | gctatggcgatcgtgaatgtgttgggaccaatatatgctaatgctgtggttgtgggg |
| | | aatgctgttgctggtgtgctaccgtccatcgctctgattattagcactgccctgtcg |
| | | ggaactcatgtggctggaaagttgcaacctaaaagagattatgcagttatggcatac |
| | | tttttgactgcctgtgtcgttagttggtattgcattagttctttttgggttggcaga |
| | | gtcacatggcccaatcgacgttgttgctgcacctgttcatacttcttctactgctga |
| | | tgaggctattgaagaactaggtatccctttagaagaggaggagtatgttcccttttc |
| | | aactctgtgggccaaacttcgttttgttgcactcacaatatttacagtctttggggt |
| | | ttctttggtcttcccagtctttgcatcgagtattgtttccgccaacggaatcaacag |
| | | tcgcatatttgtccccttggcattcctactctggaaccttggtgatttagcaggtcg |
| | | attactatgtgcttatcccagatttgtcacgcgatctcccataaaattatttatttt |
| | | ctccttagcaagatttctttacatcccgctatttgcaatttgcaatatccgagacaa |
| | | gggaggcctcatacagtcagatgttctatacctcctattccagctttcctttggaat |
| | | atccaatggattgatttactcctcagcattcatgattgtaggagacatagcttctgg |
| | | agaaaatgaacagaaagctgcttcaggttttactgctgtatttttgagtcttggtct |
| | | ggcatgtgggtcattaggaagctacctggttgtagcatttattctttagggccc |
| 22 | PpPMT1 | DPTVFNFNVQMLHYILGWVLHYLPSFLMARQLFLHHYLPSLYFGILALGHVFEIIHS |
| | FIG. 4 | YVFKNKQV |
| 23 | PpPMT2 | LFDKSDSFNVFLMGGLYPLLGWGLHFAPFVIMSRVTYVHHYLPALYFAMIVFCYLVS |
| | FIG. 4 | LLDKKLGHPALG |
| 24 | PpPMT4 | NSARSRLYNNLGFFFVGWCCHYLPFFLMSRQKFLHHYLPAHLIAAMFTAGFLEFIFT |
| | FIG. 4 | DNRTEEFK |
| 25 | PpPMT6 | SRQYWELVIKGFVPFFGWALHFAPFIVMQRVTYVHHYVPALYFAMFLLGFTVDYLTA |
| | FIG. 4 | KRNCYIKT |
| 26 | ScPMT1 | DSKVVNFHVQVIHYLLGFAVHYAPSFLMQRQMFLHHYLPAYYFGILALGHALDIIV |
| | FIG. 4 | SYVFRSKRQ |
| 27 | ScPMT2 | NPSNWNVFLMGGFYPLLAWGLHYMPFVIMSRVTYVHHYLPALYFALIILAYCFDAGL |
| | FIG. 4 | QKWSRSKCGR |
| 28 | ScPMT4 | KMTREKLYGPLMFFFVSWCCHYFPFFLMARQKFLHHYLPAHLIACLFSGALWEVIFS |
| | FIG. 4 | DCKSLDLE |
| 29 | ScPMT6 | DDQIWQITIQGIFPFISWMTHYLPFAMMGRVTYVHHYVPALYFAMLVFGFVLDFTLT |
| | FIG. 4 | RVHWMVKY |
| 30 | Pp PMT 5′ | TGCTCTCCGCGTGCAATAGAAACTAGTCGGCCCTGTACAATTAAAGCATACTCCCTG |
| | sequence | GTTAAAGTACCTCCTCCGAACTTGCTCTTGTTGATCAAAGTTTCTGACCTTGGGGCC |
| | | AGTCCCCAGCCACCAGGGCCAAACGCTTTATTGAGGATACGACGATACTTAATCTCT |
| | | GGAAGATAAAGTAGTCCATCTGGTGTGATTTCGACATCTTCGTTGCTAATTGGTTGA |
| | | CATAATATGTTACTACTTTCATTACTGAAGGAGCAAATACCTAGTCCATGGAACGAA |
| | | TCCGACCAATTGATTCCATCGCCACTTGTATTAGAGATTGGGGTGTCGTTTAACTGT |
| | | GAAGTTCCAAACAAAATTGATAAACTGCTCTCGTTCTTAGCTTGGCCACTTTTTGGA |
| | | GTCTCAATAGTAGCGTTTTGGCTCTCGTGAATTTTCTGCACAGAGTCGGATGAAGAA |
| | | GGTGCAAATGCTTCTAGCATTGTAGAGTCGACCACATAGAACCTTTTTAAAGAGTTA |
| | | TGAAAATAACTCTTGGTAGGGCCAAATACAACCCGATATCGTCTTAGCATAAGAGCT |
| | | GCTTCTTTGGAATATCGTTTCTTGTAAGTAATTACGTGTTGGCTAAACACTTAGAAG |
| | | TCAGTCGCGCATGCGGCCAAAAACAGACTAGGGATAGAAGATGAACTGACAAAAACA |
| | | TCAAGAAGGTGAAGACATTCATTCTATGAAAACTAGTTTTTATATAAAATTATGGTC |
| | | TGCATTTAGAGAGCAATGATGTAATCAAACATCAATAAGTGCTTGTCGCATCAATAT |
| | | TTAATAGGTAATCATGGAGTATTCTAGTCTACCGCCTTAAAAAAAGCTCACTCGATC |
| | | TAGTGCAGCTTGATTGTGTACTTCAATAGTATTCCAACGACCTTAACATCTTAACAC |
| | | CATGTAAATTTAAGATCCACGTATACGATACAATTTCTTTCAATATCAATTCTCGTT |
| | | CAAGCCAACTGATGATAAAATCAAGAAAGAGATCGAGAAAAGTTTCTTTGAACACTG |
| | | AAAAGGAGCTGAAAAATAGCCATATTTCTCTTGGAGATGAAAGATGGTACACT |
| 31 | Pp PMT4 | TAATTCTTCAAAGCCGAAAGAGCAATTGATTCTGTGGTTAAGTTTCTCGTCCTTTGT |
| | 3′ sequence | CGCTTTGCTACTAAGCATCATTGTTTGGACTTTCTTCTTTTTTGCTCCTCTAACATA |
| | | TGGTAATACTGCGCTTTCGGCGGAGGAGGTTCAGCAGCGACAATGGTTAGATATGAA |
| | | GCTCCAATTCGCCAAGTAAGAGTATACAATGTGTAGTTCAACGCAAAGGAAATTCTA |
| | | ACTTTCTGTGCAATCTGGTGACAATTTCTAAATAACTATCACAATTGGAAGAAGAGA |
| | | TTATCCCAAATCTTATCAAAAAATCGATGATTGCCAGTGCACAATTAGGCTTGAATT |
| | | TTTCTTGCAGCAACGAAGAGATTACTTCAGTGATGTTCATTAGCCTGAAATCTTCAC |
| | | TTTCGTGGTCTATCGGATTAGGAATTAGACCTTGTTTCATCGGCAGGTCGTATATGT |
| | | ATTCCACTTCTGGTTGAATAAAATCTTCGGGTGGTTTGTTTCTGAACATATATGAGA |
| | | TGGCTCCCACTGGACTGATATATTGCGAAACATAGTCCTCATTCAACCCTGCCTCCT |
| | | CGTAACATTCTTTCAGGCAAGTTTGCAAAGTGCCATTAGGATATTCCAAGCCTCCTG |
| | | CCACAGTATTATCTAACATACCGGGAAATGTTGGTTTGTGTCTGCTTCTCCTAGGTA |
| | | TCCAAAGTTGAATACTGTTAGGATCGGCAGAATTTTGCAAATATCCATTGATATGAA |
| | | CTCCATAAGTAACAACTCCCAAAATATTAGAAAAAGCCCTTTCCACCAACATGTACA |
| | | TCTTATGGTTATCGCAGTAAACTGCAAAAAGCTCATTTCTCCAACCGCTAAGGGTTT |
| | | CAAAGAGACGCTGATCTCTCCAACGCTGAGCTATCTTTGCAAACATCTGCGTTCTTT |
| | | TATTTTCGGTATCCAGACTAGGAATTATCTTGACTTCGTGTTTTTCATTATTTACTA |
| | | TCACAGCCTGTGTTTCGAACTCAAATTGTTTTGCCACCTTGGGAATTATATACCCTA |
| | | GT |
| 32 | NatR | GAGTTAGGTTCACATACGATTTAGGTGACACTATAGAACGCGGCCGCCAGCTGAAGC |
| | expression | TTCGTACGCTGCAGGTCGACGGATCCCCGGGTTAATTAAGGCGCGCCAGATCTGTTT |
| | cassette | AGCTTGCCTCGTCCCCGCCGGGTCACCCGGCCAGCGACATGGAGGCCCAGAATACCC |
| | NatR ORF | TCCTTGACAGTCTTGACGTGCGCAGCTCAGGGGCATGATGTGACTGTCGCCCGTACA |
| | 494-1066 | TTTAGCCCATACATCCCCATGTATAATCATTTGCATCCATACATTTTGATGGCCGCA |
| | Ashbya gossypii | CGGCGCGAAGCAAAAATTACGGCTCCTCGCTGCAGACCTGCGAGCAGGGAAACGCTC |
| | TEF1 promoter | CCCTCACAGACGCGTTGAATTGTCCCCACGCCGCGCCCCTGTAGAGAAATATAAAAG |
| | 106-493 | GTTAGGATTTGCCACTGAGGTTCTTCTTTCATATACTTCCTTTTAAAATCTTGCTAG |
| | Ashbya gossypii | GATACAGTTCTCACATCACATCCGAACATAAACAACCATGGGTACCACTCTTGACGA |
| | TEF1 | CACGGCTTACCGGTACCGCACCAGTGTCCCGGGGGACGCCGAGGCCATCGAGGCACT |
| | termination | GGATGGGTCCTTCACCACCGACACCGTCTTCCGCGTCACCGCCACCGGGGACGGCTT |
| | sequence | CACCCTGCGGGAGGTGCCGGTGGACCCGCCCCTGACCAAGGTGTTCCCCGACGACGA |
| | 1067-1313 | ATCGGACGACGAATCGGACGACGGGGAGGACGGCGACCCGGACTCCCGGACGTTCGT |
| | | CGCGTACGGGGACGACGGCGACCTGGCGGGCTTCGTGGTCATCTCGTACTCGGCGTG |
| | | GAACCGCCGGCTGACCGTCGAGGACATCGAGGTCGCCCCGGAGCACCGGGGGCACGG |
| | | GGTCGGGCGCGCGTTGATGGGGCTCGCGACGGAGTTCGCCGGCGAGCGGGGCGCCGG |
| | | GCACCTCTGGCTGGAGGTCACCAACGTCAACGCACCGGCGATCCACGCGTACCGGCG |
| | | GATGGGGTTCACCCTCTGCGGCCTGGACACCGCCCTGTACGACGGCACCGCCTCGGA |
| | | CGGCGAGCGGCAGGCGCTCTACATGAGCATGCCCTGCCCCTAATCAGTACTGACAAT |
| | | AAAAAGATTCTTGTTTTCAAGAACTTGTCATTTGTATAGTTTTTTTATATTGTAGTT |
| | | GTTCTATTTTAATCAAATGTTAGCGTGATTTATATTTTTTTTCGCCTCGACATCATC |
| | | TGCCCAGATGCGAAGTTAAGTGCGCAGAAAGTAATATCATGCGTCAATCGTATGTGA |
| | | ATGCTGGTCGCTATACTGCTGTCGATTCGATACTAACGCCGCCATCCAGTGTCGAAA |
| | | ACGAGCTCGAATTCATCGATGATATCAGATCCACTAGTGGCCTATGCGGCCGCGGAT |
| | | CTGCCGGTCTCCCTATAGTGAGTCGTATTCAC |
| 33 | Pp PMT2 | TTTGCTCCGTCTGTGATCATGT |
| | conserved | |
| | region with | |
| | point mutation | |
| | (YGLY17156) | |
| 34 | Pp PMT2 | TTTGCTCCGTTTGTGATCATGT |
| | conserved | |
| | region | |
| | without | |
| | point mutation | |
| | (YGLY17156) | |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A lower eukaryote host cell comprising a disruption in the expression of the endogenous protein mannosyltransferases 2 (PMT2) gene and a nucleic acid molecule encoding a mutant Pmt2p comprising at least one amino acid substitution, deletion, or insertion in the region of the Pmt2p protein comprising a conserved region having at least 80%, 90%, or 95% identity to the amino acid sequence of SEQ ID NO:9.

2. The lower eukaryote host cell of claim 1, wherein a serine residue replaces the phenylalanine residue at position 2 of SEQ ID NO:9.

3. The lower eukaryote host cell of claim 1, wherein the lower eukaryote is Pichia pastoris and the PMT2 gene encodes a Pmt2p protein having the amino acid sequence of SEQ ID NO:3 or the lower eukaryote is Saccharomyces cerevisiae and the PMT2 gene encodes a Pmt2p protein having the amino acid sequence of SEQ ID NO:7.

4. The lower eukaryote host cell of claim 1, wherein the lower eukaryote host cell is genetically engineered to produce glycoproteins comprising one or more mammalian- or human-like N-glycans.

5. The lower eukaryote host cell of claim 1, wherein the lower eukaryote host cell does not display Pmt4p activity.

6. The lower eukaryote host cell of claim 1, wherein the lower eukaryote host cell further includes a nucleic acid molecule encoding a therapeutic glycoprotein.

7. The lower eukaryote host cell of claim 6, wherein the therapeutic glycoprotein is erythropoietin (EPO); cytokines such as interferon α, interferon β, interferon γ, and interferon ω; and granulocyte-colony stimulating factor (GCSF); granulocyte macrophage-colony stimulating factor (GM-CSF); coagulation factors such as factor VIII, factor IX, and human protein C; antithrombin III; thrombin,; soluble IgE receptor α-chain; immunoglobulins such as IgG, IgG fragments, IgG fusions, and IgM; immunoadhesions and other Fc fusion proteins such as soluble TNF receptor-Fc fusion proteins; RAGE-Fc fusion proteins; interleukins; urokinase; chymase; urea trypsin inhibitor; IGF-binding protein; epidermal growth factor; growth hormone-releasing factor; annexin V fusion protein; angiostatin; vascular endothelial growth factor-2; myeloid progenitor inhibitory factor-1; osteoprotegerin; α-1-antitrypsin; α-feto proteins; DNase II; kringle 3 of human plasminogen; glucocerebrosidase; TNF binding protein 1; follicle stimulating hormone; cytotoxic T lymphocyte associated antigen 4-Ig; transmembrane activator and calcium modulator and cyclophilin ligand; glucagon like protein 1; or IL-2 receptor agonist.

8. The lower eukaryote host cell of claim 6, wherein the therapeutic glycoprotein is an anti-Her2 antibody, anti-RSV (respiratory syncytial virus) antibody, anti-TNFα antibody, anti-VEGF antibody, anti-CD3 receptor antibody, anti-CD41 7E3 antibody, anti-CD25 antibody, anti-CD52 antibody, anti-CD33 antibody, anti-IgE antibody, anti-CD11a antibody, anti-EGF receptor antibody, or anti-CD20 antibody.

9. A method for producing a recombinant heterologous protein in a lower eukaryote comprising:
expressing a nucleic acid molecule encoding the recombinant heterologous protein in a lower eukaryote host cell in which expression of the endogenous PMT2 gene is disrupted and which comprises a nucleic acid molecule encoding a mutant Pmt2p protein comprising an amino acid substitution, deletion, or insertion in a conserved region of the Pmt2p protein comprising an amino acid sequence with at least 80%, 90%, or 95% identity to the amino acid sequence comprising the SEQ ID NO:9 to produce the recombinant heterologous protein.

10. The method of claim 9, wherein the lower eukaryote is Pichia pastoris and the PMT2 gene encodes a Pmt2p protein having the amino acid sequence of SEQ ID NO:3 or the lower eukaryote is Saccharomyces cerevisiae and the PMT2 gene encodes a Pmt2p protein having the amino acid sequence of SEQ ID NO:7.

11. The method of claim 9, wherein the lower eukaryote host cell is genetically engineered to produce glycoproteins comprising one or more mammalian- or human-like N-glycans.

12. The method of claim 9, wherein the lower eukaryote host cell does not display Pmt4p activity.

13. A method for producing a recombinant heterologous protein in a lower eukaryote comprising:
(a) providing a lower eukaryote host cell in which expression of the endogenous PMT2 gene is disrupted and which comprises a nucleic acid molecule encoding a mutant Pmt2p protein comprising an amino acid substitution, deletion, or insertion in a conserved region of the Pmt2p protein comprising an amino acid sequence with at least 80%, 90%, or 95% identity to the amino acid sequence comprising the SEQ ID NO:9, and a second nucleic acid molecule encoding a recombinant heterologous protein; and
(b) growing the host cell in a medium comprising a Pmtp inhibitor for a time sufficient to produce the recombinant heterologous protein.

14. The method of claim 13, wherein the lower eukaryote is Pichia pastoris and the PMT2 gene encodes a Pmt2p protein having the amino acid sequence of SEQ ID NO:3 or the lower eukaryote is Saccharomyces cerevisiae and the PMT2 gene encodes a Pmt2p protein having the amino acid sequence of SEQ ID NO:7.

15. The method of claim 13, wherein the lower eukaryote host cell is genetically engineered to produce glycoproteins comprising one or more mammalian- or human-like N-glycans.

16. The method of claim 13, wherein the lower eukaryote host cell does not display Pmt4p activity.

17. The use of the host cells of claim 1-8 to produce a therapeutic protein for the treatment of a disease.

18. A process for producing recombinant therapeutic proteins comprising:
(a) providing a fungal host cell in which expression of the endogenous PMT2 gene is disrupted and which comprises a nucleic acid molecule encoding a Pmt2p protein comprising an amino acid substitution, deletion, or in a conserved region of the Pmt2p protein comprising an amino acid sequence with at least 80%, 90%, or 95% identity to the amino acid sequence comprising the SEQ ID NO:9, and second a nucleic acid molecule encoding a recombinant heterologous protein; and
(b) growing the host cell in a medium comprising a Pmtp inhibitor for a time sufficient to produce the recombinant heterologous protein.

19. The process of claim 18, wherein the fungal host cell is selected from the group consisting of Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Ogataea minuta, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, and Neurospora crassa

20. The process of claim 18, wherein the fungal host cell is Pichia pastoris or Saccharomyces cerevisiae.
